SEQUENCE LISTING (15 sequences)

SEQ ID NO: 1 - protein, 373 aa, Alligator mississippiensis
MATSASSHLSKAIKNMYMKLPQGEKVQAMYIWIDGTGEHLRCKTRTLDHEPKSIEDLPEWNFDGSSTFQSEGSNSDMYLQPAAMFRDPFRKDPNKLVLCEVFKYNRQTAETNLRHTCKRIMDMVSNQLPWFGMEQEYTLLGTDGHPFGWPSNGFPGPQGPYYCGVGADKAYGRDIVEAHYRACLYAGVKIGGTNAEVMPAQWEFQVGPCEGIEMGDHLWIARFILHRVCEDFGVIVSFDPKPIPGNWNGAGCHTNFSTKSMREEGGLKYIEEAIEKLGKRHQYHIRTYDPKGGLDNARRLTGFHETSSIHEFSAGVANRGASIRIPRSVGQVKKGYFEDRRPSANCDPYAVTEALVRTCLLNETGDEPFEYKN

SEQ ID NO: 2 - protein, 373 aa, Anolis carolinensis
MATSASSHLSKAIKHMYMKLPQGDKVQAMYIWIDGTGEFLRCKTRTLDHEPKNIEDLPEWNFDGSSTYQSEGSNSDMYLVPSAMFRDPFRKDPNKLVLCEVLKYNRKPAETNIRNSCERIMDMVSNQNPWFGMEQEYTLLGTDGHPFGWPSNGFPGPQGPYYCGVGADKAYGRDIVEAHYRACLYAGVNIGGTNAEVMPAQWEFQVGPCEGIEMGDHLWIARFILHRVCEDFGVIVSFDPKPIPGNWNGAGCHTNFSTKAMREEGGLKHIEEAIEKLGKRHQYHIRAYDPKGGLDNARRLTGFHETSNINEFSAGVANRGASIRIPRSVGQEKKGYFEDRRPSANCDPYAVTEALVRTCLLNETGDEPFEYKN

SEQ ID NO: 3 - protein, 376 aa, Lepisosteus oculatus
MIKMATSASSSLSKAVKQQYMELPQGDKVQAMYIWIDGTGEGLRCKTRTLDSEPKSIEDLPEWNFDGSSTYQSEGSNSDMYLIPAAMYRDPFRKDPNKLVLCEVLKYNRRPAETNLRSSCKRIMDMVANNRPWFGMEQEYTILGTDGHPFGWPSNGFPGPQGPYYCGVGADKAYGRDIVEAHYRACLYAGVQICGTNAEVMPAQWEFQVGPSEGIDMGDHLWIARFILHRVCEDFGVVASFDPKPIPGNWNGAGCHTNFSTKEMREENGLKYIEESIERLSRRHRYHIRAYDPKGGLDNARRLTGHNETSNIHEFSAGVANRGASIRIPRAVGQDKKGYFEDRRPSANCDPYSVTEALIRTCLLKEEGDEPVEYKN

SEQ ID NO: 4 - protein, 373 aa, Cricetulus griseus
MATSASSHLNKGIKQMYMSLPQGEKVQAMYIWVDGTGEGLRCKTRTLDCEPKCVEELPEWNFDGSSTFQSESSNSDMYLSPVAMFRDPFRKEPNKLVFCEVFKYNQKPAETNLRHTCKRIMDMVSNQHPWFGMEQEYTLLGTDGHPFGWPSDGFPGPQGLYYCGVGADKAYRRDIMEAHYRACLYAGVKITGTYAEVKHAQWEFQIGPCEGIRMGDHLWVARFILHRVCKDFGVIATFDSKPIPGNWNGAGCHTNFSTKTMREENGLKHIKEAIEKLSKRHRYHIRAYDPKGGLDNARRLTGFHKTSNINDFSAGVADRSASIRIPRTVGQEKKGYFEARCPSANCDPFAVTEAIVRTCLLNETGDQPFQYKN

SEQ ID NO: 5 - DNA, 1119 bp, Alligator mississippiensis
  source 1..1119: mol_type = other DNA
atggccacctcagcaagttcccacctgagcaaagctataaagaacatgtacatgaagctgccccagggcgagaaagtgcaagctatgtacatctggattgatgggactggagagcacttgcgctgtaaaacccggacattggaccatgaaccaaagagcattgaagatctaccagagtggaactttgatgggtctagtacctttcagtctgaaggctcaaacagcgatatgtatctacaacctgccgccatgttccgtgaccccttccgaaaggaccccaacaagctggttctctgtgaggtcttcaagtacaaccgccagacagcagagacaaatctaagacacacctgtaaacggattatggatatggtatccaaccagctcccctggtttggaatggagcaagaatacactctactagggacagatggacatccatttggctggccctccaatggattccctgggcctcagggcccatattactgtggagttggggcagacaaagcctacggcagagacattgtggaagcccattaccgagcatgtctctatgctggggttaaaattggagggacgaacgcagaggtgatgccagcacagtgggagttccaggtgggcccatgtgaagggattgagatgggagatcacctttggattgcacgcttcatcctccatcgggtgtgtgaagattttggggtgattgtgtcctttgatcccaagcccatccctgggaattggaatggagctggttgtcacactaacttcagcactaagtccatgagggaagaaggcggtctcaagtatattgaggaagccattgagaagctgggcaaacggcaccagtaccacatccgcacttatgacccgaaaggggggctggacaatgctaggcgccttacaggtttccatgaaacatccagcatccatgagttctcagctggtgtggccaaccgtggtgccagcattcgtatccccaggagcgtgggccaagtgaagaaaggctattttgaggaccgccgaccttctgccaattgtgacccttatgctgtgacagaggcactagtccgtacatgtctcctcaacgagactggggatgagccttttgagtacaagaac

SEQ ID NO: 6 - DNA, 1119 bp, Anolis carolinensis
  source 1..1119: mol_type = other DNA
atggccacctctgcaagttcccatttgagtaaagctataaagcatatgtacatgaagttgcctcagggggacaaggtgcaagctatgtacatctggatagatgggactggcgagtttctgcgttgtaagactcggacactggatcatgagcccaagaacattgaagatctaccagaatggaattttgatggctccagcacctaccagtccgagggctccaacagtgacatgtaccttgtgccttctgccatgttccgggaccctttccgcaaagaccccaacaagctggttctctgcgaggtcttaaagtacaaccgcaagcctgcagaaacaaatataaggaacagctgtgaaagaatcatggatatggtgtctaatcagaatccctggttcgggatggagcaagaatatactcttctgggaacagatggacatccctttggctggccttccaatggttttcctggaccccaaggcccatactactgtggagttggagcagacaaggcttatgggcgagacattgtggaagcccattatcgagcctgcctatatgctggtgtgaacattggtggcacaaatgcagaagttatgccagcgcagtgggagttccaggtgggtccatgcgaaggaattgagatgggtgaccacctctggattgctcggttcatccttcatagagtatgtgaagacttcggtgtcattgtgtcttttgaccccaagcctatcccaggcaactggaatggagctggatgccacacgaacttcagcactaaagccatgcgagaggaaggaggcctcaagcatattgaagaagctattgagaagctgggcaagcgccatcagtaccacatccgtgcctatgatcccaaaggggggctggataatgccaggcgtctgaccgggttccatgagacgtccaacatcaatgagttctctgctggagtagccaaccgtggtgccagcatccgcatcccaagaagcgtgggccaagagaagaaaggctactttgaagatcgccggccctccgccaactgtgacccttatgccgtgaccgaagcactagtccgcacttgtctcctcaacgaaaccggggatgagccctttgagtacaagaac

SEQ ID NO: 7 - DNA, 1128 bp, Lepisosteus oculatus
  source 1..1128: mol_type = other DNA
atgatcaagatggccacctccgccagctctagcctgagcaaggccgtcaagcagcagtacatggagctgccccagggcgacaaggtgcaggccatgtacatctggatcgacggcaccggggaggggctgcgctgcaagacccggactctggactccgagcccaagagcatcgaagacctgcccgagtggaacttcgacggctccagcacctaccagtccgagggctccaacagcgacatgtacctgatcccggcggccatgtaccgggaccccttccgcaaggaccccaacaagctggtcctgtgtgaagtgctgaagtacaacaggaggcctgcagagaccaacctccgcagctcctgcaagagaatcatggacatggtggccaacaatcgcccctggttcggcatggagcaggagtacaccatactgggcacggacggccacccatttggctggccctccaatggcttccccggaccccagggtccctattactgtggggtgggtgctgacaaggcttatgggcgagacattgtagaagctcattaccgagcctgcctgtacgccggagttcagatctgtggcaccaatgctgaagtcatgccagcccagtgggagttccaggttggccccagtgaaggaatcgacatgggagaccacttgtggattgccaggttcattctgcacagggtctgtgaggacttcggagtcgtggcctcatttgaccccaaacccatcccagggaactggaacggtgctggctgccacaccaacttcagcaccaaggagatgagggaagaaaacggcttgaagtacatcgaggagtcgattgagaggctgagcaggagacaccgttaccacatccgtgcctacgaccccaagggcggcctggacaacgccaggcgcctgacgggtcacaacgaaacctccaacatccacgagttctccgcgggcgtggccaaccgtggggccagcatccgcatccctcgcgccgtgggccaggacaagaagggctacttcgaggaccggcgcccctccgccaactgcgacccctacagcgtgacggaggcgctgatccgcacatgtttactgaaggaagagggagatgaacctgtggagtacaagaac

SEQ ID NO: 8 - DNA, 1122 bp, Cricetulus griseus
  source 1..1122: mol_type = other DNA
atggccacctcagcaagttcccacttgaacaaaggcatcaagcaaatgtacatgtccctgccccagggtgagaaagtccaagccatgtatatctgggttgatggtaccggagaaggactgcgctgcaaaacccgcaccctggactgtgagcccaagtgtgtagaagagttacctgagtggaattttgatggctctagtacctttcagtctgagagctccaacagtgacatgtatctcagccctgttgccatgtttcgggaccccttccgcaaagagcccaacaagctggtgttctgtgaagtcttcaagtacaaccagaagcctgcagagaccaatttaagacacacgtgtaaacggataatggacatggtgagcaaccagcacccctggtttggaatggaacaggagtatactctcttgggaacagatgggcacccttttggttggccttccgatggcttccctgggccccaaggtctgtattactgtggtgtgggcgcagacaaagcctatcgcagggacatcatggaggctcactaccgtgcctgcttgtatgctggggtcaagattacaggaacatatgctgaggtcaagcatgcccagtgggagttccaaataggaccctgtgaaggaatccgcatgggagatcatctctgggtggcccgtttcatcttgcatcgagtatgtaaagactttggagtaatagcaacctttgactccaagcccattcctgggaactggaatggtgcaggctgccataccaactttagtaccaagaccatgcgggaggagaatggtctgaagcacatcaaggaggccattgagaaactaagcaagcggcaccggtaccatattcgagcctacgatcccaagggggggctggacaatgcccgtcgtctgactgggttccacaaaacgtccaacatcaacgacttttctgctggcgtcgccgaccgcagtgccagcatccgcattccccggactgtcggccaggagaagaaaggttactttgaagcccgctgcccctctgccaattgtgacccctttgcagtgacagaagccatcgtccgcacatgccttctcaatgagactggcgaccagcccttccaatacaaaaactaa

SEQ ID NO: 9 - protein, 22 aa, synthetic construct
  REGION 1..22: note = Linker
GSGATNFSLLKQAGDVEENPGP

SEQ ID NO: 10 - protein, 23 aa, synthetic construct
  REGION 1..23: note = Linker
GSGQCTNYALLKLAGDVESNPGP

SEQ ID NO: 11 - protein, 25 aa, synthetic construct
  REGION 1..25: note = Linker
GSGVKQTLNFDLLKLAGDVESNPGP

SEQ ID NO: 12 - protein, 21 aa, synthetic construct
  REGION 1..21: note = Linker
GSGEGRGSLLTCGDVEENPGP

SEQ ID NO: 13 - protein, 40 aa, synthetic construct
  REGION 1..40: note = PEST degron
SHGFPPEVEEQAAGTLPMSCAQESGMDRHPAACASARINV

SEQ ID NO: 14 - protein, 123 aa, synthetic construct
  REGION 1..123: note = ODD degron
EFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETT

SEQ ID NO: 15 - protein, 50 aa, synthetic construct
  REGION 1..50: note = IkBa degron
IQQQLGQLTLENLQMLPESEDEESYDTESEFTEFTEDELPYDDCVFGGQR

(19) United States
(12) Patent Application Publication
(10) Pub. No.: US 2025/0066491 A1
(43) Pub. Date: Feb. 27, 2025

(54) NOVEL MARKERS FOR RECOMBINANT PRODUCTION SYSTEM

(71) Applicant: Shanghai ZhenGe Biotechnology Co., Ltd., Shanghai (CN)

(72) Inventors: Wei Shao, Perry Hall, MD (US); Jinggong Chen, Shanghai (CN); Ying Xu, Shanghai (CN); Yu Long, Ellicott City, MD (US); Jing Zhang, Shanghai (CN); Xingxing Liu, Shanghai (CN); Honghui Yuan, Shanghai (CN); Yali Wei, Shanghai (CN); Jinrong Liu, Shanghai (CN); Wei Wu, Shanghai (CN); Weidong Hao, Potomac, MD (US); Jianxin Chen, Shanghai (CN)

(21) Appl. No.: 18/724,185

(22) PCT Filed: Dec. 26, 2022
(86) PCT No.: PCT/CN2022/141848

§ 371 Date: Jun. 25, 2024

(30) Foreign Application Priority Data
Dec. 27, 2021 (WO) PCT/CN2021/141584

(51) Int. Cl. C07K 16/28; C12N 9/00; C12N 15/10

(52) U.S. Cl. CPC C07K 16/2875; C12N 9/93; C12N 15/1082; C12Y 603/01002

(57) ABSTRACT

Provided are novel selectable markers and uses thereof. Specifically, provided are uses of nucleotide sequences encoding a glutamine synthetase (GS) derived from Alligator, green anole, or spotted gar as selectable markers for identifying genomic loci with high transcriptional activity or host cells having high productivity of a protein of interest, and/or for accelerating the identification process. Related methods of screening, methods of production, and expression systems are also included.

This application claims priority to PCT Patent Application No. PCT/CN2021/141584, filed Dec. 27, 2021, which is incorporated herein by reference in its entirety.

### 1. FIELD

The present invention relates to molecular biology and cell biology. Provided herein include selectable markers for recombinant protein production and related methods of uses.

### 2. BACKGROUND

Selectable markers are commonly used in recombinant protein production. When generating clones expressing a recombinant protein, host cells are usually transfected with a DNA vector encoding both the protein of interest and the selectable marker. The selectable marker allows the selection of cell clones having the expression vector as well as high-producer clones. Glutamine synthetase (GS) has been widely used as a selectable marker in recombinant protein production in eukaryotic cells, such as the Chinese hamster ovary (CHO) cells. Nearly all selectable marker currently used in GS system is Cricetulus griseus-derived GS, which has relatively low selection efficiency and yield. More effective and efficient manufacturing processes are essential to support the development of innovative biologics and biosimilars, which require highly productive cell lines with desired quality attributes. Thus, there is an unmet and urgent need for expression systems with improved effectiveness and efficiency in cell line generation processes, especially the selection step for top-producing clonal cell lines. The vectors, cells, expression systems provided herein address this need and provide related advantages.

### 3. SUMMARY

Provided herein are uses of a nucleotide sequence encoding a glutamine synthetase (GS) as a selectable marker, wherein the GS is an Alligator GS, a green anole GS, or a spotted gar GS.

In some embodiments of the uses provided herein, the GS is an Alligator GS derived from Alligatoridae. In some embodiments, the GS has an amino acid sequence that is at least 90%, at least 92%, at least 95%, at least 98%, at least 99%, or 100% identical to SEQ ID NO:1. In some embodiments, the GS has reduced activity compared to a wild-type Alligator GS. In some embodiments, the GS has the amino acid sequence of SEQ ID NO:1.

In some embodiments of the uses provided herein, the GS is a green anole GS derived from Dactyloidae. In some embodiments, the GS has an amino acid sequence that is at least 90%, at least 92%, at least 95%, at least 98%, at least 99%, or 100% identical to SEQ ID NO:2. In some embodiments, the GS has reduced activity compared to a wild-type green anole GS. In some embodiments, the GS has the amino acid sequence of SEQ ID NO:2.

In some embodiments of the uses provided herein, the GS is a spotted gar GS derived from Lepisosteidae. In some embodiments, the GS has an amino acid sequence that is at least 90%, at least 92%, at least 95%, at least 98%, at least 99%, or 100% identical to SEQ ID NO:3. In some embodiments, the GS has reduced activity compared to a wild-type spotted gar GS. In some embodiments, the GS has the amino acid sequence of SEQ ID NO:3.

In some embodiments of the uses provided herein, the amino acid sequence of the GS provided herein comprises SEQ ID NO:4 with an amino acid substitution at a position selected from the group consisting of: H8, N10, G12, I13, Q15, M16, S19, E24, V33, G39, C49, C53, V54, E56, F68, S72, S80, V82, F85, E92, F98, F102, Q106, K107, P108, L113, H115, T116, K118, S125, Q127, H128, L139, D152, L160, R172, M176, K189, T191, Y194, K198, H199, I206, C209, R213, V220, K230, I235, A236, T237, S240, T260, N265, H269, K271, A273, K276, S278, K279, R282, A287, F303, H304, K305, N308, N310, D311, D318, S320, T328, E332, A339, C341, F349, A350, I355, V356, N362, T364, Q367, F369, and Q370.

In some embodiments of the uses provided herein, the amino acid sequence of the GS provided herein comprises SEQ ID NO:4 with an amino acid substitution at a position selected from the group consisting of: N10, G12, S19, V33, C49, C53, V54, E56, S72, S80, V82, E92, F98, Q106, H128, D152, L160, R172, M176, T191, Y194, K198, H199, I206, R213, V220, K230, T237, S240, T260, K271, K305, D311, D318, S320, T328, A339, C341, F349, I355, Q367, and Q370. In some embodiments, the amino acid sequence of the GS comprises SEQ ID NO:4 with about 3, about 5, about 10, about 15, about 20, about 25, about 30, about 35, or about 40 amino acid substitutions at positions selected from the group consisting of: N10, G12, S19, V33, C49, C53, V54, E56, S72, S80, V82, E92, F98, Q106, H128, D152, L160, R172, M176, T191, Y194, K198, H199, I206, R213, V220, K230, T237, S240, T260, K271, K305, D311, D318, S320, T328, A339, C341, F349, I355, Q367, and Q370.

In some embodiments, provided herein are uses of a nucleotide sequence encoding a GS as a selectable marker, wherein the GS comprises a catalytic domain from an Alligator GS, a green anole GS, or a spotted gar GS. In some embodiments, the GS comprises a catalytic domain from an Alligator GS having an amino acid sequence that is at least 90%, at least 92%, at least 95%, at least 98%, at least 99%, or 100% identical to SEQ ID NO:1. In some embodiments, the catalytic domain has an amino acid sequence that is at least 90%, at least 92%, at least 95%, at least 98%, at least 99%, or 100% identical to amino acids 110-359 of SEQ ID NO:1. In some embodiments, the GS comprises a catalytic domain from a green anole GS having an amino acid sequence that is at least 90%, at least 92%, at least 95%, at least 98%, at least 99%, or 100% identical to SEQ ID NO:2. In some embodiments, the catalytic domain has an amino acid sequence that is at least 90%, at least 92%, at least 95%, at least 98%, at least 99%, or 100% identical to amino acids 110-359 of SEQ ID NO:2. In some embodiments, the GS comprises a catalytic domain from a spotted gar GS having an amino acid sequence that is at least 90%, at least 92%, at least 95%, at least 98%, at least 99%, or 100% identical to SEQ ID NO:3. In some embodiments, the catalytic domain has an amino acid sequence that is at least 90%, at least 92%, at least 95%, at least 98%, at least 99%, or 100% identical to amino acids 113-362 of SEQ ID NO:3.

In some embodiments of the uses provided herein, the nucleotide sequence encoding the GS is operatively linked to a mRNA destabilizing element. In some embodiments, the GS comprises a degron. In some embodiments, the degron has an amino acid sequence selected from the group consisting of SEQ ID NOs:13-15.

In some embodiments, the uses provided herein are for identifying a genomic locus with high transcriptional activity. In some embodiments, the uses provided herein are for identifying a host cell capable of producing a protein of interest (POI). In some embodiments, the uses provided herein are for recombinant production of a POI. In some embodiments, the uses provided herein are for recombinant protein production in a mammalian cell. In some embodiments, the mammalian cell is a Chinese Hamster Ovary (CHO) cell. In some embodiments, the POI is selected from the group consisting of an antibody, an enzyme, a soluble protein, a secreted protein, a membrane protein, and a fusion protein.

Provided herein are also deoxyribonucleic acid (DNA) vectors suitable for recombinant protein production or genomic integration, comprising a nucleotide sequence encoding a GS (a GS-encoding sequence), wherein the GS is an Alligator GS, a green anole GS, or a spotted gar GS.

In some embodiments of the vectors provided herein, the encoded GS is an Alligator GS derived from Alligatoridae. In some embodiments, the GS has an amino acid sequence that is at least 90%, at least 92%, at least 95%, at least 98%, at least 99%, or 100% identical to SEQ ID NO:1. In some embodiments, the GS has reduced activity compared to a wild-type Alligator GS. In some embodiments, the GS has the amino acid sequence of SEQ ID NO:1. In some embodiments, the GS-encoding sequence is at least 80% identical to SEQ ID NO:5.

In some embodiments of the vectors provided herein, the encoded GS is a green anole GS derived from Dactyloidae. In some embodiments, the GS has an amino acid sequence that is at least 90%, at least 92%, at least 95%, at least 98%, at least 99%, or 100% identical to SEQ ID NO:2. In some embodiments, the GS has reduced activity compared to a wild-type green anole GS. In some embodiments, the GS has the amino acid sequence of SEQ ID NO:2. In some embodiments, the GS-encoding sequence is at least 80% identical to SEQ ID NO:6.

In some embodiments of the vectors provided herein, the encoded GS is a spotted gar GS derived from Lepisosteidae. In some embodiments, the GS has an amino acid sequence that is at least 90%, at least 92%, at least 95%, at least 98%, at least 99%, or 100% identical to SEQ ID NO:3. In some embodiments, the GS has reduced activity compared to a wild-type spotted gar GS. In some embodiments, the GS has the amino acid sequence of SEQ ID NO:3. In some embodiments, the GS-encoding sequence is at least 80% identical to SEQ ID NO:7.

In some embodiments of the vectors provided herein, the amino acid sequence of the encoded GS comprises SEQ ID NO:4 with an amino acid substitution at a position selected from the group consisting of: H8, N10, G12, I13, Q15, M16, S19, E24, V33, G39, C49, C53, V54, E56, F68, S72, S80, V82, F85, E92, F98, F102, Q106, K107, P108, L113, H115, T116, K118, S125, Q127, H128, L139, D152, L160, R172, M176, K189, T191, Y194, K198, H199, I206, C209, R213, V220, K230, I235, A236, T237, S240, T260, N265, H269, K271, A273, K276, S278, K279, R282, A287, F303, H304, K305, N308, N310, D311, D318, S320, T328, E332, A339, C341, F349, A350, I355, V356, N362, T364, Q367, F369, and Q370.

In some embodiments, the amino acid sequence of the GS comprises SEQ ID NO:4 with an amino acid substitution at a position selected from the group consisting of: N10, G12, S19, V33, C49, C53, V54, E56, S72, S80, V82, E92, F98, Q106, H128, D152, L160, R172, M176, T191, Y194, K198, H199, I206, R213, V220, K230, T237, S240, T260, K271, K305, D311, D318, S320, T328, A339, C341, F349, I355, Q367, and Q370. In some embodiments, the amino acid sequence of the GS comprises SEQ ID NO:4 with about 3, about 5, about 10, about 15, about 20, about 25, about 30, about 35, or about 40 amino acid substitutions at positions selected from the group consisting of: N10, G12, S19, V33, C49, C53, V54, E56, S72, S80, V82, E92, F98, Q106, H128, D152, L160, R172, M176, T191, Y194, K198, H199, I206, R213, V220, K230, T237, S240, T260, K271, K305, D311, D318, S320, T328, A339, C341, F349, I355, Q367, and Q370.

Provided herein are also DNA vectors suitable for recombinant protein production or genomic integration, comprising a nucleotide sequence encoding a GS (a GS-encoding sequence), wherein the GS comprises a catalytic domain from an Alligator GS, a green anole GS, or a spotted gar GS. In some embodiments, the GS comprises a catalytic domain from an Alligator GS having an amino acid sequence that is at least 90%, at least 92%, at least 95%, at least 98%, at least 99%, or 100% identical to SEQ ID NO:1. In some embodiments, the catalytic domain has an amino acid sequence that is at least 90%, at least 92%, at least 95%, at least 98%, at least 99%, or 100% identical to amino acids 110-359 of SEQ ID NO:1. In some embodiments, the GS comprises a catalytic domain from a green anole GS having an amino acid sequence that is at least 90%, at least 92%, at least 95%, at least 98%, at least 99%, or 100% identical to SEQ ID NO:2. In some embodiments, the catalytic domain has an amino acid sequence that is at least 90%, at least 92%, at least 95%, at least 98%, at least 99%, or 100% identical to amino acids 110-359 of SEQ ID NO:2. In some embodiments, the GS comprises a catalytic domain from a spotted gar GS having an amino acid sequence that is at least 90%, at least 92%, at least 95%, at least 98%, at least 99%, or 100% identical to SEQ ID NO:3. In some embodiments, the catalytic domain has an amino acid sequence that is at least 90%, at least 92%, at least 95%, at least 98%, at least 99%, or 100% identical to amino acids 113-362 of SEQ ID NO:3.

In some embodiments of the vectors provided herein, the GS-encoding sequence is operatively linked to an mRNA-destabilizing element. In some embodiments of the vectors provided herein, the encoded GS comprises a degron. In some embodiments, the degron has an amino acid sequence selected from the group consisting of SEQ ID NOs:13-15.

In some embodiments of the vectors provided herein, the GS-encoding sequence is operatively linked to Simian vacuolating virus 40 (SV40) promoter. In some embodiments of the vectors provided herein, the GS-encoding sequence is operatively linked to a poly(A) tail.

In some embodiments, the vector provided herein is suitable for recombinant protein production and further comprises an expression cassette. In some embodiments, the vectors provided herein comprise two or more expression cassettes. In some embodiments, the expression cassette comprises a nucleotide sequence encoding a POI (POI-encoding sequence). In some embodiments, the POI is an antibody, an enzyme, a soluble protein, a secreted protein, a membrane protein, or a fusion protein. In some embodiments, the POI is an antibody selected from the group consisting of an IgG1 antibody, an IgG2 antibody, an IgG3 antibody, an IgG4 antibody, an IgA antibody, an IgM antibody, a Fab, a Fab′, a F(ab′)2, a Fv, a scFv, a (scFv)2, a single domain antibody (sdAb), a single chain antibody (scAb), and a heavy chain antibody (HCAb). In some embodiments, the POI is an antibody selected from the group consisting of a monoclonal antibody, a bispecific antibody, a multi-specific antibody, a bivalent antibody, and a multivalent antibody. In some embodiments, the POI consists of one or more copies of the same polypeptide. In some embodiments, the POI comprises two different polypeptides. In some embodiments, the POI is an antibody comprising a light chain and a heavy chain, each encoded by a separate nucleotide sequence on the vector.

In some embodiments, the vectors provided herein are suitable for genomic integration.

In some embodiments, provided herein are uses of the vectors provided herein for identifying host cells capable of producing the POI.

In some embodiments, provided herein are uses of the vectors provided herein for identifying a genomic locus with high transcriptional activity.

In some embodiments, provided herein are methods for identifying a host cell capable of producing a POI, comprising introducing the vector described herein into a population of host cells, culturing the population of host cells in a glutamine-free medium, wherein the host cell capable of growing in the culture medium is identified as the host cell capable of producing the POI.

In some embodiments, provided herein are methods for identifying a genomic locus with high transcriptional activity, comprising introducing the vector described herein into a population of host cells, culturing the population of host cells in a glutamine-free medium, wherein the host cell capable of growing in the culture medium is identified as the host cell having the GS-encoding sequence inserted at a genomic locus with high transcriptional activity. In some embodiments, methods provided herein further comprise sequencing the genome of the identified host cell to locate the genomic locus with high transcriptional activity.

In some embodiments of the methods provided herein, the population of host cells are cultured in the presence of a GS inhibitor. In some embodiments, the GS inhibitor is methionine sulfoximine (MSX).

In some embodiments, provided herein are host cells comprising the vector disclosed herein. In some embodiments, the host cell has a wild-type endogenous GS. In some embodiments, the endogenous GS of the host cell has reduced activity or is knocked out. In some embodiments, the host cell is a mammalian cell. In some embodiments, the host cell is a CHO cell.

In some embodiments, provided herein are uses of the host cells disclosed herein for in vitro production of the POI. In some embodiments, provided herein are in vitro methods of producing a POI comprising culturing the host cells disclosed herein under conditions and for sufficient time to produce the POI.

In some embodiments, provided herein are in vitro methods of producing a POI comprising replacing the GS-encoding sequence with a POI-encoding sequence in the host cell identified in the method described herein, and culturing the host cell under conditions and for sufficient time to produce the POI. In some embodiments, the methods further comprise separating the POI from other components in the culture. In some embodiments, the separating comprises extraction, continuous liquid-liquid extraction, pervaporation, membrane filtration, membrane separation, reverse osmosis, electrodialysis, distillation, crystallization, centrifugation, extractive filtration, ion exchange chromatography, absorption chromatography, or ultrafiltration.

In some embodiments, provided herein are expression systems for in vitro production of a POI comprising the DNA vector disclosed herein and a host cell. In some embodiments, the host cell is a CHO cell. In some embodiments, the expression systems provided herein further comprise a glutamine-free culture medium. In some embodiments, the expression systems provided herein further comprise a GS inhibitor. In some embodiments, the expression systems provided herein further comprise a means for introducing the vector into the host cell. In some embodiments, the expression system provided herein further comprise is contained in a kit.

### 4. BRIEF DESCRIPTION OF DRAWINGS

FIG. 1 provides the map of plasmids containing GS from different species as the selectable marker. The Origin of Replication and AmpR gene allowed the amplification and preparation of plasmids from bacteria. The expression of GS was driven by SV40 promoter. Sequences encoding UnaG (the green fluorescent protein from the eel) and 2A self-cleaving peptide were cloned to the upstream of GS, which allowed the equal molar expression of UnaG and GS. Sequences encoding the heavy chain and light chain of denosumab were cloned into two separated expression cassettes, in which the expression was driven by CMV promoter.

FIG. 2 provides results of the flow cytometry analysis of CHO cells cultured in the absence or presence of 50 μM methionine sulfoximine (MSX) at Day 21 post-electroporation. Cells were electroporated with denosumab expression vector containing GS selectable marker from indicated species.

FIG. 3 provides dot plots of denosumab expression. Cells were electroporated with denosumab expression vector containing GS selectable marker from indicated species, then divided into 300 pools in 96-well plates. All pools were cultured in the presence of 50 μM MSX for 21 days. Denosumab levels in the supernatant from each pool were measured by Octet Qke Label-free system. Denosumab expression from all positive pools (left) or top 30 pools (right) were plotted. Horizontal lines indicate median values.

FIG. 4 provides titer distribution of ELISA-based analysis of denosumab expression. Cells were electroporated with denosumab expression vector containing GS selectable marker from indicated species, then divided into 300 pools in 96-well plates. All pools were cultured in the presence of 50 μM MSX for 21 days. Denosumab levels in the supernatant from each pool were measured by Octet Qke Label-free System.

FIG. 5 provides amino acid sequence alignment of the GS from Chinese hamster (Cricetulus griseus), Alligator (Alligator mississippiensis), green anole (Anolis carolinensis), and spotted gar (Lepisosteus oculatus).

### 5. DETAILED DESCRIPTION

Provided herein are expression systems using a GS derived from Alligator, green anole, or spotted gar, as a selectable marker in, for example, recombinant protein production, and methods of screening for cell clones with high productivity. Without being bound by theory, the inventions provided herein are based on the surprising discovery that GS from species that are phylogenetically remote from the CHO host cell, in particular, the GS derived from Alligator, green anole, and spotted gar, can serve as highly effective selectable markers, and the uses thereof result in significant increases in not only the ratio of positive cell clones expressing the protein of interest (“POI”), but also the expression level of the POI in the host cells (e.g., CHO cells). As disclosed herein, when GS derived from Alligator, green anole, or spotted gar is used as the selectable marker, efficient identification of cell clones having high productivity of the recombinant POI was achieved. Accordingly, in some embodiments, provided herein are uses of a nucleotide sequence encoding a GS (a GS-encoding sequence) as a selectable marker, wherein the GS is an Alligator GS, a green anole GS, or a spotted gar GS; provided herein are also expression vectors comprising a GS-encoding sequence, wherein the GS is an Alligator GS, a green anole GS, or a spotted gar GS, and methods of uses thereof, expression systems and kits comprising such vectors are also provided herein.

Before the present disclosure is further described, it is to be understood that the disclosure is not limited to the particular embodiments set forth herein, and it is also to be understood that the terminology used herein is for the purpose of describing particular embodiments, and is not intended to be limiting.

Unless otherwise defined herein, scientific and technical terms used in the present disclosures shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

The term “a” or “an” entity refers to one or more of that entity; for example, “a vector,” is understood to represent one or more vectors.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “identical,” percent “identity,” and their grammatical equivalents as used herein in the context of two or more polynucleotides or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two polynucleotides or polypeptides provided herein are substantially identical, meaning they have at least 70%, at least 75%, at least 80%, at least 85%, at least 90%, and in some embodiments at least 95%, at least 96%, at least 97%, at least 98%, or at least 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a POI. Unless otherwise specified, the percentage of identity as used herein is calculated using a global alignment (i.e., the two sequences are compared over their entire length).

The term “about” as used herein refers to the usual error range for the respective value readily known to the skilled person in this technical field. Reference to “about” a value or parameter herein includes and describes embodiments that are directed to that value or parameter per se. For example, description referring to “about X” includes description of “X.” In some embodiments, “about” indicates a value of up to ±10% of a recited value, e.g., ±1%, 2%, 3%, ±4%, 5%, ±6%, ±7%, ±8%, ±9%, or ±10%. In various embodiments, the term “about” encompasses variations of ±5%, ±2%, ±1%, or ±0.5% of the numerical value of the number. In certain embodiments, the term “about” encompasses variations of ±5% of the numerical value of the number. In certain embodiments, the term “about” encompasses variations of ±2% of the numerical value of the number. In certain embodiments, the term “about” encompasses variations of ±1% of the numerical value of the number.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Exemplary genes and polypeptides are described herein with reference to GenBank numbers, GI numbers, Uniprot numbers, and/or SEQ ID NOS. It is understood that one skilled in the art can readily identify homologous sequences by reference to sequence sources, including but not limited to Uniprot (uniprot.org/), GenBank (ncbi.nlm.nih.gov/genbank/) and/or EMBL (embl.org/).

### 5.1 Selectable Markers

To produce recombinant proteins at industrial scale, identification of cell clones that can produce high amounts of recombinant proteins is crucial. Selectable markers provided herein allow efficient identification of cell clones having high productivity of a POI. The high productivity of the cell is achieved by having the expression vector integrated in a highly transcriptionally active site or having multiple copies of expression vector present in the cell. As such, provided herein are uses of a nucleotide sequence encoding a glutamine synthetase (GS) as a selectable marker, wherein the GS is an Alligator GS, a green anole GS, or a spotted gar GS. In some embodiments, provided herein are uses for recombinant protein production. In some embodiments, provided herein are uses for identifying cell clones capable of producing a POI. In some embodiments, provided herein are uses for identifying genomic loci for integration of transgenes for efficient expression.

As used herein and commonly understood in the art, a “selectable marker” is a gene that confers a trait to its carrier that allows artificial selection. The trait is usually a positive trait, such as resistance to an antibiotic, or a key enzymatic activity. A selectable marker is usually an integral part of a vector, such as an expression vector, and is commonly used in molecular biology and genetic engineering to indicate the success of a procedure to introduce the vector into a cell. Once a vector containing a selectable marker is introduced into a population of host cells, the cells can be cultured in a medium in which their survival and growth require the expression of the selectable marker. As such, a selectable marker enables selection of the cells that have successfully taken up and expressed the vector. The selection condition can be adjusted for different levels of stringency. Generally, the more stringent the culture condition is, the higher expression of the selectable marker that is required from the host cells to grow at such condition.

Glutamine synthetase, or GS, is an enzyme classified under Enzyme Commission (EC) number 6.3.1.2. GS catalyzes the ATP-dependent conversion of glutamate and ammonia to glutamine and plays key roles in nitrogen metabolism. The biochemical reaction catalyzed by GS can be represented as: ATP+L-glutamate+NH3<=>ADP+phosphate+L-glutamine. The enzymatic activity that catalyzes the above reaction is herein referred to as “GS activity.”

A wild-type GS typically have two major domains, the beta grasp domain (e.g., from amino acid residue at position 30 to amino acid residue at position 104 in a wild-type hamster GS, SEQ ID NO:4) and the catalytic domain (e.g., from amino acid residue at position 134 to amino acid residue at position 351 in a wild-type hamster GS, SEQ ID NO:4).

GS is a popular selectable marker in mammalian cell lines. In some cell lines, the endogenous GS can be inactivated or inhibited. As such, these cells cannot synthesize glutamine on their own and can grow only if glutamine is added to the culture medium, or in a glutamine-free culture medium, if they have incorporated expression vectors comprising GS gene. Positive selection can be maintained by using a glutamine-free culture medium. Also, inhibitors of GS activity, including methionine sulfoximine (MSX) and derivatives thereof, phosphorus containing analogues of glutamic acid, and bisphosphonates, at different concentrations can be used for creating different levels of selection stringency. Increase of a GS inhibitor can select for cells with higher expression of GS because GS activity is inhibited and only the cells with higher level of GS can grow under such treatment. Thus, cells having amplified copy number of the expression cassette in the chromosome, or those having the expression cassette inserted at highly transcriptionally active site can be identified.

In some embodiments, provided herein are uses of a nucleotide sequence encoding a GS (a GS-encoding sequence) as a selectable marker, wherein the GS is an Alligator GS, a green anole GS, or a spotted gar GS. As used herein and understood in the art, the term “encode” or its grammatical equivalents refer to the inherent property of specific nucleotide sequences to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein. Unless otherwise specified, a “nucleotide sequence encoding” an amino acid sequence can be any nucleotide sequences that are degenerate versions of each other and that encode the same amino acid sequence. Nucleotide sequences that encode proteins and RNA can include introns.

TABLE 1
Exemplary GS amino acid sequences and nucleotide sequences.

| GS | Amino Acid Sequences | Nucleotide Sequences |
|---|---|---|
| Alligator | MATSASSHLSKAIKNMYMKLPQGE | ATGGCCACCTCAGCAAGTTCCCACCTGAGCA |
| (Alligator | KVQAMYIWIDGTGEHLRCKTRTLD | AAGCTATAAAGAACATGTACATGAAGCTGCC |
| mississippiensis) | HEPKSIEDLPEWNFDGSSTFQSEGSN | CCAGGGCGAGAAAGTGCAAGCTATGTACATC |
| | SDMYLQPAAMFRDPFRKDPNKLVL | TGGATTGATGGGACTGGAGAGCACTTGCGCT |
| | CEVFKYNRQTAETNLRHTCKRIMD | GTAAAACCCGGACATTGGACCATGAACCAAA |
| | MVSNQLPWFGMEQEYTLLGTDGHP | GAGCATTGAAGATCTACCAGAGTGGAACTTT |
| | FGWPSNGFPGPQGPYYCGVGADKA | GATGGGTCTAGTACCTTTCAGTCTGAAGGCT |
| | YGRDIVEAHYRACLYAGVKIGGTN | CAAACAGCGATATGTATCTACAACCTGCCGC |
| | AEVMPAQWEFQVGPCEGIEMGDHL | CATGTTCCGTGACCCCTTCCGAAAGGACCCC |
| | WIARFILHRVCEDFGVIVSFDPKPIPG | AACAAGCTGGTTCTCTGTGAGGTCTTCAAGT |
| | NWNGAGCHTNFSTKSMREEGGLKY | ACAACCGCCAGACAGCAGAGACAAATCTAA |
| | IEEAIEKLGKRHQYHIRTYDPKGGL | GACACACCTGTAAACGGATTATGGATATGGT |
| | DNARRLTGFHETSSIHEFSAGVANR | ATCCAACCAGCTCCCCTGGTTTGGAATGGAG |
| | GASIRIPRSVGQVKKGYFEDRRPSA | CAAGAATACACTCTACTAGGGACAGATGGAC |
| | NCDPYAVTEALVRTCLLNETGDEPF | ATCCATTTGGCTGGCCCTCCAATGGATTCCCT |
| | EYKN | GGGCCTCAGGGCCCATATTACTGTGGAGTTG |
| | (SEQ ID NO: 1) | GGGCAGACAAAGCCTACGGCAGAGACATTGT |
| | | GGAAGCCCATTACCGAGCATGTCTCTATGCT |
| | | GGGGTTAAAATTGGAGGGACGAACGCAGAG |
| | | GTGATGCCAGCACAGTGGGAGTTCCAGGTGG |
| | | GCCCATGTGAAGGGATTGAGATGGGAGATCA |
| | | CCTTTGGATTGCACGCTTCATCCTCCATCGGG |
| | | TGTGTGAAGATTTTGGGGTGATTGTGTCCTTT |
| | | GATCCCAAGCCCATCCCTGGGAATTGGAATG |
| | | GAGCTGGTTGTCACACTAACTTCAGCACTAA |
| | | GTCCATGAGGGAAGAAGGCGGTCTCAAGTAT |
| | | ATTGAGGAAGCCATTGAGAAGCTGGGCAAAC |
| | | GGCACCAGTACCACATCCGCACTTATGACCC |
| | | GAAAGGGGGGCTGGACAATGCTAGGCGCCTT |
| | | ACAGGTTTCCATGAAACATCCAGCATCCATG |
| | | AGTTCTCAGCTGGTGTGGCCAACCGTGGTGC |
| | | CAGCATTCGTATCCCCAGGAGCGTGGGCCAA |
| | | GTGAAGAAAGGCTATTTTGAGGACCGCCGAC |
| | | CTTCTGCCAATTGTGACCCTTATGCTGTGACA |
| | | GAGGCACTAGTCCGTACATGTCTCCTCAACG |
| | | AGACTGGGGATGAGCCTTTTGAGTACAAGAA |
| | | C (SEQ ID NO: 5) |
| Green | MATSASSHLSKAIKHMYMKLPQGD | ATGGCCACCTCTGCAAGTTCCCATTTGAGTA |
| anole | KVQAMYIWIDGTGEFLRCKTRTLD | AAGCTATAAAGCATATGTACATGAAGTTGCC |
| (Anolis | HEPKNIEDLPEWNFDGSSTYQSEGS | TCAGGGGGACAAGGTGCAAGCTATGTACATC |
| carolinensis) | NSDMYLVPSAMFRDPFRKDPNKLV | TGGATAGATGGGACTGGCGAGTTTCTGCGTT |
| | LCEVLKYNRKPAETNIRNSCERIMD | GTAAGACTCGGACACTGGATCATGAGCCCAA |
| | MVSNQNPWFGMEQEYTLLGTDGHP | GAACATTGAAGATCTACCAGAATGGAATTTT |
| | FGWPSNGFPGPQGPYYCGVGADKA | GATGGCTCCAGCACCTACCAGTCCGAGGGCT |
| | YGRDIVEAHYRACLYAGVNIGGTN | CCAACAGTGACATGTACCTTGTGCCTTCTGCC |
| | AEVMPAQWEFQVGPCEGIEMGDHL | ATGTTCCGGGACCCTTTCCGCAAAGACCCCA |
| | WIARFILHRVCEDFGVIVSFDPKPIPG | ACAAGCTGGTTCTCTGCGAGGTCTTAAAGTA |
| | NWNGAGCHTNFSTKAMREEGGLK | CAACCGCAAGCCTGCAGAAACAAATATAAG |
| | HIEEAIEKLGKRHQYHIRAYDPKGG | GAACAGCTGTGAAAGAATCATGGATATGGTG |
| | LDNARRLTGFHETSNINEFSAGVAN | TCTAATCAGAATCCCTGGTTCGGGATGGAGC |
| | RGASIRIPRSVGQEKKGYFEDRRPSA | AAGAATATACTCTTCTGGGAACAGATGGACA |
| | NCDPYAVTEALVRTCLLNETGDEPF | TCCCTTTGGCTGGCCTTCCAATGGTTTTCCTG |
| | EYKN | GACCCCAAGGCCCATACTACTGTGGAGTTGG |
| | (SEQ ID NO: 2) | AGCAGACAAGGCTTATGGGCGAGACATTGTG |
| | | GAAGCCCATTATCGAGCCTGCCTATATGCTG |
| | | GTGTGAACATTGGTGGCACAAATGCAGAAGT |
| | | TATGCCAGCGCAGTGGGAGTTCCAGGTGGGT |
| | | CCATGCGAAGGAATTGAGATGGGTGACCACC |
| | | TCTGGATTGCTCGGTTCATCCTTCATAGAGTA |
| | | TGTGAAGACTTCGGTGTCATTGTGTCTTTTGA |
| | | CCCCAAGCCTATCCCAGGCAACTGGAATGGA |
| | | GCTGGATGCCACACGAACTTCAGCACTAAAG |
| | | CCATGCGAGAGGAAGGAGGCCTCAAGCATAT |
| | | TGAAGAAGCTATTGAGAAGCTGGGCAAGCGC |
| | | CATCAGTACCACATCCGTGCCTATGATCCCA |
| | | AAGGGGGGCTGGATAATGCCAGGCGTCTGAC |
| | | CGGGTTCCATGAGACGTCCAACATCAATGAG |
| | | TTCTCTGCTGGAGTAGCCAACCGTGGTGCCA |
| | | GCATCCGCATCCCAAGAAGCGTGGGCCAAGA |
| | | GAAGAAAGGCTACTTTGAAGATCGCCGGCCC |
| | | TCCGCCAACTGTGACCCTTATGCCGTGACCG |
| | | AAGCACTAGTCCGCACTTGTCTCCTCAACGA |
| | | AACCGGGGATGAGCCCTTTGAGTACAAGAAC |
| | | (SEQ ID NO: 6) |
| Spotted | MIKMATSASSSLSKAVKQQYMELP | ATGATCAAGATGGCCACCTCCGCCAGCTCTA |
| gar | QGDKVQAMYIWIDGTGEGLRCKTR | GCCTGAGCAAGGCCGTCAAGCAGCAGTACAT |
| (Lepisosteus | TLDSEPKSIEDLPEWNFDGSSTYQSE | GGAGCTGCCCCAGGGCGACAAGGTGCAGGC |
| oculatus) | GSNSDMYLIPAAMYRDPFRKDPNK | CATGTACATCTGGATCGACGGCACCGGGGAG |
| | LVLCEVLKYNRRPAETNLRSSCKRI | GGGCTGCGCTGCAAGACCCGGACTCTGGACT |
| | MDMVANNRPWFGMEQEYTILGTD | CCGAGCCCAAGAGCATCGAAGACCTGCCCGA |
| | GHPFGWPSNGFPGPQGPYYCGVGA | GTGGAACTTCGACGGCTCCAGCACCTACCAG |
| | DKAYGRDIVEAHYRACLYAGVQIC | TCCGAGGGCTCCAACAGCGACATGTACCTGA |
| | GTNAEVMPAQWEFQVGPSEGIDMG | TCCCGGCGGCCATGTACCGGGACCCCTTCCG |
| | DHLWIARFILHRVCEDFGVVASFDP | CAAGGACCCCAACAAGCTGGTCCTGTGTGAA |
| | KPIPGNWNGAGCHTNFSTKEMREE | GTGCTGAAGTACAACAGGAGGCCTGCAGAG |
| | NGLKYIEESIERLSRRHRYHIRAYDP | ACCAACCTCCGCAGCTCCTGCAAGAGAATCA |
| | KGGLDNARRLTGHNETSNIHEFSAG | TGGACATGGTGGCCAACAATCGCCCCTGGTT |
| | VANRGASIRIPRAVGQDKKGYFEDR | CGGCATGGAGCAGGAGTACACCATACTGGGC |
| | RPSANCDPYSVTEALIRTCLLKEEGD | ACGGACGGCCACCCATTTGGCTGGCCCTCCA |
| | EPVEYKN | ATGGCTTCCCCGGACCCCAGGGTCCCTATTA |
| | (SEQ ID NO: 3) | CTGTGGGGTGGGTGCTGACAAGGCTTATGGG |
| | | CGAGACATTGTAGAAGCTCATTACCGAGCCT |
| | | GCCTGTACGCCGGAGTTCAGATCTGTGGCAC |
| | | CAATGCTGAAGTCATGCCAGCCCAGTGGGAG |
| | | TTCCAGGTTGGCCCCAGTGAAGGAATCGACA |
| | | TGGGAGACCACTTGTGGATTGCCAGGTTCAT |
| | | TCTGCACAGGGTCTGTGAGGACTTCGGAGTC |
| | | GTGGCCTCATTTGACCCCAAACCCATCCCAG |
| | | GGAACTGGAACGGTGCTGGCTGCCACACCAA |
| | | CTTCAGCACCAAGGAGATGAGGGAAGAAAA |
| | | CGGCTTGAAGTACATCGAGGAGTCGATTGAG |
| | | AGGCTGAGCAGGAGACACCGTTACCACATCC |
| | | GTGCCTACGACCCCAAGGGCGGCCTGGACAA |
| | | CGCCAGGCGCCTGACGGGTCACAACGAAACC |
| | | TCCAACATCCACGAGTTCTCCGCGGGCGTGG |
| | | CCAACCGTGGGGCCAGCATCCGCATCCCTCG |
| | | CGCCGTGGGCCAGGACAAGAAGGGCTACTTC |
| | | GAGGACCGGCGCCCCTCCGCCAACTGCGACC |
| | | CCTACAGCGTGACGGAGGCGCTGATCCGCAC |
| | | ATGTTTACTGAAGGAAGAGGGAGATGAACCT |
| | | GTGGAGTACAAGAAC (SEQ ID NO: 7) |
| Chinese | MATSASSHLNKGIKQMYMSLPQGE | ATGGCCACCTCAGCAAGTTCCCACTTGAACA |
| Hamster | KVQAMYIWVDGTGEGLRCKTRTLD | AAGGCATCAAGCAAATGTACATGTCCCTGCC |
| (Cricetulus | CEPKCVEELPEWNFDGSSTFQSESSN | CCAGGGTGAGAAAGTCCAAGCCATGTATATC |
| griseus) | SDMYLSPVAMFRDPFRKEPNKLVFC | TGGGTTGATGGTACCGGAGAAGGACTGCGCT |
| | EVFKYNQKPAETNLRHTCKRIMDM | GCAAAACCCGCACCCTGGACTGTGAGCCCAA |
| | VSNQHPWFGMEQEYTLLGTDGHPF | GTGTGTAGAAGAGTTACCTGAGTGGAATTTT |
| | GWPSDGFPGPQGLYYCGVGADKAY | GATGGCTCTAGTACCTTTCAGTCTGAGAGCTC |
| | RRDIMEAHYRACLYAGVKITGTYA | CAACAGTGACATGTATCTCAGCCCTGTTGCC |
| | EVKHAQWEFQIGPCEGIRMGDHLW | ATGTTTCGGGACCCCTTCCGCAAAGAGCCCA |
| | VARFILHRVCKDFGVIATFDSKPIPG | ACAAGCTGGTGTTCTGTGAAGTCTTCAAGTA |
| | NWNGAGCHTNFSTKTMREENGLKH | CAACCAGAAGCCTGCAGAGACCAATTTAAGA |
| | IKEAIEKLSKRHRYHIRAYDPKGGL | CACACGTGTAAACGGATAATGGACATGGTGA |
| | DNARRLTGFHKTSNINDFSAGVADR | GCAACCAGCACCCCTGGTTTGGAATGGAACA |
| | SASIRIPRTVGQEKKGYFEARCPSAN | GGAGTATACTCTCTTGGGAACAGATGGGCAC |
| | CDPFAVTEAIVRTCLLNETGDQPFQ | CCTTTTGGTTGGCCTTCCGATGGCTTCCCTGG |
| | YKN | GCCCCAAGGTCTGTATTACTGTGGTGTGGGC |
| | (SEQ ID NO: 4) | GCAGACAAAGCCTATCGCAGGGACATCATGG |
| | | AGGCTCACTACCGTGCCTGCTTGTATGCTGG |
| | | GGTCAAGATTACAGGAACATATGCTGAGGTC |
| | | AAGCATGCCCAGTGGGAGTTCCAAATAGGAC |
| | | CCTGTGAAGGAATCCGCATGGGAGATCATCT |
| | | CTGGGTGGCCCGTTTCATCTTGCATCGAGTAT |
| | | GTAAAGACTTTGGAGTAATAGCAACCTTTGA |
| | | CTCCAAGCCCATTCCTGGGAACTGGAATGGT |
| | | GCAGGCTGCCATACCAACTTTAGTACCAAGA |
| | | CCATGCGGGAGGAGAATGGTCTGAAGCACAT |
| | | CAAGGAGGCCATTGAGAAACTAAGCAAGCG |
| | | GCACCGGTACCATATTCGAGCCTACGATCCC |
| | | AAGGGGGGGCTGGACAATGCCCGTCGTCTGA |
| | | CTGGGTTCCACAAAACGTCCAACATCAACGA |
| | | CTTTTCTGCTGGCGTCGCCGACCGCAGTGCCA |
| | | GCATCCGCATTCCCCGGACTGTCGGCCAGGA |
| | | GAAGAAAGGTTACTTTGAAGCCCGCTGCCCC |
| | | TCTGCCAATTGTGACCCCTTTGCAGTGACAG |
| | | AAGCCATCGTCCGCACATGCCTTCTCAATGA |
| | | GACTGGCGACCAGCCCTTCCAATACAAAAAC |
| | | TAA (SEQ ID NO: 8) |

In some embodiments, provided herein are uses of nucleotide sequences encoding an Alligator GS (an Alligator GS-encoding sequence) as selectable markers. An Alligator GS is a GS of Alligator origin. In some embodiments, the Alligator GS is derived from Alligatoridae. In some embodiments, the Alligator GS is derived from Alligator. In some embodiments, the Alligator GS is derived from Alligator mississippiensis. Exemplary amino acid sequences of Alligator GS can be found in the table above and in public database, e.g., Uniprot #A0A151MI87.

Provided herein are uses of Alligator GS-encoding sequences as selectable markers. In some embodiments, the nucleotide sequences encode an Alligator GS having an amino acid sequence comprising or consisting of SEQ ID NO:1. In some embodiments, the Alligator GS is a functional variant of the GS that has the amino acid sequence of SEQ ID NO:1. A functional variant of the GS that has the amino acid sequence of SEQ ID NO:1 maintains the basic structure and the GS activity of the reference GS. The functional variant can have an amino acid sequence that has, for example, about 1 to about 25, about 1 to about 20, about 1 to about 15, about 1 to about 10, or about 1 to about 5 amino acid substitutions, deletions, and/or additions as compared to SEQ ID NO:1. In some embodiments, the amino acid sequence of the functional variants only differ from SEQ ID NO:1 by the presence of at most 22, 20, 15, 10, 9, 8, 7, 6, 5, 4, 3, 2 or 1 amino acid variations, including substitutions, insertions and deletions. The changes to an amino acid sequence can be amino acid substitutions. The changes to an amino acid sequence can be conservative amino acid substitutions. The variants can occur naturally in Alligator species (such as allelic variants or splice variants). Alternatively, the variants can be obtained by genetic engineering. In some embodiments, the functional variant has reduced GS activity compared to a wild-type Alligator GS. In some embodiments, the functional variant with reduced GS activity comprises one or more mutations in the glutamate binding region, the ATP binding region, the ammonia binding region, or any combination thereof. In some embodiments, the functional variant with reduced GS activity comprises one or more mutations in the beta grasp domain, the catalytic domain, or combination thereof.

Provided herein are uses of Alligator GS-encoding sequences as selectable markers. In some embodiments, the nucleotide sequences encode an Alligator GS having an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:1. In some embodiments, the GS has an amino acid sequence that is at least 90% identical to SEQ ID NO: 1. In some embodiments, the GS has an amino acid sequence that is at least 92% identical to SEQ ID NO:1. In some embodiments, the GS has an amino acid sequence that is at least 95% identical to SEQ ID NO:1. In some embodiments, the GS has an amino acid sequence that is at least 98% identical to SEQ ID NO: 1. In some embodiments, the GS has an amino acid sequence that is at least 99% identical to SEQ ID NO:1. In some embodiments, the GS has the amino acid sequence of SEQ ID NO:1. In some embodiments, the Alligator GS is derived from Alligatoridae and has an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:1. In some embodiments, the Alligator GS is derived from Alligator and has an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:1. In some embodiments, the Alligator GS is derived from Alligatoridae and has an amino acid sequence that is at least 95% identical to SEQ ID NO: 1. In some embodiments, the GS provided herein for use as a selectable marker has reduced GS activity compared to a wild-type Alligator GS. In some embodiments, the GS with reduced activity comprises one or more mutations in the glutamate binding region, the ATP binding region, the ammonia binding region, or any combination thereof. In some embodiments, the functional variant with reduced GS activity comprises one or more mutations in the beta grasp domain, the catalytic domain, or combination thereof.

Provided herein are uses of Alligator GS-encoding sequences as selectable markers. In some embodiments, the nucleotide sequences encode a functional fragment of the Alligator GS having the amino acid sequence of SEQ ID NO:1. A functional fragment of the Alligator GS that has the amino acid sequence of SEQ ID NO:1 maintains the GS activity of the reference GS. In some embodiments, the functional fragment comprises at least 100 consecutive amino acids of SEQ ID NO:1. In some embodiments, the functional fragment comprises at least 150 consecutive amino acids of SEQ ID NO:1. In some embodiments, the functional fragment comprises at least 200 consecutive amino acids of SEQ ID NO: 1. In some embodiments, the functional fragment comprises at least 250 consecutive amino acids of SEQ ID NO:1. In some embodiments, the functional fragment comprises at least 300 consecutive amino acids of SEQ ID NO:1. In some embodiments, the functional fragment provided herein for use as a selectable marker has reduced GS activity compared to a wild-type Alligator GS.

As the catalytic domain itself would be sufficient to convey the enhanced performance of the GS marker, in some embodiments, provided herein are GS that comprises a catalytic domain from an Alligator GS. In some embodiments, the catalytic domain of an Alligator GS (e.g., the GS from Alligator mississippiensis) can be comprised of amino acids 134-351 of the protein. In some embodiments, the catalytic domain of an Alligator GS (e.g., the GS from Alligator mississippiensis) can be comprised of amino acids 110-359 of the protein. In some embodiments, the GS comprises a catalytic domain from an Alligator GS having an amino acid sequence that is at least 85%, at least 90%, at least 92%, at least 95%, at least 98%, at least 99%, or 100% identical to SEQ ID NO:1. In some embodiments, the catalytic domain has an amino acid sequence that is at least 85%, at least 90%, at least 92%, at least 95%, at least 98%, at least 99%, or 100% identical to amino acids 110-359 of SEQ ID NO:1. In some embodiments, the catalytic domain has amino acids 110-359 of SEQ ID NO:1. The GS can further comprise the N-terminal region from any other GS. In some embodiments, the GS can further comprise the beta-grasp domain from any other GS. The GS can comprise the N-terminal region (e.g., the beta-grasp domain) from, for example, a hamster GS, a green anole GS, a spotted gar GS, a platypus GS, a turtle GS, a rat GS, an opossum GS, a wombat GS, or a zebra finch GS. In some embodiments, the GS provided herein comprises the catalytic domain from an Alligator GS and the beta-grasp domain from a hamster GS, a green anole GS, a spotted gar GS, a platypus GS, a turtle GS, a rat GS, an opossum GS, a wombat GS, or a zebra finch GS.

Provided herein are uses of Alligator GS-encoding sequences as selectable markers. In some embodiments, the Alligator GS has reduced stability at mRNA level or protein level. In some embodiments, the Alligator GS-encoding sequences provided herein are operatively linked to mRNA destabilizing elements. In some embodiments, the Alligator GS provided herein comprises a degron. The degron can be any degron disclosed herein or otherwise known in the art. In some embodiments, the Alligator GS has a N-terminal degron. In some embodiments, the Alligator GS has a C-terminal degron. In some embodiments, the degron is a PEST sequence (e.g., SEQ ID NO:13). In some embodiments, the degron is an ODD sequence (e.g., SEQ ID NO: 14). In some embodiments, the degron is an IκBα sequence (e.g., SEQ ID NO:15). In some embodiments, the degron has an amino acid sequence selected from the group consisting of SEQ ID NOs:13-15.

In some embodiments, provided herein are uses of nucleotide sequences encoding a green anole GS as selectable markers. A green anole GS is a GS of green anole origin. In some embodiments, the green anole GS is derived from Dactyloidae. In some embodiments, the green anole GS is derived from Anolis. In some embodiments, the green anole GS is derived from Anolis carolinensis. Exemplary amino acid sequences of green anole GS can be found in the table above and in public database, e.g., Uniprot #H9GDW2.

Provided herein are uses of green anole GS-encoding sequences as selectable markers. In some embodiments, the nucleotide sequences encode a green anole GS having an amino acid sequence comprising or consisting of SEQ ID NO:2. In some embodiments, the green anole GS is a functional variant of the GS that has the amino acid sequence of SEQ ID NO:2. A functional variant of the GS that has the amino acid sequence of SEQ ID NO:2 maintains the basic structure and the GS activity of the reference GS. The functional variant can have an amino acid sequence that has, for example, about 1 to about 25, about 1 to about 20, about 1 to about 15, about 1 to about 10, or about 1 to about 5 amino acid substitutions, deletions, and/or additions as compared to SEQ ID NO:2. In some embodiments, the amino acid sequence of the functional variants only differ from SEQ ID NO:2 by the presence of at most 22, 20, 15, 10, 9, 8, 7, 6, 5, 4, 3, 2 or 1 amino acid variations, including substitutions, insertions and deletions. The changes to an amino acid sequence can be amino acid substitutions. The changes to an amino acid sequence can be conservative amino acid substitutions. The variants can occur naturally in green anole species (such as allelic variants or splice variants). Alternatively, the variants can be obtained by genetic engineering. In some embodiments, the functional variant has reduced GS activity compared to a wild-type green anole GS. In some embodiments, the functional variant with reduced GS activity comprises one or more mutations in the glutamate binding region, the ATP binding region, the ammonia binding region, or any combination thereof. In some embodiments, the functional variant with reduced GS activity comprises one or more mutations in the beta grasp domain, the catalytic domain, or combination thereof.

Provided herein are uses of green anole GS-encoding sequences as selectable markers. In some embodiments, the nucleotide sequences encode a green anole GS having an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:2. In some embodiments, the GS has an amino acid sequence that is at least 90% identical to SEQ ID NO:2. In some embodiments, the GS has an amino acid sequence that is at least 92% identical to SEQ ID NO:2. In some embodiments, the GS has an amino acid sequence that is at least 95% identical to SEQ ID NO:2. In some embodiments, the GS has an amino acid sequence that is at least 98% identical to SEQ ID NO:2. In some embodiments, the GS has an amino acid sequence that is at least 99% identical to SEQ ID NO:2. In some embodiments, the GS has the amino acid sequence of SEQ ID NO:2. In some embodiments, the green anole GS is derived from Dactyloidae and has an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:2. In some embodiments, the green anole GS is derived from Anolis and has an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:2. In some embodiments, the green anole GS is derived from Dactyloidae and has an amino acid sequence that is at least 95% identical to SEQ ID NO:2. In some embodiments, the GS provided herein for use as a selectable marker has reduced GS activity compared to a wild-type green anole GS. In some embodiments, the GS with reduced activity comprises one or more mutations in the glutamate binding region, the ATP binding region, the ammonia binding region, or any combination thereof. In some embodiments, the functional variant with reduced GS activity comprises one or more mutations in the beta grasp domain, the catalytic domain, or combination thereof.

Provided herein are uses of green anole GS-encoding sequences as selectable markers. In some embodiments, the nucleotide sequences encode a functional fragment of the green anole GS having the amino acid sequence of SEQ ID NO:2. A functional fragment of the green anole GS that has the amino acid sequence of SEQ ID NO:2 maintains the GS activity of the reference GS. In some embodiments, the functional fragment comprises at least 100 consecutive amino acids of SEQ ID NO:2. In some embodiments, the functional fragment comprises at least 150 consecutive amino acids of SEQ ID NO:2. In some embodiments, the functional fragment comprises at least 200 consecutive amino acids of SEQ ID NO:2. In some embodiments, the functional fragment comprises at least 250 consecutive amino acids of SEQ ID NO:2. In some embodiments, the functional fragment comprises at least 300 consecutive amino acids of SEQ ID NO:2. In some embodiments, the functional fragment provided herein for use as a selectable marker has reduced GS activity compared to a wild-type green anole GS.

As the catalytic domain itself would be sufficient to convey the enhanced performance of the GS marker, in some embodiments, provided herein are GS that comprises a catalytic domain from a green anole GS. In some embodiments, the catalytic domain of a green anole GS (e.g., the GS from Anolis carolinensis) can be comprised of amino acids 134-351 of the protein. In some embodiments, the catalytic domain of a green anole GS (e.g., the GS from Anolis carolinensis) can be comprised of amino acids 110-359 of the protein. In some embodiments, the GS comprises a catalytic domain from a green anole GS having an amino acid sequence that is at least 85%, at least 90%, at least 92%, at least 95%, at least 98%, at least 99%, or 100% identical to SEQ ID NO:2. In some embodiments, the catalytic domain has an amino acid sequence that is at least 85%, at least 90%, at least 92%, at least 95%, at least 98%, at least 99%, or 100% identical to amino acids 110-359 of SEQ ID NO:2. In some embodiments, the catalytic domain has amino acids 110-359 of SEQ ID NO:2. The GS can further comprise the N-terminal region from any other GS. In some embodiments, the GS can further comprise the beta-grasp domain from any other GS. The GS can comprise the N-terminal region (e.g., the beta-grasp domain) from, for example, a hamster GS, an Alligator, a spotted gar GS, a platypus GS, a turtle GS, a rat GS, an opossum GS, a wombat GS, or a zebra finch GS. In some embodiments, the GS provided herein comprises the catalytic domain from a green anole GS and the beta-grasp domain from a hamster GS, an Alligator, a spotted gar GS, a platypus GS, a turtle GS, a rat GS, an opossum GS, a wombat GS, or a zebra finch GS.

Provided herein are uses of green anole GS-encoding sequences as selectable markers. In some embodiments, the green anole GS has reduced stability at mRNA level or protein level. In some embodiments, the green anole GS-encoding sequences provided herein are operatively linked to mRNA destabilizing elements. In some embodiments, the green anole GS provided herein comprises a degron. The degron can be any degron disclosed herein or otherwise known in the art. In some embodiments, the green anole GS has a N-terminal degron. In some embodiments, the green anole GS has a C-terminal degron. In some embodiments, the degron is a PEST sequence (e.g., SEQ ID NO:13). In some embodiments, the degron is an ODD sequence (e.g., SEQ ID NO:14). In some embodiments, the degron is an IκBα sequence (e.g., SEQ ID NO:15). In some embodiments, the degron has an amino acid sequence selected from the group consisting of SEQ ID NOs:13-15.

In some embodiments, provided herein are uses of nucleotide sequences encoding a spotted gar GS as selectable markers. A spotted gar GS is a GS of spotted gar origin. In some embodiments, the spotted gar GS is derived from Lepisosteidae. In some embodiments, the spotted gar GS is derived from Lepisosteus. In some embodiments, a spotted gar GS is derived from Lepisosteus oculatus. Exemplary amino acid sequences of spotted gar GS can be found in the table above and in public database, e.g., Uniprot #W5MAD8.

Provided herein are uses of spotted gar GS-encoding sequences as selectable markers. In some embodiments, the nucleotide sequences encode a spotted gar GS having an amino acid sequence comprising or consisting of SEQ ID NO:3. In some embodiments, the spotted gar GS is a functional variant of the GS that has the amino acid sequence of SEQ ID NO:3. A functional variant of the GS that has the amino acid sequence of SEQ ID NO:3 maintains the basic structure and the GS activity of the reference GS. The functional variant can have an amino acid sequence that has, for example, about 1 to about 25, about 1 to about 20, about 1 to about 15, about 1 to about 10, or about 1 to about 5 amino acid substitutions, deletions, and/or additions as compared to SEQ ID NO:3. In some embodiments, the amino acid sequence of the functional variants only differ from SEQ ID NO:3 by the presence of at most 22, 20, 15, 10, 9, 8, 7, 6, 5, 4, 3, 2 or 1 amino acid variations, including substitutions, insertions and deletions. The changes to an amino acid sequence can be amino acid substitutions. The changes to an amino acid sequence can be conservative amino acid substitutions. The variants can occur naturally in spotted gar species (such as allelic variants or splice variants). Alternatively, the variants can be obtained by genetic engineering. In some embodiments, the functional variant has reduced GS activity compared to a wild-type spotted gar GS. In some embodiments, the functional variant with reduced GS activity comprises one or more mutations in the glutamate binding region, the ATP binding region, the ammonia binding region, or any combination thereof. In some embodiments, the functional variant with reduced GS activity comprises one or more mutations in the beta grasp domain, the catalytic domain, or combination thereof.

Provided herein are uses of spotted gar GS-encoding sequences as selectable markers. In some embodiments, the nucleotide sequences encode a spotted gar GS having an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:3. In some embodiments, the GS has an amino acid sequence that is at least 90% identical to SEQ ID NO:3. In some embodiments, the GS has an amino acid sequence that is at least 92% identical to SEQ ID NO:3. In some embodiments, the GS has an amino acid sequence that is at least 95% identical to SEQ ID NO:3. In some embodiments, the GS has an amino acid sequence that is at least 98% identical to SEQ ID NO:3. In some embodiments, the GS has an amino acid sequence that is at least 99% identical to SEQ ID NO: 3. In some embodiments, the GS has the amino acid sequence of SEQ ID NO:3. In some embodiments, the spotted gar GS is derived from Lepisosteidae and has an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:3. In some embodiments, the spotted gar GS is derived from Lepisosteus and has an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:3. In some embodiments, the spotted gar GS is derived from Lepisosteidae and has an amino acid sequence that is at least 95% identical to SEQ ID NO:3. In some embodiments, the GS provided herein for use as a selectable marker has reduced GS activity compared to a wild-type spotted gar GS. In some embodiments, the GS with reduced activity comprises one or more mutations in the glutamate binding region, the ATP binding region, the ammonia binding region, or any combination thereof. In some embodiments, the functional variant with reduced GS activity comprises one or more mutations in the beta grasp domain, the catalytic domain, or combination thereof.

As the catalytic domain itself would be sufficient to convey the enhanced performance of the GS marker, in some embodiments, provided herein are GS that comprises a catalytic domain from a spotted gar GS. In some embodiments, the catalytic domain of a spotted gar GS (e.g., the GS from Lepisosteus oculatus) can be comprised of amino acids 137-354 of the protein. In some embodiments, the catalytic domain of a spotted gar GS (e.g., the GS from Lepisosteus oculatus) can be comprised of amino acids 113-362 of the protein. In some embodiments, the GS comprises a catalytic domain from a spotted gar GS having an amino acid sequence that is at least 85%, at least 90%, at least 92%, at least 95%, at least 98%, at least 99%, or 100% identical to SEQ ID NO:3. In some embodiments, the catalytic domain has an amino acid sequence that is at least 85%, at least 90%, at least 92%, at least 95%, at least 98%, at least 99%, or 100% identical to amino acids 113-362 of SEQ ID NO:3. In some embodiments, the catalytic domain has amino acids 113-362 of SEQ ID NO:3. The GS can further comprise the N-terminal region from any other GS. In some embodiments, the GS can further comprise the beta-grasp domain from any other GS. The GS can comprise the N-terminal region (e.g., the beta-grasp domain) from, for example, a hamster GS, an Alligator GS, a green anole GS, a platypus GS, a turtle GS, a rat GS, an opossum GS, a wombat GS, or a zebra finch GS. In some embodiments, the GS provided herein comprises the catalytic domain from a spotted gar GS and the beta-grasp domain from a hamster GS, an Alligator GS, a green anole GS, a platypus GS, a turtle GS, a rat GS, an opossum GS, a wombat GS, or a zebra finch GS.

Provided herein are uses of spotted gar GS-encoding sequences as selectable markers. In some embodiments, the nucleotide sequences encode a functional fragment of the spotted gar GS having the amino acid sequence of SEQ ID NO:3. A functional fragment of the spotted gar GS that has the amino acid sequence of SEQ ID NO:3 maintains the GS activity of the reference GS. In some embodiments, the functional fragment comprises at least 100 consecutive amino acids of SEQ ID NO:3. In some embodiments, the functional fragment comprises at least 150 consecutive amino acids of SEQ ID NO: 3. In some embodiments, the functional fragment comprises at least 200 consecutive amino acids of SEQ ID NO:3. In some embodiments, the functional fragment comprises at least 250 consecutive amino acids of SEQ ID NO:3. In some embodiments, the functional fragment comprises at least 300 consecutive amino acids of SEQ ID NO:3. In some embodiments, the functional fragment provided herein for use as a selectable marker has reduced GS activity compared to a wild-type spotted gar GS.

Provided herein are uses of spotted gar GS-encoding sequences as selectable markers. In some embodiments, the spotted gar GS has reduced stability at mRNA level or protein level. In some embodiments, the spotted gar GS-encoding sequences provided herein are operatively linked to mRNA destabilizing elements. In some embodiments, the spotted gar GS provided herein comprises a degron. The degron can be any degron disclosed herein or otherwise known in the art. In some embodiments, the spotted gar GS has a N-terminal degron. In some embodiments, the spotted gar GS has a C-terminal degron. In some embodiments, the degron is a PEST sequence (e.g., SEQ ID NO:13). In some embodiments, the degron is an ODD sequence (e.g., SEQ ID NO:14). In some embodiments, the degron is an IκBα sequence (e.g., SEQ ID NO:15). In some embodiments, the degron has an amino acid sequence selected from the group consisting of SEQ ID NOs:13-15.

In some embodiments, provided herein are uses of nucleotide sequences encoding a chimeric GS (a chimeric GS-encoding sequence) as selectable markers, wherein the chimeric GS comprise fragments that are derived from different species. For example, a chimeric GS can have a first fragment and a second fragment, each independently derived from an Alligator GS, a green anole GS, or a spotted gar GS. In some embodiments, the first and second fragments are derived from two different species. For illustrative purposes, in some embodiments, a chimeric GS can have a fragment derived from an Alligator GS and a fragment derived from a green anole GS. In some embodiments, a chimeric GS can have a fragment derived from an Alligator GS and a fragment derived from a spotted gar GS. In some embodiments, a chimeric GS can have a fragment derived from a green anole GS and a fragment derived from a spotted gar GS. For illustrative purposes, in some embodiments, a chimeric GS can have a fragment derived from an Alligator GS, a fragment derived from a green anole GS, and a fragment derived from a spotted gar. Variations and permutations of the combinations of fragments derived from different species are expressly contemplated herein. A person of ordinary skill in the art would be able to identify the specific fusion structures and confirm their GS activities with routine experimentation using methods disclosed herein.

TABLE 2
sequence alignment of GS from different species
(see FIG.5 for the alignment).

| | Alligator | Green anole | Spotted gar |
|---|---|---|---|
| | (A. mississippiensis; | (A. carolinensis; | (L. oculatus; |
| | SEQ ID NO: 1) | SEQ ID NO: 2) | SEQ ID NO: 3) |
| Chinese | 87.9% | 85.5% | 90.6% |
| hamster | | | |
| (C. griseus; | | | |
| SEQ ID NO: 4) | | | |

As depicted in FIG. 5, an Alligator GS can comprise the amino acid sequence of SEQ ID NO:4 (wild-type hamster GS) with one or more amino acid substitutions at position(s) selected from the following: N10, G12, Q15, S19, V33, G39, C49, C53, V54, E56, S72, S80, V82, E92, F98, Q106, K107, P108, H128, D152, L160, R172, M176, T191, Y194, K198, H199, I206, R213, V220, K230, A236, T237, S240, T260, N265, H269, K271, S278, R282, A287, K305, N308, N310, D311, D318, S320, T328, E332, A339, C341, F349, I355, Q367, and Q370, numbered according to SEQ ID NO:4. In some embodiments, the amino acid substitution(s) can be selected from N10S, G12A, Q15N, S19K, V33I, G39H, C49H, C53S, V54I, E56D, S72G, S80Q, V82A, E92D, F98L, Q106R, K107Q, P108T, H128L, D152N, L160P, R172G, M176V, T191G, Y194N, K198M, H199P, I206V, R213E, V220I, K230E, A236V, T237S, S240P, T260S, N265G, H269Y, K271E, S278G, R282Q, A287T, K305E, N308S, N310H, D311E, D318N, S320G, T328S, E332V, A339D, C341R, F349Y, I355L, Q367E, and Q370E, numbered according to SEQ ID NO:4.

A green anole GS can comprise the amino acid sequence of SEQ ID NO:4 (wild-type hamster GS) with one or more amino acid substitutions at position(s) selected from the following: N10, G12, Q15, S19, E24, V33, G39, C49, C53, V54, E56, F68, S72, S80, V82, E92, F98, F102, Q106, L113, H115, T116, K118, H128, D152, L160, R172, M176, K189, T191, Y194, K198, H199, I206, R213, V220, K230, A236, T237, S240, T260, N265, K271, S278, R282, K305, D311, D318, S320, T328, A339, C341, F349, I355, Q367, and Q370, numbered according to SEQ ID NO:4. In some embodiments, the amino acid substitution(s) can be selected from: N10S, G12A, Q15H, S19K, E24D, V33I, G39F, C49H, C53N, V54I, E56D, F68Y, S72G, S80V, V82S, E92D, F98L, F102L, Q106R, L113I, H115N, T116S, K118E, H128N, D152N, L160P, R172G, M176V, K189N, T191G, Y194N, K198M, H199P, I206V, R213E, V220I, K230E, A236V, T237S, S240P, T260A, N265G, K271E, S278G, R282Q, K305E, D311E, D318N, S320G, T328S, A339D, C341R, F349Y, I355L, Q367E, and Q370E, numbered according to SEQ ID NO:4.

A spotted gar GS can comprise the amino acid sequence of SEQ ID NO:4 (wild-type hamster GS) with one or more amino acid substitutions at position(s) selected from the following: H8, N10, G12, I13, M16, S19, E24, V33, C49, C53, V54, E56, F68, S72, S80, V82, F85, E92, F98, F102, Q106, K107, H115, T116, S125, Q127, H128, L139, D152, L160, R172, M176, K189, T191, Y194, K198, H199, I206, C209, R213, V220, K230, I235, T237, S240, T260, H269, K271, A273, K276, K279, F303, H304, K305, N310, D311, D318, S320, T328, E332, A339, C341, F349, A350, I355, V356, N362, T364, Q367, F369, and Q370, numbered according to SEQ ID NO:4. In some embodiments, the amino acid substitution(s) can be selected from: H8S, N10S, G12A, 113V, M16Q, S19E, E24D, V33I, C49S, C53S, V54I, E56D, F68Y, S72G, S80I, V82A, F85Y, E92D, F98L, F102L, Q106R, K107R, H115S, T116S, S125A, Q127N, H128R, L139I, D152N, L160P, R172G, M176V, K189Q, T191C, Y194N, K198M, H199P, I206V, C209S, R213D, V220I, K230E, I235V, T237S, S240P, T260E, H269Y, K271E, A273S, K276R, K279R, F303H, H304N, K305E, N310H, D311E, D318N, S320G, T328A, E332D, A339D, C341R, F349Y, A350S, I355L, V356I, N362K, T364E, Q367E, F369V, and Q370E, numbered according to SEQ ID NO:4.

In some embodiments, the amino acid sequence of the GS provided herein that can serve as an efficient selectable marker can comprise SEQ ID NO:4 with one or more amino acid substitution(s) at position(s) selected from the following: H8, N10, G12, I13, Q15, M16, S19, E24, V33, G39, C49, C53, V54, E56, F68, S72, S80, V82, F85, E92, F98, F102, Q106, K107, P108, L113, H115, T116, K118, S125, Q127, H128, L139, D152, L160, R172, M176, K189, T191, Y194, K198, H199, I206, C209, R213, V220, K230, I235, A236, T237, S240, T260, N265, H269, K271, A273, K276, S278, K279, R282, A287, F303, H304, K305, N308, N310, D311, D318, S320, T328, E332, A339, C341, F349, A350, I355, V356, N362, T364, Q367, F369, and Q370, numbered according to SEQ ID NO:4.

In some embodiments, the amino acid sequence of the GS provided herein can comprise SEQ ID NO:4 with an amino acid substitution in the beta grasp domain. The amino acid sequence of the GS provided herein can comprise SEQ ID NO:4 with an amino acid substitution at H8. The amino acid substitution can be H8S. The amino acid sequence of the GS provided herein can comprise SEQ ID NO:4 with an amino acid substitution at N10. The amino acid substitution can be NIOS. The amino acid sequence of the GS provided herein can comprise SEQ ID NO:4 with an amino acid substitution at G12. The amino acid substitution can be G12A. The amino acid sequence of the GS provided herein can comprise SEQ ID NO:4 with an amino acid substitution at I13. The amino acid substitution can be I13V. The amino acid sequence of the GS provided herein can comprise SEQ ID NO:4 with an amino acid substitution at Q15. The amino acid substitution can be Q15H. The amino acid substitution can be Q15N. The amino acid sequence of the GS provided herein can comprise SEQ ID NO:4 with an amino acid substitution at M16. The amino acid substitution can be M16Q. The amino acid sequence of the GS provided herein can comprise SEQ ID NO:4 with an amino acid substitution at S19. The amino acid substitution can be S19K. The amino acid substitution can be S19E. The amino acid sequence of the GS provided herein can comprise SEQ ID NO:4 with an amino acid substitution at E24. The amino acid substitution can be E24D. The amino acid sequence of the GS provided herein can comprise SEQ ID NO:4 with an amino acid substitution at V33. The amino acid substitution can be V33I. The amino acid sequence of the GS provided herein can comprise SEQ ID NO:4 with an amino acid substitution at G39. The amino acid substitution can be G39H. The amino acid substitution can be G39F. The amino acid sequence of the GS provided herein can comprise SEQ ID NO:4 with an amino acid substitution at C49. The amino acid substitution can be C49S. The amino acid substitution can be C49H. The amino acid sequence of the GS provided herein can comprise SEQ ID NO:4 with an amino acid substitution at C53. The amino acid substitution can be C53S. The amino acid substitution can be C53N. The amino acid sequence of the GS provided herein can comprise SEQ ID NO:4 with an amino acid substitution at V54. The amino acid substitution can be V54I. The amino acid sequence of the GS provided herein can comprise SEQ ID NO:4 with an amino acid substitution at E56. The amino acid substitution can be E56D. The amino acid sequence of the GS provided herein can comprise SEQ ID NO:4 with an amino acid substitution at F68. The amino acid substitution can be F68Y. The amino acid sequence of the GS provided herein can comprise SEQ ID NO:4 with an amino acid substitution at S72. The amino acid substitution can be S72G. The amino acid sequence of the GS provided herein can comprise SEQ ID NO:4 with an amino acid substitution at S80. The amino acid substitution can be S80Q. The amino acid substitution can be S80V. The amino acid substitution can be S80I. The amino acid sequence of the GS provided herein can comprise SEQ ID NO:4 with an amino acid substitution at V82. The amino acid substitution can be V82A. The amino acid substitution can be V82S. The amino acid sequence of the GS provided herein can comprise SEQ ID NO:4 with an amino acid substitution at F85. The amino acid substitution can be F85Y. The amino acid sequence of the GS provided herein can comprise SEQ ID NO:4 with an amino acid substitution at E92. The amino acid substitution can be E92D. The amino acid sequence of the GS provided herein can comprise SEQ ID NO:4 with an amino acid substitution at F98. The amino acid substitution can be F98L. The amino acid sequence of the GS provided herein can comprise SEQ ID NO:4 with an amino acid substitution at F102. The amino acid substitution can be F102L.

The amino acid sequence of the GS provided herein can comprise SEQ ID NO:4 with an amino acid substitution at Q106. The amino acid substitution can be Q106R. The amino acid sequence of the GS provided herein can comprise SEQ ID NO:4 with an amino acid substitution at K107. The amino acid substitution can be K107Q. The amino acid substitution can be K107R. The amino acid sequence of the GS provided herein can comprise SEQ ID NO:4 with an amino acid substitution at P108. The amino acid substitution can be P108T. The amino acid sequence of the GS provided herein can comprise SEQ ID NO:4 with an amino acid substitution at H115. The amino acid substitution can be H115N. The amino acid substitution can be H115S. The amino acid sequence of the GS provided herein can comprise SEQ ID NO:4 with an amino acid substitution at T116. The amino acid substitution can be T116S. The amino acid sequence of the GS provided herein can comprise SEQ ID NO:4 with an amino acid substitution at K118. The amino acid substitution can be K118E. The amino acid sequence of the GS provided herein can comprise SEQ ID NO:4 with an amino acid substitution at S125. The amino acid substitution can be S125A. The amino acid sequence of the GS provided herein can comprise SEQ ID NO:4 with an amino acid substitution at Q127. The amino acid substitution can be Q127N. The amino acid sequence of the GS provided herein can comprise SEQ ID NO:4 with an amino acid substitution at H128. The amino acid substitution can be H128L. The amino acid substitution can be H128N. The amino acid substitution can be H128R.

In some embodiments, the amino acid sequence of the GS provided herein can comprise SEQ ID NO:4 with an amino acid substitution in the catalytic domain. The amino acid sequence of the GS provided herein can comprise SEQ ID NO:4 with an amino acid substitution at L139. The amino acid substitution can be L140I. The amino acid sequence of the GS provided herein can comprise SEQ ID NO:4 with an amino acid substitution at D152. The amino acid substitution can be D152N. The amino acid sequence of the GS provided herein can comprise SEQ ID NO:4 with an amino acid substitution at L160. The amino acid substitution can be L160P. The amino acid sequence of the GS provided herein can comprise SEQ ID NO:4 with an amino acid substitution at R172. The amino acid substitution can be R172G. The amino acid sequence of the GS provided herein can comprise SEQ ID NO:4 with an amino acid substitution at M176. The amino acid substitution can be M176V. The amino acid sequence of the GS provided herein can comprise SEQ ID NO:4 with an amino acid substitution at K189. The amino acid substitution can be K189N. The amino acid substitution can be K189Q. The amino acid sequence of the GS provided herein can comprise SEQ ID NO:4 with an amino acid substitution at T191. The amino acid substitution can be T191C. The amino acid substitution can be T191G. The amino acid sequence of the GS provided herein can comprise SEQ ID NO:4 with an amino acid substitution at Y194. The amino acid substitution can be Y194N. The amino acid sequence of the GS provided herein can comprise SEQ ID NO:4 with an amino acid substitution at K198. The amino acid substitution can be K198M. The amino acid sequence of the GS provided herein can comprise SEQ ID NO:4 with an amino acid substitution at H199. The amino acid substitution can be H199P. The amino acid sequence of the GS provided herein can comprise SEQ ID NO:4 with an amino acid substitution at I206. The amino acid substitution can be I206V. The amino acid sequence of the GS provided herein can comprise SEQ ID NO:4 with an amino acid substitution at C209. The amino acid substitution can be C209S. The amino acid sequence of the GS provided herein can comprise SEQ ID NO:4 with an amino acid substitution at R213. The amino acid substitution can be R213E. The amino acid substitution can be R213D. The amino acid sequence of the GS provided herein can comprise SEQ ID NO:4 with an amino acid substitution at V220. The amino acid substitution can be V220I. The amino acid sequence of the GS provided herein can comprise SEQ ID NO:4 with an amino acid substitution at K230. The amino acid substitution can be K230E. The amino acid sequence of the GS provided herein can comprise SEQ ID NO:4 with an amino acid substitution at I235. The amino acid substitution can be I235V. The amino acid sequence of the GS provided herein can comprise SEQ ID NO:4 with an amino acid substitution at A236. The amino acid substitution can be A236V. The amino acid sequence of the GS provided herein can comprise SEQ ID NO:4 with an amino acid substitution at T237. The amino acid substitution can be T237S. The amino acid sequence of the GS provided herein can comprise SEQ ID NO:4 with an amino acid substitution at S240. The amino acid substitution can be S240P. The amino acid sequence of the GS provided herein can comprise SEQ ID NO:4 with an amino acid substitution at T260. The amino acid substitution can be T260S. The amino acid substitution can be T260A. The amino acid substitution can be T260E. The amino acid sequence of the GS provided herein can comprise SEQ ID NO:4 with an amino acid substitution at N265. The amino acid substitution can be N265G. The amino acid sequence of the GS provided herein can comprise SEQ ID NO:4 with an amino acid substitution at H269. The amino acid substitution can be H269Y. The amino acid sequence of the GS provided herein can comprise SEQ ID NO:4 with an amino acid substitution at K271. The amino acid substitution can be K271E. The amino acid sequence of the GS provided herein can comprise SEQ ID NO:4 with an amino acid substitution at A273. The amino acid substitution can be A273S. The amino acid sequence of the GS provided herein can comprise SEQ ID NO:4 with an amino acid substitution at K276. The amino acid substitution can be K276R. The amino acid sequence of the GS provided herein can comprise SEQ ID NO:4 with an amino acid substitution at S278. The amino acid substitution can be S278G. The amino acid sequence of the GS provided herein can comprise SEQ ID NO:4 with an amino acid substitution at K279. The amino acid substitution can be K279R. The amino acid sequence of the GS provided herein can comprise SEQ ID NO:4 with an amino acid substitution at R282. The amino acid substitution can be R282Q. The amino acid sequence of the GS provided herein can comprise SEQ ID NO:4 with an amino acid substitution at A287. The amino acid substitution can be A287T. The amino acid sequence of the GS provided herein can comprise SEQ ID NO:4 with an amino acid substitution at F303. The amino acid substitution can be F303H. The amino acid sequence of the GS provided herein can comprise SEQ ID NO:4 with an amino acid substitution at H304. The amino acid substitution can be H304N. The amino acid sequence of the GS provided herein can comprise SEQ ID NO:4 with an amino acid substitution at K305. The amino acid substitution can be K305E. The amino acid sequence of the GS provided herein can comprise SEQ ID NO:4 with an amino acid substitution at N308. The amino acid substitution can be N308S. The amino acid sequence of the GS provided herein can comprise SEQ ID NO:4 with an amino acid substitution at N310. The amino acid substitution can be N310H. The amino acid sequence of the GS provided herein can comprise SEQ ID NO:4 with an amino acid substitution at D311. The amino acid substitution can be D311E. The amino acid sequence of the GS provided herein can comprise SEQ ID NO:4 with an amino acid substitution at D318. The amino acid substitution can be D318N. The amino acid sequence of the GS provided herein can comprise SEQ ID NO:4 with an amino acid substitution at S320. The amino acid substitution can be S320G. The amino acid sequence of the GS provided herein can comprise SEQ ID NO:4 with an amino acid substitution at T328. The amino acid substitution can be T328S. The amino acid substitution can be T328A. The amino acid sequence of the GS provided herein can comprise SEQ ID NO:4 with an amino acid substitution at E332. The amino acid substitution can be E332D. The amino acid substitution can be E332V. The amino acid sequence of the GS provided herein can comprise SEQ ID NO:4 with an amino acid substitution at A339. The amino acid substitution can be A339D. The amino acid sequence of the GS provided herein can comprise SEQ ID NO:4 with an amino acid substitution at C341. The amino acid substitution can be C341R. The amino acid sequence of the GS provided herein can comprise SEQ ID NO:4 with an amino acid substitution at F349. The amino acid substitution can be F349Y. The amino acid sequence of the GS provided herein can comprise SEQ ID NO:4 with an amino acid substitution at A350. The amino acid substitution can be A350S.

The amino acid sequence of the GS provided herein can comprise SEQ ID NO:4 with an amino acid substitution at I355. The amino acid substitution can be I355L. The amino acid sequence of the GS provided herein can comprise SEQ ID NO:4 with an amino acid substitution at V356. The amino acid substitution can be V356I. The amino acid sequence of the GS provided herein can comprise SEQ ID NO:4 with an amino acid substitution at N362. The amino acid substitution can be N362K. The amino acid sequence of the GS provided herein can comprise SEQ ID NO:4 with an amino acid substitution at T364. The amino acid substitution can be T364E. The amino acid sequence of the GS provided herein can comprise SEQ ID NO:4 with an amino acid substitution at Q367. The amino acid substitution can be Q367E. The amino acid sequence of the GS provided herein can comprise SEQ ID NO:4 with an amino acid substitution at F369. The amino acid substitution can be F369V. The amino acid sequence of the GS provided herein can comprise SEQ ID NO:4 with an amino acid substitution at Q370. The amino acid substitution can be Q370E.

In some embodiments, the GS provided herein that can serve as an efficient selectable marker can have an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% identical to SEQ ID NO:4, with one or more amino acid substitutions at position(s) selected from the following: H8, N10, G12, I13, Q15, M16, S19, E24, V33, G39, C49, C53, V54, E56, F68, S72, S80, V82, F85, E92, F98, F102, Q106, K107, P108, L113, H115, T116, K118, S125, Q127, H128, L139, D152, L160, R172, M176, K189, T191, Y194, K198, H199, I206, C209, R213, V220, K230, I235, A236, T237, S240, T260, N265, H269, K271, A273, K276, S278, K279, R282, A287, F303, H304, K305, N308, N310, D311, D318, S320, T328, E332, A339, C341, F349, A350, I355, V356, N362, T364, Q367, F369, and Q370, numbered according to SEQ ID NO:4. In some embodiments, the amino acid sequence of the GS provided herein can comprise SEQ ID NO:4 with about 1, about 3, about 5, about 10, about 15, about 20, about 25, about 30, about 35, about 40, about 45, about 50, about 55, about 60, about 65, about 70, about 75, or about 80 amino acid substitutions. In some embodiments, the amino acid substitution(s) are selected from H8S, NIOS, G12A, 113V, Q15N, Q15H, M16Q, S19E, S19K, E24D, V33I, G39F, G39H, C49S, C49H, C53S, C53N, V54I, E56D, F68Y, S72G, S80Q, S80V, S80I, V82A, V82S, F85Y, E92D, F98L, F102L, Q106R, K107Q, K107R, P108T, L113I, H115N, H115S, T116S, K118E, S125A, Q127N, H128N, H128L, H128R, L139I, D152N, L160P, R172G, M176V, K189Q, K189N, T191C, T191G, Y194N, K198M, H199P, I206V, C209S, R213E, R213D, V220I, K230E, I235V, A236V, T237S, S240P, T260A, T260S, T260E, N265G, H269Y, K271E, A273S, K276R, S278G, K279R, R282Q, A287T, F303H, H304N, K305E, N308S, N310H, D311E, D318N, S320G, T328S, T328A, E332D, E332V, A339D, C341R, F349Y, A350S, I355L, V356I, N362K, T364E, Q367E, F369V, and Q370E, numbered according to SEQ ID NO:4.

In some embodiments, the GS provided herein can have an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% identical to SEQ ID NO:4, with one or more amino acid substitution(s) at the following positions: N10, G12, S19, V33, C49, C53, V54, E56, S72, S80, V82, E92, F98, Q106, H128, D152, L160, R172, M176, T191, Y194, K198, H199, I206, R213, V220, K230, T237, S240, T260, K271, K305, D311, D318, S320, T328, A339, C341, F349, I355, Q367, and Q370. In some embodiments, the amino acid sequence of the GS provided herein can comprise SEQ ID NO:4 with about 3, about 5, about 10, about 15, about 20, about 25, about 30, about 35, or about 40 amino acid substitutions at the following positions: N10, G12, S19, V33, C49, C53, V54, E56, S72, S80, V82, E92, F98, Q106, H128, D152, L160, R172, M176, T191, Y194, K198, H199, I206, R213, V220, K230, T237, S240, T260, K271, K305, D311, D318, S320, T328, A339, C341, F349, I355, Q367, and Q370. In some embodiments, the amino acid sequence of the GS provided herein can comprise SEQ ID NO:4 with about 1 amino acid substitution. In some embodiments, the amino acid sequence of the GS provided herein can comprise SEQ ID NO:4 with about 3 amino acid substitutions. In some embodiments, the amino acid sequence of the GS provided herein can comprise SEQ ID NO:4 with about 5 amino acid substitutions. In some embodiments, the amino acid sequence of the GS provided herein can comprise SEQ ID NO:4 with about 10 amino acid substitutions. In some embodiments, the amino acid sequence of the GS provided herein can comprise SEQ ID NO:4 with about 15 amino acid substitutions. In some embodiments, the amino acid sequence of the GS provided herein can comprise SEQ ID NO:4 with about 20 amino acid substitutions. In some embodiments, the GS provided herein that can serve as an efficient selectable marker can have the amino acid sequence of SEQ ID NO:4 with amino acid substitutions at each of the following positions of SEQ ID NO:4: N10, G12, S19, V33, C49, C53, V54, E56, S72, S80, V82, E92, F98, Q106, H128, D152, L160, R172, M176, T191, Y194, K198, H199, I206, R213, V220, K230, T237, S240, T260, K271, K305, D311, D318, S320, T328, A339, C341, F349, I355, Q367, and Q370. In some embodiments, the amino acid substitutions are selected from: N10S, G12A, S19K, S19E, V33I, C49H, C49S, C53S, C53N, V54I, E56D, S72G, S80Q, S80V, S80I, V82A, V82S, E92D, F98L, Q106R, H128L, H128N, H128R, D152N, L160P, R172G, M176V, T191G, T191C, Y194N, K198M, H199P, I206V, R213E, R213D, V220I, K230E, T237S, S240P, T260S, T260A, T260E, K271E, K305E, D311E, D318N, S320G, T328S, T328A, A339D, C341R, F349Y, I355L, Q367E, and Q370E.

Some GS disclosed herein as selectable markers have reduced activity or stability (at mRNA or protein level). The reduction in activity or stability in the selectable marker results in more stringent selection, because to grow under such conditions, higher transcriptional activity or higher copy number of the expression cassettes is required. In some embodiments, provided herein are functional variant or functional fragment of Alligator, green anole, or spotted gar GS that has reduced GS activity compared to their wild-type counterpart. As used herein, the term “reduced activity” refers to the decrease in the ability of a variant or fragment of an enzyme (such as GS) in carrying out its enzymatic activity (such as GS activity) when compared to its wild-type counterpart. The activity of an enzyme having reduced activity is about 90%, or about 80%, or about 70%, or about 60%, or about 50%, or about 40%, or about 30%, or about 20%, or about 10%, or about 9%, or about 8%, or about 7%, or about 6%, or about 5%, or about 4%, or about 3%, or about 2%, or about 1% of the activity of a wild-type enzyme. In other words, if the activity of a wild-type enzyme is considered as 100%, the activity of the mutated enzyme (or the enzyme having reduced activity) is reduced by about 10%, or about 20%, or about 30%, or about 40%, or about 50%, or about 60%, or about 70%, or about 80%, or about 90%, or about 91%, or about 92%, or about 93%, or about 94%, or about 95%, or about 96%, or about 97%, or about 98%, or about 99% when compared to the activity of its wild-type counterpart. For example, in some embodiments, the GS with “reduced activity” can have about 20%, about 30%, about 40%, about 50%, about 60%, about 70%, about 80%, or about 90% GS activity as compared to its wild-type counterpart.

In some embodiments, GS disclosed herein that are used as selectable markers have reduced stability at mRNA level. In some embodiments, GS-encoding sequences disclosed herein are operatively linked to an mRNA destabilizing element. In some embodiments, GS-encoding sequences disclosed herein are operatively linked to two or more mRNA destabilizing elements. A key element in mRNA stability is ribonuclear protein (RNP) composition—for example, binding by polyA binding protein (PABP) at the 3′-end and the cap-binding protein eIF4E at the 5′-end are necessary for cytoplasmic mRNA stability. Multiple other RNA-binding proteins (RBPs) have been found to bind within the 5′- or 3′-untranslated regions (UTRs) to dynamically regulate mRNA stability under various cellular conditions, notably at instability-promoting sites such as adenylate-uridylate-rich elements (AU-rich elements; AREs). 3′-UTRs also commonly harbor binding sites for microRNAs, which typically accelerate mRNA decay by recruiting decay machinery and stripping stabilizing mRNP components. (Koh, et al, Sci Rep 9, 5976 (2019); Forrest et al, (2020) PLoS ONE 15(2): e0228730.) Additionally, a stem-loop destabilizing element (SLDE) was found to enhance mRNA decay independently of the nearby ARE (Putland et al. Molecular and Cellular Biology, 22(6): 1664-73 (2002)). In some embodiments, GS-encoding sequences disclosed are operatively linked to an ARE in the 3′-UTR. In some embodiments, GS-encoding sequences disclosed herein are operatively linked two or more copies of ARE in the 3′-UTR. In some embodiments, GS-encoding sequences disclosed are operatively linked to a SLDE in the 3′-UTR. In some embodiments, GS-encoding sequences disclosed are operatively linked to an ARE and a SLDE in the 3′-UTR.

In some embodiments, GS disclosed herein that are used as selectable markers have reduced stability at protein level. In some embodiments, the GS provided herein comprises a degron. In some embodiments, the GS provided herein comprises two or more degrons. The intracellular protein degradation is mediated largely by the ubiquitin (Ub)-proteasome system (UPS). A Ub ligase recognizes a substrate protein through its degradation signal (i.e., degron) and conjugates Ub, a 9-kDa protein (usually in the form of a poly-Ub chain), to an amino acid residue (usually an internal lysine) of the targeted substrate, thereby targeting it for degradation by proteosome. (Varshavsky, PNAS(2019) 116(2):358-366; Timms; Biochem Soc Trans. 2020 48(4): 1557-1567.) Degrons can be present at the N-terminus (N-degron), C-terminus (C-degron), or an internal site of a target protein. Accordingly, in some embodiments, a GS comprising a degron can have the degron fused to its N-terminus or C-terminus. In some embodiments, the nucleotide sequence encoding the platypus GS provided herein is linked to a nucleotide sequence encoding a degron at its 5′ end or 3′ end, such that the degron is fused to the N-terminus or C-terminus of the GS. In some embodiments, the GS provided herein comprises an Arg/N-degron. In some embodiments, the GS provided herein comprises an Ac/N-degron. In some embodiments, the GS provided herein comprises an fMet/N-degron. In some embodiments, the GS provided herein comprises a Pro/N-degron. In some embodiments, the GS provided here comprises a degron having a synthetic degron, which are usually non-natural short peptide having 5-30 amino acids. In some embodiments, the GS provided here comprises a PEST degron (a peptide sequence that is rich in proline (P), glutamic acid (E), serine (S), and threonine (T)). In some embodiments, the GS provided here comprises a PEST degron from ornithine decarboxylase: SHGFPPEVEEQAAGTLPMSCAQESGMDRHPAACASARINV (SEQ ID NO:13). In some embodiments, the GS provided here comprises an ODD (oxygen dependent degradation) domain from the transcription factor HIF1a aa530-652: EFKLELVEKLFAEDTEAKNPFSTQDTDLDLEMLAPYIPMDDDFQLRSFDQLSPLESSSASPES ASPQSTVTVFQQTQIQEPTANATTTTATTDELKTVTKDRMEDIKILIASPSPTHIHKETT (SEQ ID NO:14). In some embodiments, the GS provided here comprises a IkappaBalpha (IκBα) degron: IQQQLGQLTLENLQMLPESEDEESYDTESEFTEFTEDELPYDDCVFGGQR (SEQ ID NO:15).

As such, provided herein are uses of nucleotide sequences encoding an Alligator, green anole, or spotted gar GS disclosed herein, or their functional variant or fragments, as selectable markers. In some embodiments, the selectable markers can be used for the identification of genomic loci for integrating an expression cassette. The genomic loci are selected for their high transcriptional activity. In some embodiments, the selectable markers can be used for the identification of cell clones capable of producing a POI. The cell clones are selected for their high productivity of the POI. In some embodiments, the selectable markers are used in recombinant production of a POI. The POI is described further in sections below. For example, the POI can be selected from the group consisting of an antibody, an enzyme, a soluble protein, a secreted protein, a membrane protein, and a fusion protein. In some embodiments, the POI is recombinantly produced in a mammalian cell. In some embodiments, the POI is recombinantly produced by a CHO cell.

### 5.2 Vectors

Provided herein are also deoxyribonucleic acid (DNA) vectors comprising a nucleotide sequence encoding a GS (a GS-encoding sequence), wherein the GS is an Alligator GS, a green anole GS, or a spotted gar GS. In some embodiments, vectors provided herein are suitable for recombinant production and comprise an expression cassette. In some embodiments, vectors provided herein are suitable for genomic integration.

The term “vector,” as used herein and understood in the art, refers to a vehicle that is used to carry genetic material (e.g., a nucleotide sequence), which can be introduced into a host cell, where it can be replicated and/or expressed. Vectors applicable for use include, for example, expression vectors, plasmids, phage vectors, viral vectors, episomes and artificial chromosomes. The vectors can include one or more selectable marker genes and appropriate expression control sequences. Selectable marker genes can be included, for example, to provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. DNA regions (such as control elements and protein encoding sequences) can be “operatively linked” when they are functionally related to each other. For example, a promoter is operatively linked to a coding sequence if it controls the transcription of the sequence; or a ribosome binding site is operatively linked to a coding sequence if it is positioned so as to permit translation.

An “expression cassette,” as used herein and understood in the art, is a distinct and continuous component of vector DNA, which includes regulatory sequences that can control the expression of a nucleotide sequence potentially carried by the expression cassette. The regulatory sequences include, for example, transcriptional initiation (promoter) and termination sequences, enhancer, intron, origin of replication sites, polyadenylation sequences, peptide signal and chromatin insulator elements. Regulatory sequences are described, for example, in Goeddel, GENE EXPRESSION TECHNOLOGY: METHODS IN ENZYMOLOGY 185, Academic Press, San Diego, Calif (1990). Simply put, the expression cassette directs the host cell's machinery to make RNA and protein(s) encoded by the nucleotide sequence contained in the cassette. Thus, expression in cells from different organisms or species, such as bacteria, yeast, plants, and mammalian cells, requires different regulatory sequences. Vectors provided herein can have one or more expression cassette. The expression cassette can be “empty,” which contains a multiple cloning site (MCS) for inserting a nucleotide sequence encoding a POI (a POI-encoding sequence). The expression cassette can be loaded, which contains a POI-encoding sequence. A “multiple cloning site” or “MCS,” as used herein and understood in the art, refers to a short segment of DNA on a vector which contains multiple restriction sites to allow the insertion of a POI-encoding sequence.

In some embodiments, an expression cassette can include one POI-encoding nucleotide sequence. In some embodiments, an expression can have more than one POI-encoding nucleotide sequences, i.e., multicistronic. A multicistronic expression cassette comprises more than one cistrons, which can be transcribed into an mRNA that simultaneously expresses two or more separate polypeptides. In some embodiments, an expression cassette can be bicistronic, namely, comprising two cistrons. An mRNA transcribed from a bicistronic expression cassette can simultaneously express two separate polypeptides. In some embodiments, a tricistronic expression cassette can be tricistronic, namely, comprising three cistrons. An mRNA transcribed from a tricistronic expression vector can simultaneously express three separate polypeptides.

Cistrons within one expression cassette can be separated by, for example, an internal ribosomal entry site (IRES) or 2A element. An IRES, as understood in the art, refers to nucleotide sequences in an expression cassette which when transcribed into mRNA, can recruit ribosomes directly, without a previous scanning of untranslated region of mRNA by the ribosomes. A 2A element, as understood in the art, encoding self-cleaving short peptides (about 20 amino acids) that provide a mechanism for subsequent separation of equimolarly produced polypeptides of interest. Illustrative 2A self-cleaving peptides include P2A, E2A, F2A, and T2A (see table below).

| | Amino Acid Sequences |
|---|---|
| P2A | (GSG)ATNFSLLKQAGDVEENPGP (SEQ ID NO: 9) |
| E2A | (GSG)QCTNYALLKLAGDVESNPGP (SEQ ID NO: 10) |
| F2A | (GSG)VKQTLNFDLLKLAGDVESNPGP (SEQ ID NO: 11) |
| T2A | (GSG)EGRGSLLTCGDVEENPGP (SEQ ID NO: 12) |

The DNA vectors provided herein comprise a nucleotide sequence encoding a GS that is any Alligator GS, green anole GS, or spotted gar GS described herein. The nucleotide sequence encoding such a GS can be the naturally occurring nucleotide sequence. Alternatively, the triplet codons of the nucleotide sequence encoding such a GS can be optimized for expression in specific host cells, such as CHO cells. Software and algorithms for codon optimization are known in the art, including, for example, the algorithm described in Raab et al. (2010, Syst Synth Biol. 4:215-25).

In some embodiments, provided herein are DNA vectors having a nucleotide sequence encoding a GS that is an Alligator GS (an Alligator GS-encoding sequence). The Alligator GS can be any Alligator GS described herein. In some embodiments, the Alligator GS is derived from Alligatoridae. In some embodiments, the Alligator GS is derived from Alligator. In some embodiments, the Alligator GS is derived from Alligator mississippiensis. In some embodiments, the Alligator GS has an amino acid sequence that is at least 90%, at least 92%, at least 95%, at least 98%, at least 99%, or 100% identical to SEQ ID NO:1. In some embodiments, the GS has the amino acid sequence of SEQ ID NO:1. In some embodiments, the Alligator GS is a functional variant of the GS having the amino acid sequence of SEQ ID NO:1. In some embodiments, the Alligator GS has an amino acid sequence that is at least 95% identical to SEQ ID NO:1. In some embodiments, the Alligator GS is a functional fragment of the GS having the amino acid sequence of SEQ ID NO:1. In some embodiments, the Alligator GS has an amino acid sequence comprising at least 100 consecutive amino acids of SEQ ID NO:1. In some embodiments, the Alligator GS has reduced activity compared to a wild-type Alligator GS. In some embodiments, the Alligator GS has reduced stability at mRNA level or protein level. In some embodiments, the Alligator GS-encoding sequences are operatively linked to mRNA destabilizing elements. In some embodiments, the Alligator GS comprises a degron.

In some embodiments, provided herein are DNA vectors having a nucleotide sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, identical to SEQ ID NO:5. In some embodiments, the nucleotide sequence encoding the Alligator GS is at least 80% identical to SEQ ID NO:5. In some embodiments, the nucleotide sequence encoding the Alligator GS is at least 85% identical to SEQ ID NO:5. In some embodiments, the nucleotide sequence encoding the Alligator GS is at least 90% identical to SEQ ID NO:5. In some embodiments, the nucleotide sequence encoding the Alligator GS is at least 95% identical to SEQ ID NO:5. In some embodiments, the nucleotide sequence encoding the Alligator GS is identical to SEQ ID NO: 5. In some embodiments, the nucleotide sequence encoding an Alligator GS is optimized for expression by a particular host cell, e.g., a CHO cell.

In some embodiments, provided herein are DNA vectors having a nucleotide sequence encoding a GS that is a green anole GS (a green anole GS-encoding sequence). The green anole GS can be any green anole GS described herein. In some embodiments, the green anole GS is derived from Dactyloidae. In some embodiments, the green anole GS is derived from Anolis. In some embodiments, the green anole GS is derived from Anolis carolinensis. In some embodiments, the green anole GS has an amino acid sequence that is at least 90%, at least 92%, at least 95%, at least 98%, at least 99%, or 100% identical to SEQ ID NO:2. In some embodiments, the GS has the amino acid sequence of SEQ ID NO:2. In some embodiments, the green anole GS is a functional variant of the GS having the amino acid sequence of SEQ ID NO:2. In some embodiments, the green anole GS has an amino acid sequence that is at least 95% identical to SEQ ID NO:2. In some embodiments, the green anole GS is a functional fragment of the GS having the amino acid sequence of SEQ ID NO:2. In some embodiments, the green anole GS has an amino acid sequence comprising at least 100 consecutive amino acids of SEQ ID NO:2. In some embodiments, the green anole GS has reduced activity compared to a wild-type green anole GS. In some embodiments, the green anole GS has reduced stability at mRNA level or protein level. In some embodiments, the green anole GS-encoding sequences are operatively linked to mRNA destabilizing elements. In some embodiments, the green anole GS comprises a degron.

In some embodiments, provided herein are DNA vectors having a nucleotide sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, identical to SEQ ID NO:6. In some embodiments, the nucleotide sequence encoding the green anole GS is at least 80% identical to SEQ ID NO:6. In some embodiments, the nucleotide sequence encoding the green anole GS is at least 85% identical to SEQ ID NO:6. In some embodiments, the nucleotide sequence encoding the green anole GS is at least 90% identical to SEQ ID NO:6. In some embodiments, the nucleotide sequence encoding the green anole GS is at least 95% identical to SEQ ID NO:6. In some embodiments, the nucleotide sequence encoding the green anole GS is identical to SEQ ID NO:6. In some embodiments, the nucleotide sequence encoding a green anole GS is optimized for expression by a particular host cell, e.g., a CHO cell.

In some embodiments, provided herein are DNA vectors having a nucleotide sequence encoding a GS that is a spotted gar GS (a spotted gar GS-encoding sequence). The spotted gar GS can be any spotted gar GS described herein. In some embodiments, the spotted gar GS is derived from Lepisosteidae. In some embodiments, the spotted gar GS is derived from Lepisosteus. In some embodiments, the spotted gar GS is derived from Lepisosteus oculatus. In some embodiments, the spotted gar GS has an amino acid sequence that is at least 90%, at least 92%, at least 95%, at least 98%, at least 99%, or 100% identical to SEQ ID NO:3. In some embodiments, the GS has the amino acid sequence of SEQ ID NO:3. In some embodiments, the spotted gar GS is a functional variant of the GS having the amino acid sequence of SEQ ID NO:3. In some embodiments, the spotted gar GS has an amino acid sequence that is at least 95% identical to SEQ ID NO:3. In some embodiments, the spotted gar GS is a functional fragment of the GS having the amino acid sequence of SEQ ID NO:3. In some embodiments, the spotted gar GS has an amino acid sequence comprising at least 100 consecutive amino acids of SEQ ID NO:3. In some embodiments, the spotted gar GS has reduced activity compared to a wild-type spotted gar GS. In some embodiments, the spotted gar GS has reduced stability at mRNA level or protein level. In some embodiments, the spotted gar GS-encoding sequences are operatively linked to mRNA destabilizing elements. In some embodiments, the spotted gar GS comprises a degron.

In some embodiments, provided herein are DNA vectors having a nucleotide sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, identical to SEQ ID NO:7. In some embodiments, the nucleotide sequence encoding the spotted gar GS is at least 80% identical to SEQ ID NO:7. In some embodiments, the nucleotide sequence encoding the spotted gar GS is at least 85% identical to SEQ ID NO:7. In some embodiments, the nucleotide sequence encoding the spotted gar GS is at least 90% identical to SEQ ID NO:7. In some embodiments, the nucleotide sequence encoding the spotted gar GS is at least 95% identical to SEQ ID NO:7. In some embodiments, the nucleotide sequence encoding the spotted gar GS is identical to SEQ ID NO:7. In some embodiments, the nucleotide sequence encoding a spotted gar GS is optimized for expression by a particular host cell, e.g., a CHO cell.

In some embodiments, vectors provided herein are suitable for production of a recombinant POI. The vectors provided herein comprise any GS-encoding sequence disclosed herein as well as an expression cassette. In some embodiments, the expression cassette comprises an MCS, which can be used for inserting a gene of interest that encodes the POI for recombinant production. In some embodiments, the expression cassette on the vectors provided herein can comprise a nucleotide sequence encoding the POI (a POI-encoding sequence).

As used interchangeably herein and understood in the art, a “polypeptide” or “peptide” refers to polymers of amino acids of any length joined together by peptide bonds. It can include unnatural or modified amino acids or be interrupted by non-amino acids. A “protein” contains one or more polypeptides. In globular proteins such as enzymes, the polypeptide chain of amino acids becomes folded into a three-dimensional functional shape or tertiary structure, at least partly via disulfide (S—S) bonds with other amino acids in the same polypeptide. Other interactions such as hydrogen bonds, ionic bonds, covalent bonds, and hydrophobic interactions also contribute to the tertiary structure. In some proteins, such as antibody molecules and hemoglobin, several polypeptides bond together to form a quaternary structure. A polypeptide, peptide, or protein can also be modified with, for example, glycosylation, lipidation, acetylation, phosphorylation, or any other manipulation or modification.

Vectors provided herein include single-gene vectors, double-gene vectors, and multi-gene vectors. In some embodiments, the POI that can be expressed by the vectors provided herein comprises one or more copies of the same polypeptide. In some embodiments, provided herein are single-gene vectors comprising one nucleotide sequence encoding a polypeptide. In some embodiments, the POI that can be expressed by the vectors provided herein comprises two or more different polypeptides. In some embodiments, the two or more different polypeptides are each encoded by separate nucleotide sequences on separate vectors. The separate vectors can be co-introduced to the same host cell for recombinant production. In some embodiments, the different polypeptides that form the POI are each encoded by a separate nucleotide sequence on the same vector. In some embodiments, provided herein are double-gene vectors comprising two nucleotide sequences, each encoding a polypeptide. In some embodiments, provided herein are multi-gene vectors comprising multiple nucleotide sequences, each encoding a polypeptide of interest.

In some embodiments, vectors provided herein are double-gene vectors or multi-gene vectors comprising two or more nucleotide sequences encoding two or more polypeptides that are part of the same POI. In some embodiments, vectors provided herein are double-gene vectors or multi-gene vectors comprising two or more nucleotide sequences encoding two or more polypeptides that form more than one POI. The two or more nucleotide sequences encoding polypeptides of interest can be placed in one or more expression cassette. As such, in some embodiments, vectors provided herein can have a single bicistronic, tricistronic, or multicistronic expression cassette, wherein all encoding nucleotide sequences are operationally linked to one common expression control sequence. In some embodiments, vectors provided herein can have two or more expression cassettes, wherein the encoding nucleotide sequences are placed under control of the expression control sequences in different expression cassettes.

For example, in some embodiments, vectors provided herein comprise an expression cassette that comprises a first nucleotide sequence encoding a first polypeptide and a second nucleotide sequence encoding second polypeptide. The nucleotide sequences can be linked by a separating element. In some embodiments, vectors provided herein comprise a first expression cassette comprising the first nucleotide sequence and a second expression cassette comprising the second nucleotide sequence. In some embodiments, vectors provided herein comprise a bicistronic expression cassette comprising both the first and the second nucleotide sequences. The nucleotide sequences can be linked by separating elements.

The separating element contained in the vectors disclosed herein can be, for example, an IRES or 2A element. In some embodiments, a vector provided herein comprises a nucleotide encoding a 2A self-cleaving peptide. In some embodiments, the GS-encoding sequence and the POI-encoding sequence are linked by a 2A-encoding sequence. In some embodiments, the POI-encoding sequences are linked by a 2A-encoding sequence. Illustrative 2A self-cleaving peptides include P2A, E2A, F2A, and T2A. In some embodiments, a vector provided herein comprises an IRES. In some embodiments, the GS-encoding sequence and the POI-encoding sequence are linked by an IRES. In some embodiments, the POI-encoding sequences are linked by an IRES.

For example, in some embodiments, provided herein are expression vectors for recombinant production of an antibody having a light chain and a heavy chain, wherein the vectors comprise a first nucleotide sequence encoding the light chain and a second nucleotide sequence encoding the heavy chain. In some embodiments, the first and second nucleotide sequences are placed in the same expression cassette, separated by a 2A encoding sequence or an IRES. In some embodiments, the first and second nucleotide sequences are placed in a first and a second expression cassette, respectively.

The POIs that can be expressed by the vectors disclosed herein are described further in sections below. For example, the POI can be selected from the group consisting of an antibody, an enzyme, a soluble protein, a secreted protein, a membrane protein, and a fusion protein. In some embodiments, the POI is an antibody selected from the group consisting of an IgA antibody, an IgM antibody, an IgG1 antibody, an IgG2 antibody, an IgG3 antibody, an IgG4 antibody, a Fab, a Fab′, a F(ab′)2, a Fv, a scFv, a (scFv)2, a single domain antibody (sdAb), a single chain antibody (scAb), and a heavy chain antibody (HCAb). In some embodiments, the POI is an antibody selected from the group consisting of a monoclonal antibody, a bispecific antibody, a multi-specific antibody, a bivalent antibody, and a multivalent antibody.

In some embodiments, vectors provided herein are suitable for genomic integration. In some embodiments, vectors provided herein are suitable for recombinant protein production. In some embodiments, provided herein are expression vectors comprising a GS-encoding sequence and an expression cassette. A wide variety of expression vectors can be employed. Examples of vectors are plasmid, autonomously replicating sequences, and transposable elements. Exemplary vectors also include, without limitation, plasmids, phages phagemids, cosmids, fosmids, artificial chromosomes such as yeast artificial chromosome (YAC), bacterial artificial chromosome (BAC), P1-derived artificial chromosome (PAC), mammalian artificial chromosome (MAC), bacteriophages such as lambda phage or M13 phage, and animal viruses.

Examples of categories of animal viruses useful as vectors include, without limitation, retrovirus (including lentivirus), adenovirus, adeno-associated virus, cytomegalovirus, herpesvirus (e.g., herpes simplex virus), poxvirus, baculovirus, bovine papilloma virus, papillomavirus, and papovavirus (e.g., SV40). In some embodiments, expression vectors provided herein are adeno-associated virus (AAV) vectors, lentivirus vectors, retrovirus vectors, replication competent adenovirus vectors, replication deficient adenovirus vectors, a herpes virus vector, baculovirus vectors. In some embodiments, the vector is an adenovirus vector. In some embodiments, the vector is a retroviral vector. In some embodiments, the vector is an adeno-associated viral vector. Examples of expression vectors are pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. Exemplary transposon systems such as Sleeping Beauty and PiggyBac can also be used. (Ivics et al., Cell, 91 (4): 501-510 (1997); Cadiñanos et al., (2007) Nucleic Acids Research. 35 (12): e87).

In some embodiments, the vector is an episomal vector or a vector that is maintained extrachromosomally. As used herein, the term “episomal” refers to a vector that can replicate without integration into host's chromosomal DNA and without gradual loss from a dividing host cell also meaning that said vector replicates extrachromosomally or episomally.

In some embodiments, vector provided herein are engineered to harbor the sequence coding for the origin of DNA replication or “ori” from a lymphotrophic herpes virus or a gamma herpesvirus, an adenovirus, SV40, a bovine papilloma virus, or a yeast, specifically a replication origin of a lymphotrophic herpes virus or a gamma herpesvirus corresponding to oriP of EBV. In some embodiments, the lymphotrophic herpes virus can be Epstein Barr virus (EBV), Kaposi's sarcoma herpes virus (KSHV), Herpes virus saimiri (HS), or Marek's disease virus (MDV). Epstein Barr virus (EBV) and Kaposi's sarcoma herpes virus (KSHV) are also examples of a gamma herpesvirus. Typically, the host cell comprises the viral replication transactivator protein that activates the replication.

“Expression control sequences, control elements, or regulatory sequences present in an expression vector are those non-translated regions of the vector-origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements can vary in their strength and specificity. Depending on the vector system and host utilized, any number of suitable transcription and translation elements, including ubiquitous promoters and inducible promoters can be used.

Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

In some embodiments, vectors provided herein comprise a promoter operatively linked to the GS-encoding sequence. As understood in the art, a promoter refers to a nucleotide sequence that defines where transcription of a gene by RNA polymerase begins. Promoter sequences are typically located directly upstream or at the 5′ end of the transcription initiation site. DNA regions are operatively linked when they are functionally related to each other. Structures in a nucleotide sequence that are linked by operative ability are capable of, or characterized by, accomplishing a desired operation. It is recognized by one of ordinary skill in the art that it is not necessary for elements or structures in a nucleic acid sequence to be in a tandem or adjacent order to be operatively linked. For example, a promoter is operatively linked to a coding sequence if it controls the transcription of the sequence; or a ribosome binding site is operatively linked to a coding sequence if it is positioned to permit translation.

In some embodiments, provided herein are expression vectors comprising a GS-encoding sequence and an expression cassette, wherein the GS-encoding sequence is operatively connected to a promoter. The promoter can be any promoter described herein or otherwise known in the art. In some embodiments, the promoter is a CMV promoter. In some embodiments, the promoter is a SV40 promoter.

As described above, the vector described herein can be used for recombinant production of a POI. In some embodiments, the vectors provided herein comprise a GS-encoding sequence and an expression cassette comprising a POI-encoding sequence. The expression cassette comprises a promoter operatively linked to the POI-encoding sequence. The promoter can be any promoter disclosed herein or otherwise known in the art. In some embodiments, the promoter is the same as the promoter operatively linked to the GS-encoding sequence. In some embodiments, the promoter is different from the promoter operatively linked to the GS-encoding sequence.

In some embodiments, the expression cassette comprises two or more nucleotide sequences encoding two or more polypeptides, wherein the two or more nucleotide sequences are operatively linked to the same promoter. In some embodiments, vectors provided herein comprise two or more expression cassettes, each comprising a promoter operatively linked to a nucleotide sequence encoding a polypeptide. In some embodiments, promoters in different expression cassettes are the same promoter. In some embodiments, promoters in different expression cassettes are different. The promoters can be any promoter disclosed herein or otherwise known in the art.

The promoter can be a forward promoter or a reverse promoter. In some embodiments, the promoter is a mammalian promoter. In some embodiments, one or more promoters are native promoters. In some embodiments, one or more promoters are non-native promoters. In some embodiments, one or more promoters are non-mammalian promoters.

Illustrative ubiquitous promoters that can be used in present disclosure include, but are not limited to, a cytomegalovirus (CMV) promoter, a viral simian virus 40 (SV40) promoter (e.g., early or late), a Moloney murine leukemia virus (MoMLV) LTR promoter, a Rous sarcoma virus (RSV) LTR, a herpes simplex virus (HSV) (thymidine kinase) promoter, a spleen focus-forming virus (SFFV) promoter, a U1 promoter, a U6 promoter, a H1 promoter, a H5 promoter, a P7.5 promoter, a P11 promoter, a T7 promoter, a Sp6 promoter, an elongation factor 1-alpha (EF1α) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), a lac promoter, the human ROSA 26 locus (Irions et al., Nature Biotechnology 25, 1477-82 (2007)), an upstream activation sequence (UAS) promoter, a Ubiquitin C promoter (UBC), a phosphoglycerate kinase-1 (PGK) promoter, a cytomegalovirus enhancer/chicken β-actin (CAG) promoter, a tetracycline response element (TRE), an araC promoter, an araBAD promoter, a tryptophan (trp) promoter, a Ptac promoter, and a β-actin promoter. In some embodiments, a CMV promoter is used. In some embodiments, a SV40 promoter is used. In some embodiments, an EF1α promoter is used.

In some embodiments, the promoter drives the expression constitutively; that is, the promoter is a constitutive promoter. In some embodiments, the promoter is an inducible promoter. The inducible promoter is not limited, and can be any inducible promoter known in the art. In some embodiments, the expression of the inducible promoter is promoted by the presence of one or more environmental or chemical stimuli. For instance, in some embodiments, the inducible promoter drives expression in the presence of a chemical molecule such as tetracycline and derivatives thereof (such as, doxycycline), cumate and derivatives thereof; or environmental stimuli, such as heat or light.

In some embodiments, the inducible promoter is based on the tetracycline-controlled transcriptional activation system, the cumate repressor system, the lac repressor system, arabinose-regulated pBad promoter system, alcohol-regulated AlcA promoter system, steroid-regulated LexA promoter system, heat shock inducible Hsp70 or Hsp90 promoter system, or blue light inducible pR promoter system. Thus, in some embodiments, the inducible promoter comprises a nucleic acid sequence that binds to a tetracycline transactivator, such as a tetracycline response element. In some embodiments, the expression of the inducible promoter is turned on in the presence of tetracycline and derivatives thereof (Tet-On system), while in other embodiments, the expression of the inducible promoter is turned off in the presence of tetracycline and derivatives thereof (Tet-Off system). In some embodiments, the inducible promoter is based on the cumate repressor system. Thus, in some embodiments, the inducible promoter comprises a nucleic acid sequence that binds to a CymR repressor, such as a cumate operator sequence.

In some embodiments, the expression of the inducible promoter is driven by the dimerization of a transcription factor. In some embodiments, the transcription is bacterial EL222, which dimerizes in the presence of blue light to drive expression from Cl 20 promoter or a regulatory element thereof. In some embodiments, the inducible promoter comprises a nucleic acid sequence derived from the C120 promoter or regulatory element. Illustrative examples of inducible promoters/systems further include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

In some embodiments, provided herein are vectors suitable for recombinant protein expression in mammalian cells (e.g., CHO cells), comprising: a nucleotide sequence encoding an Alligator, green anole, or spotted gar GS disclosed herein operatively linked to a promoter, and an expression cassette comprising a nucleotide sequence encoding a POI operatively linked to a promoter. The promoters can be any promoter disclosed herein or otherwise known in the art. In some embodiments, the promoters are the same. In some embodiments, the promoters are different. In some embodiments, the POI is an antibody comprising a heavy chain and a light chain. Accordingly, in some embodiments, provided herein are vectors suitable for recombinant antibody expression in mammalian cells (e.g., CHO cells), comprising: a nucleotide sequence encoding an Alligator, green anole, or spotted gar GS disclosed herein operatively linked to a promoter, a first expression cassette comprising a nucleotide sequence encoding the light chain operatively linked to a promoter, and a second expression cassette comprising a nucleotide sequence encoding the heavy chain operatively linked to a promoter. In some embodiments, provided herein are vectors suitable for recombinant antibody expression in mammalian cells (e.g., CHO cells), comprising: a nucleotide sequence encoding an Alligator, green anole, or spotted gar GS disclosed herein operatively linked to a promoter, and an expression cassette comprising a promoter that is operatively linked to a first nucleotide sequence encoding the light chain and a second nucleotide sequence encoding the heavy light chain. The promoters can be any promoter disclosed herein or otherwise known in the art, such as CMV promoter and SV40 promoter. In some embodiments, the promoters are the same. In some embodiments, the promoters are different.

For expression in mammalian cells, the 3′-ends of most mammalian mRNAs are polyadenylated or are connected to multiple adenine that forms poly(A) tail. In some embodiments, vectors provided herein comprise one or more polyadenylation signals. When more than one polyadenylation signals are provided, the polyadenylation signal can be located at the 3′ end of each set of nucleotide sequences that encode for a polypeptide. Thus, in one example, the vectors provided herein comprise one polyadenylation signal at the 3′ end of each of the at least one POI-encoding sequence, and one polyadenylation signal at the 3′ end of the GS-encoding sequence. In some embodiments, the polyadenylation signal can be provided at the 3′ end of each expression cassette. For illustrative purposes, vectors provided herein can comprise nucleotide sequences encoding a heavy chain and a light chain of an antibody, wherein the heavy chain-encoding sequence and the light chain-encoding sequence are placed in the same expression cassette. In some embodiments, such vectors can comprise one polyadenylation signal at 3′ end of the expression cassette, and one polyadenylation signal at 3′ end of the GS-encoding sequence. In some embodiments, such vectors can comprise one polyadenylation signal at 3′ end of the heavy chain-encoding sequence, one polyadenylation signal at 3′ end of the light chain-encoding sequence, and one polyadenylation signal at 3′ end of the GS-encoding sequence.

### 5.3 Host Cells

Provided herein are host cells comprising a vector described herein. The vector can be any vector disclosed herein. In some embodiments, provided herein are host cells comprising a vector that has a GS-encoding sequence disclosed herein, as well as an expression cassette that has either a MCS for inserting a nucleotide sequence encoding a POI (a POI-encoding sequence), or a POI-encoding sequence. In some embodiments, provided herein are host cells comprising a POI-encoding sequence inserted at a transcriptionally active locus generated using the selectable markers described herein or methods described herein. In some embodiments, provided herein are stable cell lines comprising a vector described herein.

In some embodiments, host cells provided herein can grow in a glutamine-free medium. In some embodiments, host cells provided herein can grow in the presence of a GS inhibitor. The GS inhibitor can be any GS inhibitor disclosed herein or otherwise known in the art, including MSX and derivatives thereof, phosphorus containing analogues of glutamic acid, and bisphosphonates. In some embodiments, the GS inhibitor is MSX or a derivative thereof. In some embodiments, the GS inhibitor is a phosphorus containing analogues of glutamic acid. In some embodiments, the GS inhibitor is a bisphosphonate. The GS inhibitors can be supplemented at different concentrations to generate different levels of selection stringency.

In some embodiments, host cells provided herein can grow in a culture medium supplemented with MSX at about 1-10 μM, about 10-50 μM, about 50-100 μM, or about 100-300 μM. In some embodiments, host cells provided herein can grow in a culture medium supplemented with MSX at about 1 μM, about 3 μM, about 5 μM, about 10 μM, about 25 μM, about 50 μM, about 75 μM, about 100 μM, about 150 μM, about 200 μM, about 250 μM, or about 300 μM. In some embodiments, host cells provided herein can grow in a culture medium supplemented with MSX at about 5 μM. In some embodiments, host cells provided herein can grow in a culture medium supplemented with MSX at about 10 μM. In some embodiments, host cells provided herein can grow in a culture medium supplemented with MSX at about 50 μM. In some embodiments, host cells provided herein can grow in a culture medium supplemented with MSX at about 100 μM.

Host cells provided herein can comprise one vector. In some embodiments, host cells can comprise one vector comprising a GS-encoding sequence disclosed herein and an expression cassette. As described above, one expression cassette can comprise one or more nucleotide sequence(s) encoding one or more polypeptide(s) of interest. In some embodiments, host cells provided herein can comprise one vector comprising a GS-encoding sequence disclosed herein and two or more expression cassettes. A host cell can comprise multiple copies of the same vector. In some embodiments, host cells can comprise two different vectors, each comprising a GS-encoding sequence disclosed herein and at least one expression cassette. In some embodiments, host cells can comprise multiple different vectors, each comprising a GS-encoding sequence disclosed herein and at least one expression cassette.

In some embodiments, the host cells provided herein can have wild-type endogenous GS. In some embodiments, the endogenous GS of the host cell has mutation that results in reduced activity. In some embodiments, the endogenous GS of the host cell is inactivated. In some embodiments, the endogenous GS of the host cell is knocked out. Cells wherein the GS genes are knocked out lose their endogenous GS activity and cannot grow in glutamine-free environment without insertion or incorporation of an exogenous GS gene from, for example, the vectors described herein.

The host cells provided herein can be eukaryotic cell lines, for instance, a yeast cell line (e.g., a Saccharomyces cerevisiae or a Yarrowia lipolytica cell line), a fungal cell line (e.g., an Aspergillus niger cell line), an insect cell line (e.g., a Spodoptera fugiperda cell line, such as Sf9), or a mammalian cell line. Examples of suitable mammalian host cell lines include, but are not limited to, COS-7 (monkey kidney-derived), L-929 (murine fibroblast-derived), C127 (murine mammary tumor-derived), NSO (nonsecreting murine myeloma-derived), SP2/0 (murine myeloma-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines (e.g., HEK293-F, HEK293-H, HEK293-T), PERC.6 (human embryonic retinoblasts-derived), HROC277 (Human Colorectal Adenocarcinoma cell-derived), VERO (African green monkey kidney-derived), MDCK (Canine kidney-derived), W138 (Human lung fibroblasts-derived), V79 (Chinese Hamster lung-derived), BHK (Baby Hamster Kidney fibroblasts-derived), and variants thereof.

In some embodiments, host cells provided herein are COS-7 cells. In some embodiments, host cells provided herein are L-929 cells. In some embodiments, host cells provided herein are C127 cells. In some embodiments, host cells provided herein are NSO cells. In some embodiments, host cells provided herein are SP2/0 cells. In some embodiments, host cells provided herein are 3T3 cells. In some embodiments, host cells provided herein are CHO cells. In some embodiments, host cells provided herein are HeLa cells. In some embodiments, host cells provided herein are BHK cells. In some embodiments, host cells provided herein are HEK-293 cells. In some embodiments, host cells provided herein are PERC.6 cells. In some embodiments, host cells provided herein are HROC277 cells. In some embodiments, host cells provided herein are VERO cells. In some embodiments, host cells provided herein are MDCK cells. In some embodiments, host cells provided herein are WI38 cells. In some embodiments, host cells provided herein are V79 cells. In some embodiments, host cells provided herein are BHK cells.

In some embodiments, the host cells provided herein are suitable for post-translational modifications (“PTMs”), such as glycosylation, phosphorylation, disulfide bonds, in the POI.

In some embodiments, the cell line is a stable cell line. As understood in the art, a stable cell line refers to a cell clone comprising a vector described herein that can sustainably express a POI. In some embodiments, the cell is transiently introduced with any one or more of the vectors disclosed herein. In some embodiments, provided herein are host cells for expression of a POI, wherein the cell comprises a vector disclosed herein comprising a GS-encoding sequence and an expression cassette comprising a POI-encoding sequence. In some embodiments, the vector is transiently introduced into the host cell, and not integrated into the genome of the cell. In some embodiments, the coding sequences of the vector are stably integrated into the genome of the cell.

In some embodiments, the host cells are eukaryotic cells. In some embodiments, the host cells are mammalian cells. In some embodiments, the host cells are Chinese Hamster Ovary (CHO) cells. More than half of the therapeutic proteins approved and currently marketed are produced using CHO cells, mainly due to CHO cells' unique characteristics, such as human-like post-translational modification of the product and their amenability to bioprocess development and large-scale manufacturing. In some embodiments, provided herein are CHO cells comprising a vector provided herein. The vector can be any vector described herein.

In some embodiments, host cells included in the methods described above disclosed herein are CHO cells. In some embodiments, the CHO cells have a wild-type endogenous GS. Exemplary CHO cell lines with wild-type GS can be, for example, CHO-S, CHO-K1, CHOK1SV, CHOZN K1, or FreeStyle CHO-S. In some embodiments, the CHO cells having a wild-type GS can carry a mutation in gene(s) other than GS that reduces or eliminates the enzymatic activity of a protein involved in, for example, glycosylation or metabolic pathway. The mutation can be a naturally occurring mutation or genetically engineered mutation. Exemplary CHO cell lines carrying an impaired or inactivated glycosylation enzyme (e.g., fucosyltransferase 8) include CHO FUT8 KO. Exemplary CHO cell lines carrying an impaired or inactivated enzyme in metabolic pathway (e.g., dihydrofolate reductase, DHFR) include CHO-DG44, CHO-DUXB11, and CHO-DUKX. In some embodiments, the CHO cells having a wild-type GS can have enhanced enzymatic activities in gene(s) other than GS, these gene product(s) are involved in cell growth/viability, metabolism, protein modification. In some embodiments, the CHO cells carry amplification of a gene encoding an anti-apoptotic protein. The gene amplification can result from a naturally occurring mutation or a genetically engineered alteration. In some embodiments, the CHO cells carry an exogenous gene encoding an anti-apoptotic protein.

In some embodiments, host cells provided herein are CHO-S cells. In some embodiments, host cells provided herein are CHO-K1 cells. In some embodiments, host cells provided herein are CHOK1SV cells. In some embodiments, host cells provided herein are CHOZNK1 cells. In some embodiments, host cells provided herein are FreeStyle CHO-S cells. In some embodiments, host cells provided herein are CHO-DG44 cells. In some embodiments, host cells provided herein are CHO-DUXB11 cells. In some embodiments, host cells provided herein are CHO-DUKX cells.

In some embodiments, the endogenous GS of the CHO cells provided herein have reduced activity compared to a wild-type hamster GS. In some embodiments, the endogenous GS of the CHO cells provided herein is inactivated. In some embodiments, the endogenous GS of the CHO cells provided herein is knocked out. Exemplary CHO cell lines with endogenous GS knocked out can be, for example, CHOK1SV GS-KO, CHOZN GS−/−, or CHOZN GS KO. In some embodiments, host cells provided herein are CHOK1SV GS-KO cells. In some embodiments, host cells provided herein are CHOZN GS−/− cells. In some embodiments, host cells provided herein are CHOZN GS KO cells.

These CHO cells described herein are commercially available from vendors such as Lonza, ECACC, Sigma-Aldrich/Merck, Fisher, and Horizon. Most CHO lines used for recombinant protein production were initially derived from CHO K1 (Kao & Puck, 1968), and therefore are very similar genetically.

In some embodiments, CHO cells provided herein can grow in a glutamine-free medium. In some embodiments, CHO cells provided herein can grow in the presence of a GS inhibitor. In some embodiments, CHO cells provided herein can grow in a glutamine-free medium in the presence of a GS inhibitor. In some embodiments, CHO cells provided herein can be selected for their ability to grow in a glutamine-free medium. In some embodiments, CHO cells provided herein can be selected for their ability to grow in the presence of a GS inhibitor. In some embodiments, CHO cells provided herein can be selected for their ability to grow in a glutamine-free medium in the presence of a GS inhibitor. The GS inhibitor can be any GS inhibitor disclosed herein or otherwise known in the art, including MSX and derivatives thereof, phosphorus containing analogues of glutamic acid, and bisphosphonates. In some embodiments, CHO cells provided here can grow in a glutamine-free medium supplemented with MSX. In some embodiments, CHO cells provided here can be selected for their ability to grow in a glutamine-free medium supplemented with MSX. The GS inhibitors can be supplemented at different concentrations for creating different levels of selection stringency. In some embodiments, the MSX is supplemented at about 1-10 μM, about 10-50 μM, about 50-100 μM, or about 100-300 μM. In some embodiments, the MSX is supplemented at about 1 μM, about 3 μM, about 5 μM, about 10 μM, about 25 μM, about 50 μM, about 75 μM, about 100 μM, about 150 μM, about 200 μM, about 250 μM, or about 300 μM. In some embodiments, the MSX is supplemented at about 50 μM.

### 5.4 Protein of Interest (POI)

Provided herein are uses of GS that are derived from Alligator, green anole, or spotted gar as selectable markers in recombinant production of POI, or in some embodiments, recombinant production of an mRNA of interest. The POI can be any protein for which expression is desired. In some embodiments, the POI is an antibody, an enzyme, a soluble protein, a secreted protein, a membrane protein, or a fusion protein. In some embodiments, antibody can be a therapeutic protein. In some embodiments, a POI is an antibody. In some embodiments, the POI is an enzyme. In some embodiments, the enzyme can be used for enzyme replacement therapy. In some embodiments, the POI is a soluble protein. In some embodiments, the POI is a secreted protein. In some embodiments, the POI is a membrane protein. In some embodiments, the POI is a fusion protein.

In some embodiments, the expression of the POI may cause cell toxicity when expressed in a reference expression system. In some embodiments, the POI is a protein with low yield expression in traditional expression systems. In some embodiments, the expression or quality of the protein is significantly improved by expression according to the disclosed methods, e.g., using an Alligator, green anole, or spotted gar GS as selectable marker.

In some embodiments, the POI is a human protein. In some embodiments, the POI is a mammalian protein.

In some embodiments, the POI is a monomer, namely, consists of a single polypeptide. In some embodiments, the POI comprises two or more copies the same polypeptide. In some embodiments, the POI is a multimer and comprises at least two different polypeptides. In some embodiments, the POI comprises unnatural amino acids. In some embodiments, POIs provided herein include post-translational modifications (“PTMs”), such as glycosylation, phosphorylation, ubiquitination, nitrosylation, methylation, acetylation, lipidation, etc.

In some embodiments, the POI is an antibody. Exemplary antibodies that can be produced by the compositions and methods disclosed herein include intact monoclonal antibodies, single-domain antibodies (sdAbs; e.g., camelid antibodies, alpaca antibodies), single-chain Fv (scFv) antibodies, heavy chain antibodies (HCAbs), light chain antibodies (LCAbs), multispecific antibodies, bispecific antibodies, monospecific antibodies, monovalent antibodies, and any other modified immunoglobulin molecule comprising an antigen-binding site (e.g., dual variable domain immunoglobulin molecules) as long as the antibodies exhibit the desired biological activity. Antibodies also include, but are not limited to, mouse antibodies, camel antibodies, chimeric antibodies, humanized antibodies, and human antibodies. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. Unless expressly indicated otherwise, the term “antibody” as used herein include “antigen-binding fragment” of intact antibodies. The term “antigen-binding fragment” as used herein refers to a portion or fragment of an intact antibody that is the antigenic determining variable region of an intact antibody. Examples of antigen-binding fragments include, but are not limited to, Fab, Fab′, F(ab′)2, Fv, linear antibodies, single chain antibody molecules (e.g., scFv), heavy chain antibodies (HCAbs), light chain antibodies (LCAbs), disulfide-linked scFv (dsscFv), diabodies, tribodies, tetrabodies, minibodies, dual variable domain antibodies (DVD), single variable domain antibodies (sdAbs; e.g., camelid antibodies, alpaca antibodies), and single variable domain of heavy chain antibodies (VHH), and bispecific or multispecific antibodies formed from antibody fragments.

Some antibodies comprise at least one heavy chain and one light chain. The term “heavy chain” when used in reference to an antibody refers to a polypeptide chain of about 50-70 kDa, wherein the amino-terminal portion includes a variable region of about 120 to 130 or more amino acids and a carboxy-terminal portion that includes a constant region. The constant region can be one of five distinct types, referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while μ and ε contain approximately 550 amino acids. When combined with a light chain, these distinct types of heavy chains give rise to five well known classes of antibodies, IgA, IgD, IgE, IgG and IgM, respectively, including four subclasses of IgG, namely IgG1, IgG2, IgG3 and IgG4. A heavy chain can be a human heavy chain. The term “light chain” when used in reference to an antibody refers to a polypeptide chain of about 25 kDa, wherein the amino-terminal portion includes a variable region of about 100 to about 110 or more amino acids and a carboxy-terminal portion that includes a constant region. The approximate length of a light chain is 211 to 217 amino acids. There are two distinct types, referred to as kappa (κ) of lambda (λ) based on the amino acid sequence of the constant domains. Light chain amino acid sequences are well known in the art. A light chain can be a human light chain.

In some embodiments, the POI is a therapeutic protein. In some embodiments, the POI can be useful in the production of clinical testing kits or other diagnostic assays.

In some embodiments, the present compositions and methods are used to produce therapeutic proteins. In some embodiments, the POI is selected from the group consisting of Abarelix, Abatacept, Abciximab, Adalimumab, Aflibercept, Agalsidase beta, Albiglutide, Aldesleukin, Alefacept, Alemtuzumab, Alglucerase, Alglucosidase alfa, Alirocumab, Aliskiren, Alpha-1-proteinase inhibitor, Alteplase, Anakinra, Ancestim, Anistreplase, Anthrax immune globulin human, Antihemophilic Factor, Antithrombin Alfa, Antithrombin III human, Antithymocyte globulin, Anti-thymocyte Globulin (Equine), Anti-thymocyte Globulin (Rabbit), Aprotinin, Arcitumomab, Asfotase Alfa, Asparaginase, Asparaginase Erwinia chrysanthemi, Atezolizumab, Autologous cultured chondrocytes, Basiliximab, Becaplermin, Belatacept, Belimumab, Beractant, Bevacizumab, Bivalirudin, Blinatumomab, Botulinum Toxin Type A, Botulinum Toxin Type B, Brentuximab vedotin, Brodalumab, Buserelin, Cl Esterase Inhibitor (Human), Cl Esterase Inhibitor, Canakinumab, Canakinumab, Capromab, Certolizumab pegol, Cetuximab, Choriogonadotropin alfa, Chorionic Gonadotropin (Human), Chorionic Gonadotropin, Coagulation factor IX, Coagulation factor Vila, Coagulation factor X human, Coagulation Factor XIII A-Subunit, Collagenase, Conestat alfa, Corticotropin, Cosyntropin, Daclizumab, Daptomycin, Daratumumab, Darbepoetin alfa, Defibrotide, Denileukin diftitox, Denosumab, Desirudin, Dinutuximab, Domase alfa, Drotrecogin alfa, Dulaglutide, Eculizumab, Efalizumab, Efmoroctocog alfa, Elosulfase alfa, Elotuzumab, Enfuvirtide, Epoetin alfa, Epoetin zeta, Eptifibatide, Etanercept, Evolocumab, Exenatide, Factor IX Complex (Human), Fibrinogen Concentrate (Human), Fibrinolysin aka plasmin, Filgrastim, Filgrastim-sndz, Follitropin alpha, Follitropin beta, Galsulfase, Gastric intrinsic factor, Gemtuzumab ozogamicin, Glatiramer acetate, Glucagon recombinant, Glucarpidase, Golimumab, Gramicidin D, Hepatitis A Vaccine, Hepatitis B immune globulin, Human calcitonin, Human Clostridium tetani toxoid immune globulin, Human rabies virus immune globulin, Human Rho(D) immune globulin, Human Serum Albumin, Human Varicella-Zoster Immune Globulin, Hyaluronidase, Hyaluronidase, Ibritumomab, Ibritumomab tiuxetan, Idarucizumab, Idursulfase, Imiglucerase, Immune Globulin Human, Infliximab, Insulin aspart, Insulin Beef, Insulin Degludec, Insulin detemir, Insulin Glargine, Insulin glulisine, Insulin Lispro, Insulin Pork, Insulin Regular, Insulin Regular, Insulin porcine, Insulin isophane, Interferon Alfa-2a, Interferon alfa-2b, Interferon alfacon-1, Interferon alfa-n1, Interferon alfa-n9, Interferon beta-1a, Interferon beta-1b, Interferon gamma-1b, Intravenous Immunoglobulin, Ipilimumab, Ixekizumab, Laronidase, Lenograstim, Lepirudin, Leuprolide, Liraglutide, Lucinactant, Lutropin alfa, Lutropin alfa, Mecasermin, Menotropins, Mepolizumab, Epoetin beta, Metreleptin, Muromonab, Natalizumab, alpha interferon, Necitumumab, Nesiritide, Nivolumab, Obiltoxaximab, Obinutuzumab, Ocriplasmin, Ofatumumab, Omalizumab, Oprelvekin, OspA lipoprotein, Oxytocin, Palifermin, Palivizumab, Pancrelipase, Panitumumab, Pembrolizumab, Pertuzumab, Poractant alfa, Pramlintide, Preotact, Protein S human, Ramucirumab, Ranibizumab, Rasburicase, Raxibacumab, Reteplase, Rilonacept, Rituximab, Romiplostim, Sacrosidase, Salmon Calcitonin, Sargramostim, Satumomab Pendetide, Sebelipase alfa, Secretin, Secukinumab, Sermorelin, Serum albumin, Serum albumin iodonated, Siltuximab, Simoctocog Alfa, Sipuleucel-T, Somatotropin Recombinant, Somatropin recombinant, Streptokinase, Sulodexide, Susoctocog alfa, Taliglucerase alfa, Teduglutide, Teicoplanin, Tenecteplase, Teriparatide, Tesamorelin, Thrombomodulin alfa, Thymalfasin, Thyroglobulin, Thyrotropin Alfa, Thyrotropin Alfa, Tocilizumab, Tositumomab, Trastuzumab, Tuberculin Purified Protein Derivative, Turoctocog alfa, Urofollitropin, Urokinase, Ustekinumab, Vasopressin, Vedolizumab, and Velaglucerase alfa.

In some embodiments, the POI is a soluble protein, a secreted protein, or a membrane protein. In some embodiments, the POI is, without limitation, Dopamine receptor 1 (DRD1), Cystic fibrosis transmembrane conductance regulator (CFTR), Cl esterase inhibitor (Cl-Inh), IL2 inducible T cell kinase (ITK), or an NADase. In some embodiments, the NADase is SARM1. In some embodiments, the SARM1 is a deletion variant that represents the mature protein.

In some embodiments, the POI is a membrane protein. Illustrative membrane proteins include ion channels, gap junctions, ionotropic receptors, transporters, integral membrane proteins such as cell surface receptors (e.g., G-protein coupled receptors (GPCRs), tyrosine kinase receptors, integrins and the like), proteins that shuttle between the membrane and cytosol in response to signaling (e.g., Ras, Rac, Raf, Ga subunits, arresting, Src and other effector proteins), and the like. In some embodiments, the POI is a G protein-coupled receptor. In some embodiments, the POI is a seven-(pass)-transmembrane domain receptor, 7TM receptor, heptahelical receptor, serpentine receptor, or G protein-linked receptor (GPLR). In some embodiments, the POI is a Class A GPCR, Class B GPCR, Class C GPCR, Class D GPCR, Class E GPCR, or Class F GPCR. In some embodiments, the POI is a Class 1 GPCR, Class 2 GPCR, Class 3 GPCR, Class 4 GPCR, Class 5 GPCR, or Class 6 GPCR. In some embodiments, the POI is a Rhodopsin-like GPCR, a Secretin receptor family GPCR, a Metabotropic glutamate/pheromone GPCR, a Fungal mating pheromone receptor, a Cyclic AMP receptor, or a Frizzled/Smoothened GPCR.

A POI for expression using the present compositions and methods can also include proteins related to enzyme replacement, such as Agalsidase beta, Agalsidase alfa, Imiglucerase, Taligulcerase alfa, Velaglucerase alfa, Alglucerase, Sebelipase alpha, Laronidase, Idursulfase, Elosulfase alpha, Galsulfase, Alglucosidase alpha, Factor VIII, C3 inhibitor, Hurler and Hunter corrective factors. In some embodiments, a POI is a nucleosidase, an NAD+ nucleosidase, a hydrolase, a glycosylase, a glycosylase that hydrolyzes N-glycosyl compounds, an NAD+ glycohydrolase, an NADase, a DPNase, a DPN hydrolase, an NAD hydrolase, a diphosphopyridine nucleosidase, a nicotinamide adenine dinucleotide nucleosidase, an NAD glycohydrolase, an NAD nucleosidase, or a nicotinamide adenine dinucleotide glycohydrolase. In some embodiments, the POI is an enzyme that participates in nicotinate and nicotinamide metabolism and calcium signaling pathway. In some embodiments, the POI can be a secreted protein, e.g., Cl-Inh.

POIs for expression using the present compositions and methods can also be fusion proteins. In some embodiments, the POI are fusion proteins comprising an Fc region. In some embodiments, the POI is a therapeutic protein comprising an Fc-region. In some embodiments, the POI is Alefacept, Etanercept, Abatacept, Belatacept, Aflibercept, Rilonacept, Romiplostim, Antihemophilic Factor-Fc Fusion Protein, or Eftrenonacog alfa.

In some embodiments, provided herein are also proteins expressed by introduction of a vector of the disclosure into a mammalian cell (e.g., a CHO cell). In some embodiments, provided herein are also proteins produced by mammalian cells comprising vectors of the disclosure.

### 5.5 Expression Systems and Kits

Using the selectable markers provided herein allows efficient identification of cell clones with high productivity of POI as well as improved production of POI. As such, provided herein are also expression systems for in vitro production of a POI comprising the vectors provided herein and/or the cells provided herein. In some embodiments, the vectors, cells, and systems provided herein are used for the reliable production of POIs that are difficult to express. In some embodiments, the expression systems efficiently identify host cells with high productivity, and result in improved productivity of POI (e.g., higher expression rate, higher yield). For illustration, the expression systems provided herein, and POIs produced therefrom can exhibit one or more of the following improvements: reliable production; decreased need for expression optimization; suitability for therapeutic applications; low batch-to-batch variation; improved functional activity; and consistent activity.

In some embodiments, expression systems provided herein comprise a vector disclosed herein and a host cell. The vector can be any vector disclosed herein. As provided, vectors in the expression systems provided herein can comprise a nucleotide sequence encoding an Alligator GS, a green anole GS, or a spotted gar GS. In some embodiments, the vectors are suitable for genomic integration. In some embodiments, the vectors are suitable for recombinant protein production and further comprise an expression cassette. The expression cassette can be empty (i.e., the POI-encoding sequence is yet-to-be inserted). The expression cassette can also comprise one or more POI-encoding sequences. In some embodiments, vectors provided herein can comprise two or more expression cassettes.

In some embodiments, the expression systems provided herein comprises one vector disclosed herein. In some embodiments, expression systems provided herein can comprise one vector comprising a GS-encoding sequence disclosed herein and an expression cassette. As described above, one expression cassette can comprise one or more nucleotide sequence(s) encoding one or more polypeptide(s) of interest. In some embodiments, expression systems provided herein can comprise one vector comprising a GS-encoding sequence disclosed herein and two or more expression cassettes. In some embodiments, expression systems provided herein can have two different vectors, each comprising a GS-encoding sequence disclosed herein and at least one expression cassette. In some embodiments, expression systems provided herein can have multiple different vectors, each comprising a GS-encoding sequence disclosed herein and at least one expression cassette.

Expression systems provided herein comprise a host cell described herein. In some embodiments, the host cells provided herein can have wild-type endogenous GS. In some embodiments, the endogenous GS of the host cell has mutation that results in reduced activity. In some embodiments, the endogenous GS of the host cell is inactivated. In some embodiments, the endogenous GS of the host cell is knocked out.

When the expression system provided herein is used for producing a recombinant POI, the vector is introduced into the host cell. In some embodiments, vector is stably introduced into the host cell. In some embodiments, vector is transiently introduced into the host cell. Accordingly, in some embodiments of the expression systems provided herein, the vector and the host cell are two separate components. In some embodiment of the expression systems provided herein, the vector is present within the host cell.

Any of the host cells disclosed herein or otherwise known in the art that can be used to recombinantly produce a POI can be used in the expression system disclosed herein. In some embodiments, the host cell is a eukaryote cell. In some embodiments, the host cell is a mammalian cell. Examples of suitable mammalian host cell lines include, but are not limited to, COS-7 (monkey kidney-derived), L-929 (murine fibroblast-derived), C127 (murine mammary tumor-derived), NSO (nonsecreting murine myeloma-derived), SP2/0 (murine myeloma-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines (e.g., HEK293-F, HEK293-H, HEK293-T), PERC.6 (human embryonic retinoblasts-derived), HROC277 (Human Colorectal Adenocarcinoma cell-derived), VERO (African green monkey kidney-derived), MDCK (Canine kidney-derived), W138 (Human lung fibroblasts-derived), V79 (Chinese Hamster lung-derived), BHK (Baby Hamster Kidney fibroblasts-derived), and variants thereof.

In some embodiments, host cells included in the expression systems disclosed herein are CHO cells. In some embodiments, the CHO cells have a wild-type endogenous GS. Exemplary CHO cell lines with wild-type GS can be, for example, CHO-S, CHO-K1, CHOK1SV, CHOZN K1, or FreeStyle CHO-S. In some embodiments, the CHO cells having a wild-type GS can carry a mutation in gene(s) other than GS that reduces or eliminates the enzymatic activity of a protein involved in, for example, glycosylation or metabolic pathway. The mutation can be a naturally occurring mutation or genetically engineered mutation. Exemplary CHO cell lines carrying an impaired or inactivated glycosylation enzyme (e.g., fucosyltransferase 8) include CHO FUT8 KO. Exemplary CHO cell lines carrying an impaired or inactivated enzyme in metabolic pathway (e.g., dihydrofolate reductase, DHFR) include CHO-DG44, CHO-DUXB11, and CHO-DUKX. In some embodiments, the CHO cells having a wild-type GS can have enhanced enzymatic activities in gene(s) other than GS, these gene product(s) are involved in cell growth/viability, metabolism, protein modification. In some embodiments, the CHO cells carry amplification of a gene encoding an anti-apoptotic protein. The gene amplification can result from a naturally occurring mutation or a genetically engineered alteration. In some embodiments, the CHO cells carry an exogenous gene encoding an anti-apoptotic protein.

In some embodiments, the endogenous GS of the CHO cells can have reduced activity compared to a wild-type hamster GS. In some embodiments, the endogenous GS of the CHO cells provided herein is inactivated. In some embodiments, the endogenous GS of the CHO cells provided herein is knocked out. Exemplary CHO cell lines with endogenous GS knocked out can be, for example, CHOK1 SV GS-KO, CHOZN GS−/−, or CHOZN GS KO.

In some embodiments, the expression systems provided herein further comprise a glutamine-free culture medium. Depending on the host cells included in the expression system, any suitable culture medium can be used. A variety of culture media are well known and commercially available in the art. For example, expression systems provided herein can comprise a CHO cell and culture medium suitable for CHO cells. Exemplary culture media for CHO cells can be, for example, Serum-Free Media (SFM), Protein-Free Media (PFM), and Chemically Defined Media (CDM) (Li et al., Front. Bioeng. Biotechnol., (2021) https://doi.org/10.3389/fbioe.2021.646363; Ritacco, et al., Biotechnology progress 34.6 (2018): 1407-1426).

Key components of the culture medium in the expression systems described herein include water, sources of carbon, nitrogen, and phosphate, certain amino acids, fatty acid, vitamin, trace elements, and salts. The water should be contaminant-free and endotoxin free. In some embodiments, Water For Injection (WFI) is used. Glucose can function as the primary energy and carbon source. Although CHO cells can maintain high viability in glucose-limiting media, due to the rapid growth and nutrient consumption rates of CHO cell in recombinant protein production media, the glucose level is typically controlled at high levels. Substitutes for glucose include galactose, fructose, mannose, and other hexoses. The choice of the carbon source can affect the glycosylation of the recombinant protein. For example, media containing a high concentration of mannose can inhibit intracellular α-mannosidase, thereby increasing the percentage of mannose glycosylation in the product, which can increase both antibody-dependent cell-mediated cytotoxicity (ADCC) and clearance rates of antibody in the human body.

Amino acids are also key components in cell culture media, and the maintenance of most amino acids at specific concentration ranges in the media can be crucial for CHO cell culture. Studies have shown that optimizing amino acid composition of cell culture media can improve growth profiles and titers, and can also achieve the desired product glycosylation patterns. In addition to enhancing titer and peak cell density, selected amino acids at certain concentrations can have protective effects for cells growing in bioreactors. Certain amino acids can also eliminate or alleviate some of the negative effects of ammonium and pCO2 accumulation, as well as high osmolality. Some amino acids also act as signal molecules, reducing the rate of apoptosis in mammalian cells. Essential amino acids include histidine, isoleucine, leucine, lysine, methionine, phenylalanine, threonine, tryptophan, and valine, which are supplied at high concentration in the culture media. Tryptophan, in particular, can be a limiting factor, and the supplementation of tryptophan has been shown to increase both titer and peak cell density. Despite that the nonessential amino acids can be synthesized by mammalian cells in culture, cell culture media still commonly contain most or all of these amino acids to support cell growth and protein production. In fact, most of the nonessential amino acids have a significant effect on cell culture processes. Further, some amino acid substitutions can help achieve improved solubility and stability. For example, tyrosine, the least soluble amino acid, can be replaced with phosphotyrosine disodium salt or tyrosine-containing dipeptides to improve solubility. Cysteine, one of the least stable amino acids, can be oxidized to cystine in neutral pH, which has low solubility. A highly soluble and stable cysteine derivative, S-Sulfocysteine, has been reported as a replacement cysteine source and anti-oxidant in CHO cell culture media.

Lipids are major components of biological membranes, and can also serve as energy sources and signaling molecules in mammalian cells. Generally, CHO cells can synthesize lipids on their own. However, lipid supplementation in serum-free medium has proven beneficial for cell viability and product glycosylation. Exogenous supplementation of phospholipids, such as phosphatidic acid and lysophosphatidic acid, has been demonstrated to stimulate CHO cell growth. As major constituents of phospholipids, choline and ethanolamine exhibit an enhancing effect on cell growth comparable to that of mixed lipid.

Vitamins serve as coenzymes, prosthetic groups, or cofactors in signal cascade as well as in enzyme inhibition and activation. Despite the trace amount needed, vitamins are essential components of cell culture media, especially in CDM. Vitamin addition has been shown to increase mAb volumetric yield up to 3-fold in CHO cell culture.

The effective concentrations of trace elements in cell culture media are typically very low and can even be below detection. However, their importance cannot be overlooked. For example, in CHO cell culture, concentrations of copper should be carefully optimized with respect to both culture performance and product quality. Also, iron is a necessary component in CDM; and zinc supplementation has been shown to provide not only a 1.2-fold enhancement in mAb production, but also reduced apoptosis. Other trace elements that can be included in the culture media disclosed herein include manganese, molybdenum, selenium, and vanadium, as well as germanium, rubidium, zirconium, cobalt, nickel, tin, and chromium for certain cells.

Salts play important chemical and biological roles in CHO cell culture media, including maintenance of cellular membrane potential, osmolality, and buffering. The bulk ions added to CHO media include sodium, potassium, magnesium, calcium, chloride, phosphate, (bi)carbonate, sulfate, and nitrate.

Growth factors, typically peptides, small proteins, and hormones, act as signal molecules influencing cell growth, proliferation, recovery and differentiation. In many early media, growth factors were supplied in the form of serum. In SFM, only a small number of specific growth factors are supplied, minimizing the overall complexity of the medium formulation. Widely-used growth factors include, for example, insulin and its analogs, and other autocrine growth factors, such as brain-derived neurotrophic factor (BDNF), fibroblast growth factor 8 (FGF8), growth regulated a protein (CXCL1), hepatocyte growth factor (HGF), hepatoma-derived growth factor (HDGF), leukemia inhibitory factor (LIF), macrophage colony stimulating factor 1 (CSF1), and vascular endothelial growth factor C (VEGFC). Alternatively, small molecule antioxidant chelator aurintricarboxylic acid (ATA) has been shown to promote CHO cell growth similar to insulin.

Polyamines are ubiquitous molecules in mammalian cells, which play key roles in multiple metabolic processes, including DNA synthesis and transcription, ribosome function, regulation of ion channels, and cell signaling. Although several polyamines are synthesized from ornithine by mammalian cells, supplementation of culture media with polyamines, such as putrescine, spermidine, and spermine, is essential to support and expedite CHO cell growth.

Non-nutritional components can be included in the culture media provided herein to provide a more stable physical or chemical environment for the cells. These components, which can include buffers, surfactants, and/or antifoam, can have a significant effect on cell growth and productivity. Exemplary buffers include a bicarbonate buffering system (C02/NaHCO₃), organic zwitterion buffers such as HEPES, or phosphate buffers. Exemplary surfactants include Pluronic F-68 and silicone-based antifoams such as Antifoam C (Sigma-Aldrich).

As such, the culture media provided herein can be supplemented to achieve higher titer, specific glycosylation pattern, cell density, etc. In some embodiments, the expression system provided herein can further include a supplement, such as an amino acid, a lipid, a trace element, a salt, or any other supplement disclosed herein or otherwise known in the art. (E.g, Ritacco et al., Biotechnology progress 34.6 (2018): 1407-26.) The supplement can be directly included in the culture medium or separated stored.

In some embodiments, the expression systems provided herein further comprise a GS inhibitor. The GS inhibitor can be any GS inhibitor disclosed herein or otherwise known in the art. The GS inhibitor can be MSX and derivatives thereof, phosphorus containing analogues of glutamic acid, or bisphosphonates. The GS inhibitors can be supplemented at different concentrations for creating different levels of selection stringency. In some embodiments, the expression systems provided herein further comprises MSX. In some embodiments, the expression systems provided herein comprises a culture medium and MSX. In some embodiments, the expression systems provided herein comprises a glutamine-free medium and MSX. In some embodiments, the medium and the MSX are two separate components of the expression system. In some embodiments, the medium is supplemented with the MSX. In some embodiments, the expression systems provided herein comprise a glutamine-free medium supplemented with MSX at about 1-10 μM, about 10-50 μM, about 50-100 μM, or about 100-300 μM. In some embodiments, the expression systems provided herein comprise a glutamine-free medium supplemented with MSX at about 1 μM, about 3 μM, about 5 μM, about 10 μM, about 25 μM, about 50 μM, about 75 μM, about 100 μM, about 150 μM, about 200 μM, about 250 μM, or about 300 μM.

In some embodiments, the expression systems provided herein further comprise a means for introducing the vector into the host cell. The means for introducing the vector into the cell can be any means known in the art. A polynucleotide or vector can be introduced into a host cell by a variety of methods, which are well known in the art and selected, in part, based on the host cell. For example, the vector can be introduced into a cell using chemical, physical, biological, or viral means. Methods of introducing a polynucleotide or a vector into a host cell include, but are not limited to, the use of calcium phosphate, dendrimers, cationic polymers, lipofection, liposomes, fugene, peptide dendrimers, electroporation, cell squeezing, sonoporation, optical transfection, protoplast fusion, impalefection, hydrodynamic delivery, injection, gene gun, magnetofection, particle bombardment, nucleofection, and viral transduction.

In some embodiments, expression systems provided herein can include means for inserting the vector into the genome of the host cell to produce stable cell lines. Means for genome integration include, for example, lentiviral transfection, baculovirus gene transfer into mammalian cells (BacMam), retroviral transfection, CRISPR/Cas9, and/or transposons. In some embodiments, expression systems provided herein can include means for transiently introducing the vector into a host cell. In some embodiments, means for transient transfection include the uses of viral vectors, helper lipids, e.g., PEI, Lipofectamine, or Fectamine 293.

The expression systems disclosed herein can for example be provided under the form of a kit. In some embodiments, the kit comprises one vial comprising the DNA vector, and another vial comprising the host cell. Accordingly, provided herein are kits for in vitro production of a POI comprising a DNA vector having a GS-encoding sequence and an expression cassette, and a host cell. In some embodiments, the host cell is a CHO cell. In some embodiments, kits provided herein further comprise a glutamine-free medium, MSX, or both. In some embodiments, the kit further comprises instructions for use.

### 5.6 Methods of Uses

Provided herein are also methods of uses of the selectable markers, vectors, cells, expression systems disclosed herein in, for example, the identification of genomic loci with high transcriptional activity, the identification of host cells with high expression of POI, and the in vitro recombinant protein production.

### 5.6.1 Methods of Screening

The selectable markers disclosed herein can be used for the identification of host cells with high expression of POI. The selectable markers disclosed herein can be used for the identification of genomic loci with high transcriptional activity. In some embodiments, the methods comprise (1) screening for a GS-expressing cell from a population of cells having a GS-encoding sequence disclosed herein integrated in random genomic loci thereof, and (2) identifying the genomic locus where the GS-encoding sequence is integrated in the GS-expressing cell, wherein the genomic locus represents a locus with high transcriptional activity. In some embodiments, the screening step comprises culturing the population of cells under conditions where only GS-expressing cells can grow. For example, the cells can be cultured in a glutamine-free medium. The cells can also be cultured in a medium supplemented with a GS inhibitor (e.g., MSX). The GS inhibitor can be supplemented at different concentrations to generate different levels of selection stringency. In some embodiments, the screening step comprises two or more rounds of screening. In some embodiments, the two or more rounds of screening involve different levels of stringency.

As such, in some embodiments, the methods provided herein comprise introducing a vector comprising a GS-encoding sequence described herein into a population of host cells, and culturing the population of host cells in a glutamine-free medium. The GS-encoding sequence is integrated into random loci of the genome of the host cells and only those having the GS-encoding sequence inserted at a genomic locus with high transcriptional activity can express GS at sufficient level to grow in glutamine-free medium. In some embodiments, the medium further comprise a GS inhibitor (e.g., an MSX). Any methods to introduce a nucleotide sequence into cells that are disclosed herein or otherwise known in the art can be used.

In some embodiments, the genomic loci can be located by sequencing. Any sequencing methods known in the art can be adopted. In some embodiments, next-generation sequencing is used. The genomic locus wherein the GS-encoding sequence is integrated in the GS-expressing cells can be identified using any methods known in the art. In some embodiments, the methods provided herein further comprise replacing the GS-encoding sequence with a POI-encoding sequence in the identified host cell. The replacement of the GS-encoding sequence with the POI-encoding sequence can be done with any methods known in the art. For example, in some embodiments, a recombinase can be used, such as Cre or flippase (Flp). In some embodiments, the replacement can be assisted by a DNA break(s) generated by enzymes such as a zinc finger nuclease, a transcription activator-like effector nuclease (TALEN), or a CRISPR-Cas system. The CRISPR-Cas system can be a CRISPR-Cas9 system (Cong et al. Science 2013. 339: 819-823).

The selectable markers and vectors disclosed herein can be used in the identification of host cells with high productivity of a POI. The methods comprise (1) introducing a vector disclosed herein having a GS-encoding sequence and a POI-encoding sequence into a population of host cells and (2) identifying the host cell that expresses the POI. Any methods of introducing the vector into the host cells that are disclosed herein or otherwise known in the art can be used in the methods disclosed herein. In some embodiments, the identification step comprises culturing the host cells under conditions where only GS-expressing cells can grow. For example, the cells can be cultured in a glutamine-free medium. The cells can also be cultured in a medium supplemented with a GS inhibitor (e.g., MSX). The GS inhibitor can be supplemented at different concentrations to generate different levels of selection stringency. In some embodiments, the screening step comprises two or more rounds of screening. In some embodiments, the two or more rounds of screening involve different levels of stringency. In some embodiments, provided herein are also methods of screening for a cell clone with high productivity of a POI, comprising culturing a population of cell clones transferred with a vector provided herein in a glutamine-free medium supplemented with a GS inhibitor, wherein the cell clone capable of growing in the culture medium is identified as the cell clone with high productivity of the POI.

In some embodiments, methods provided herein identify the host cells that express the POI at certain levels. For example, in some embodiments, methods provided herein identify the host cells that have a POI production titer ranging from 0.2-500 μg/mL at the early stage (e.g., in 96-well plates or culture tubes). In some embodiments, methods provided herein identify the host cells that have a POI production titer ranging from 0.05-20 g/L at the late stage (e.g., in flasks or bioreactors).

In some embodiments, the early-stage POI production can reach a titer of at least 1 μg/mL, at least 2 μg/mL, at least 5 μg/mL, at least 8 μg/mL, at least 10 μg/mL, at least 20 μg/mL, at least 50 μg/mL, at least 80 μg/mL, at least 100 μg/mL, at least 150 μg/mL, at least 200 μg/mL, at least 250 μg/mL, at least 300 μg/mL, at least 350 μg/mL, at least 400 μg/mL, at least 450 μg/mL, or at least 500 μg/mL. In some embodiments, the early-stage POI production can reach a titer of about 1 μg/mL, about 2 μg/mL, about 5 μg/mL, about 8 μg/mL, about 10 μg/mL, about 20 μg/mL, about 50 μg/mL, about 80 μg/mL, about 100 μg/mL, about 150 μg/mL, about 200 μg/mL, about 250 μg/mL, about 300 μg/mL, about 350 μg/mL, about 400 μg/mL, about 450 μg/mL, or about 500 μg/mL.

In some embodiments, the late-stage POI production can reach a titer of at least 0.05 g/L, at least 0.1 g/L, at least 0.2 g/L, at least 0.5 g/L, at least 0.8 g/L, at least 1 g/L, at least 2 g/L, at least 5 g/L, at least 8 g/L, at least 10 g/L, at least 12 g/L, at least 15 g/L, at least 18 g/L, or at least 20 g/L. In some embodiments, the late-stage POI production can reach a titer of about 0.05 g/L, about 0.1 g/L, about 0.2 g/L, about 0.5 g/L, about 0.8 g/L, about 1 g/L, about 2 g/L, about 5 g/L, about 8 g/L, about 10 g/L, about 12 g/L, about 15 g/L, about 18 g/L, or about 20 g/L.

In some embodiments, the early-stage POI production can reach a titer that is between 1-10 μg/mL, between 1-50 μg/mL, between 1-100 μg/mL, between 1-200 μg/mL, between 1-500 μg/mL, between 10-50 μg/mL, between 10-100 μg/mL, between 10-200 μg/mL, between 10-500 μg/mL, between 50-100 μg/mL, between 50-200 μg/mL, between 50-500 μg/mL, between 100-200 μg/mL, between 100-500 μg/mL, or between 200-500 μg/mL. In some embodiments, the late-stage POI production can reach a titer that is between 0.05-0.1 g/L, between 0.05-0.2 g/L, between 0.05-0.5 g/L, between 0.05-1 g/L, between 0.05-2 g/L, between 0.05-5 g/L, between 0.05-10 g/L, between 0.05-20 g/L, between 0.1-0.2 g/L, between 0.1-0.5 g/L, between 0.1-1 g/L, between 0.1-2 g/L, between 0.1-5 g/L, between 0.1-10 g/L, between 0.1-20 g/L, between 0.2-0.5 g/L, between 0.2-1 g/L, between 0.2-2 g/L, between 0.2-5 g/L, between 0.2-10 g/L, between 0.2-20 g/L, between 0.5-1 g/L, between 0.5-2 g/L, between 0.5-5 g/L, between 0.5-10 g/L, between 0.5-20 g/L, between 1-2 g/L, between 1-5 g/L, between 1-10 g/L, between 1-20 g/L, between 2-5 g/L, between 2-10 g/L, between 2-20 g/L, between 5-10 g/L, between 5-20 g/L, or between 10-20 g/L.

In some embodiments, methods provided herein comprise separating the population of host cells introduced with the vectors described herein into a number of pools, and measuring the POI expression of each pool to determine the pool of cells with the desired productivity.

The expression level or productivity of POI by host cells, or pools of host cells can be measured by any methods known in the art. Exemplary detection methods can involve immunohistochemistry, immunocytochemistry, flow cytometry (e.g., FACS), magnetic beads complexed with antibody molecules, ELISA assays, etc.

The host cells that can be used in the methods described above can be any cells that require glutamine for survival and growth. In some embodiments, the host cells can be eukaryotic cells, for instance, yeast cells (e.g., a Saccharomyces cerevisiae or a Yarrowia lipolytica cell line), fungal cells line (e.g., an Aspergillus niger cell line), insect cell lines (e.g., a Spodoptera fugiperda cell line, such as Sf9) or mammalian cells. Examples of suitable mammalian host cell lines include, but are not limited to, COS-7 (monkey kidney-derived), L-929 (murine fibroblast-derived), C127 (murine mammary tumor-derived), NSO (nonsecreting murine myeloma-derived), SP2/0 (murine myeloma-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines (e.g., HEK293-F, HEK293-H, HEK293-T), PERC.6 (human embryonic retinoblasts-derived), HROC277 (Human Colorectal Adenocarcinoma cell-derived), VERO (African green monkey kidney-derived), MDCK (Canine kidney-derived), W138 (Human lung fibroblasts-derived), V79 (Chinese Hamster lung-derived), BHK (Baby Hamster Kidney fibroblasts-derived), and variants thereof.

In some embodiments, host cells included in the methods described above disclosed herein are CHO cells. In some embodiments, the CHO cells have a wild-type endogenous GS. Exemplary CHO cell lines with wild-type GS can be, for example, CHO-S, CHO-K1, CHOK1SV, CHOZN K1, or FreeStyle CHO-S. In some embodiments, the CHO cells having a wild-type GS can carry a mutation in gene(s) other than GS that reduces or eliminates the enzymatic activity of a protein involved in, for example, glycosylation or metabolic pathway. The mutation can be a naturally occurring mutation or genetically engineered mutation. Exemplary CHO cell lines carrying an impaired or inactivated glycosylation enzyme (e.g., fucosyltransferase 8) include CHO FUT8 KO. Exemplary CHO cell lines carrying an impaired or inactivated enzyme in metabolic pathway (e.g., dihydrofolate reductase, DHFR) include CHO-DG44, CHO-DUXB11, and CHO-DUKX. In some embodiments, the CHO cells having a wild-type GS can have enhanced enzymatic activities in gene(s) other than GS, these gene product(s) are involved in cell growth/viability, metabolism, protein modification. In some embodiments, the CHO cells carry amplification of a gene encoding an anti-apoptotic protein. The gene amplification can result from a naturally occurring mutation or a genetically engineered alteration. In some embodiments, the CHO cells carry an exogenous gene encoding an anti-apoptotic protein.

In some embodiments, the endogenous GS of the CHO cells can have reduced activity compared to a wild-type hamster GS. In some embodiments, the endogenous GS of the CHO cells provided herein is inactivated. In some embodiments, the endogenous GS of the CHO cells provided herein is knocked out. Exemplary CHO cell lines with endogenous GS knocked out can be, for example, CHOK1 SV GS-KO, CHOZN GS−/−, or CHOZN GS KO.

To select for genomic loci with high transcriptional activity, or host cells that express high level of POI, a GS inhibitor can be used to generate a stringent selection condition. This is because high transcriptional activity/expression would be required to generate sufficient level of GS activity that allows the survival and growth of the host cell. Any GS inhibitor disclosed herein or otherwise known in the art, including MSX and derivatives thereof, phosphorus containing analogues of glutamic acid, and bisphosphonates, can be used in the methods disclosed herein. The GS inhibitors can be supplemented at different concentrations to generate different levels of stringency. In some embodiments, MSX is used. In some embodiments, the selection condition MSX at about 1-10 μM, about 10-50 μM, about 50-100 μM, or about 100-300 μM. In some embodiments, host cells provided herein can grow in a culture medium supplemented with MSX at about 1 μM, about 3 μM, about 5 μM, about 10 μM, about 25 μM, about 50 μM, about 75 μM, about 100 μM, about 150 μM, about 200 μM, about 250 μM, or about 300 μM.

For illustrative purposes, in some embodiments, provided herein are methods of identifying a CHO cell with high productivity of a POI, comprising (1) introducing a vector disclosed herein having an Alligator GS-encoding sequence and a POI-encoding sequence into a population of CHO cells and (2) identifying the cell that expresses the POI by culturing the population of CHO cells in a glutamine-free medium supplemented with about 50 μM MSX, and measuring the expression of POI by the surviving cells.

### 5.6.2 Methods of Production

The selectable markers, vectors, cells, and expression systems provided herein can be used in the in vitro production of POI. As such, provided herein are also uses of the selectable markers, vectors, cells, or expression systems disclosed herein for in vitro production of a POI. In some embodiments, provided herein are in vitro methods of producing a POI comprising culturing the host cells disclosed herein under conditions and for sufficient time to produce the POI. In some embodiments, the host cells comprise a vector disclosed herein that has a GS-encoding sequence disclosed herein and a POI-encoding sequence disclosed herein. In some embodiments, the host cells comprise a POI-encoding sequence inserted at a transcriptionally active genomic locus identified using the methods disclosed herein. The host cells can be any host cells disclosed herein that are suitable for recombinant production. In some embodiments, the host cells are CHO cells. In some embodiments, the host cells are CHO cells with wild-type endogenous GS. In some embodiments, the host cells are CHO cells of which the endogenous GS is knocked out.

The POI can be any POI disclosed herein or otherwise known in the art. In some embodiments, the POI is an antibody, an enzyme, a soluble protein, a secreted protein, a membrane protein, or a fusion protein.

In some embodiments, methods provided herein comprise identifying a host cell with high productivity of a POI using the selectable marker disclosed herein, and culturing the host cell under conditions for sufficient time to produce the POI.

Methods of culturing the host cells, such as the CHO cells for recombinant protein production are well known in the art. (Kim J Y et al., Appl Microbiol Biotechnol. 2012; 93(3):917-30; Ritacco F V et al., Biotechnol Prog. 2018; 34(6):1407-1426; Fischer S et al., Biotechnol Adv. 2015; 33(8):1878-96.) Using the selective markers provided herein, clones with high productivity can be identified. For example, in some embodiments, methods provided herein identify the host cells that have a POI production titer ranging from 0.2-500 μg/mL at early stage (e.g., in 96-well plates or culture tubes). In some embodiments, the in vitro methods provided herein can produce a POI at a titer of about 0.2-500 μg/mL. In some embodiments, the methods provided herein produce POI with a titer of at least 1 μg/mL, at least 2 μg/mL, at least 5 μg/mL, at least 8 μg/mL, at least 10 μg/mL, at least 20 μg/mL, at least 50 μg/mL, at least 80 μg/mL, at least 100 μg/mL, at least 150 μg/mL, at least 200 μg/mL, at least 250 μg/mL, at least 300 μg/mL, at least 350 μg/mL, at least 400 μg/mL, at least 450 μg/mL, or at least 500 μg/mL. In some embodiments, the methods provided herein produce POI with a titer of about 1 μg/mL, about 2 μg/mL, about 5 μg/mL, about 8 μg/mL, about 10 μg/mL, about 20 μg/mL, about 50 μg/mL, about 80 μg/mL, about 100 μg/mL, about 150 μg/mL, about 200 μg/mL, about 250 μg/mL, about 300 μg/mL, about 350 μg/mL, about 400 μg/mL, about 450 μg/mL, or about 500 μg/mL. In some embodiments, methods provided herein identify the host cells that have a POI production titer ranging from 0.05-20 g/L at late stage (e.g., in flasks or bioreactors). In some embodiments, the methods provided herein produce POI with a titer of at least 0.05 g/L, at least 0.1 g/L, at least 0.2 g/L, at least 0.5 g/L, at least 0.8 g/L, at least 1 g/L, at least 2 g/L, at least 5 g/L, at least 8 g/L, at least 10 g/L, at least 12 g/L, at least 15 g/L, at least 18 g/L, or at least 20 g/L.

In some embodiments, the methods provided herein produce POI with a titer of about 1 μg/mL, about 2 μg/mL, about 5 μg/mL, about 8 μg/mL, about 10 μg/mL, about 20 μg/mL, about 50 μg/mL, about 80 μg/mL, about 100 μg/mL, about 150 μg/mL, about 200 μg/mL, about 250 μg/mL, about 300 μg/mL, about 350 μg/mL, about 400 μg/mL, about 450 μg/mL, or about 500 μg/mL. In some embodiments, the methods provided herein produce POI with a titer of about 0.05 g/L, about 0.1 g/L, about 0.2 g/L, about 0.5 g/L, about 0.8 g/L, about 1 g/L, about 2 g/L, about 5 g/L, about 8 g/L, about 10 g/L, about 12 g/L, about 15 g/L, about 18 g/L, or about 20 g/L.

In some embodiments, the methods provided herein produce POI with a titer that is between 1-10 μg/mL, between 1-50 μg/mL, between 1-100 μg/mL, between 1-200 μg/mL, between 1-500 μg/mL, between 10-50 μg/mL, between 10-100 μg/mL, between 10-200 μg/mL, between 10-500 μg/mL, between 50-100 μg/mL, between 50-200 μg/mL, between 50-500 μg/mL, between 100-200 μg/mL, between 100-500 μg/mL, or between 200-500 μg/mL. In some embodiments, the methods provided herein produce POI with a titer that is between 0.05-0.1 g/L, between 0.05-0.2 g/L, between 0.05-0.5 g/L, between 0.05-1 g/L, between 0.05-2 g/L, between 0.05-5 g/L, between 0.05-10 g/L, between 0.05-20 g/L, between 0.1-0.2 g/L, between 0.1-0.5 g/L, between 0.1-1 g/L, between 0.1-2 g/L, between 0.1-5 g/L, between 0.1-10 g/L, between 0.1-20 g/L, between 0.2-0.5 g/L, between 0.2-1 g/L, between 0.2-2 g/L, between 0.2-5 g/L, between 0.2-10 g/L, between 0.2-20 g/L, between 0.5-1 g/L, between 0.5-2 g/L, between 0.5-5 g/L, between 0.5-10 g/L, between 0.5-20 g/L, between 1-2 g/L, between 1-5 g/L, between 1-10 g/L, between 1-20 g/L, between 2-5 g/L, between 2-10 g/L, between 2-20 g/L, between 5-10 g/L, between 5-20 g/L, or between 10-20 g/L.

The in vitro methods provided herein can produce a POI at a rate of about 1-800 μg/10⁶ cells/day. In some embodiments, the in vitro methods provided herein can produce a POI at at least 5 μg/10⁶ cells/day, at least 10 μg/10⁶ cells/day, at least 75 μg/10⁶ cells/day, at least 100 μg/10⁶ cells/day, at least 150 μg/10⁶ cells/day, at least 200 μg/10⁶ cells/day, at least 250 μg/10⁶ cells/day, at least 300 μg/10⁶ cells/day, at least 400 μg/10⁶ cells/day, or at least 500 μg/10⁶ cells/day. In some embodiments, the in vitro methods provided herein can produce a POI at about 5 μg/10⁶ cells/day, about 10 μg/10⁶ cells/day, about 75 μg/10⁶ cells/day, about 100 μg/10⁶ cells/day, about 150 μg/10⁶ cells/day, about 200 μg/10⁶ cells/day, about 250 μg/10⁶ cells/day, about 300 μg/10⁶ cells/day, about 400 μg/10⁶ cells/day, or about 500 μg/10⁶ cells/day. The expression level or productivity of POI by host cells, or pools of host cells can be measured by any methods known in the art. Exemplary detection methods can involve immunohistochemistry, immunocytochemistry, flow cytometry (e.g., FACS), magnetic beads complexed with antibody molecules, ELISA assays, etc.

In some embodiments, the methods provided herein further comprise separating the protein from other components in the culture. In some embodiments, the separating comprises extraction, continuous liquid-liquid extraction, pervaporation, membrane filtration, membrane separation, reverse osmosis, electrodialysis, free-flow-electrophoresis, affinity chromatography, immunoaffinity chromatography, high performance liquid chromatography, distillation, crystallization, centrifugation, extractive filtration, size exclusion chromatography, hydrophobic interaction chromatography, ion exchange chromatography, absorption chromatography, or ultrafiltration.

All papers, publications and patents cited in this specification are herein incorporated by reference as if each individual paper, publication or patent were specifically and individually indicated to be incorporated by reference and are incorporated herein by reference to disclose and describe the methods and/or materials in connection with which the publications are cited. However, mention of any reference, article, publication, patent, patent publication, and patent application cited herein is not, and should not be taken as an acknowledgment or any form of suggestion that they constitute valid prior art or form part of the common general knowledge in any country in the world.

Unless the context indicates otherwise, it is specifically intended that the various features described herein can be used in any combination.

Unless otherwise defined, all technical and scientific terms used herein have the same meaning as commonly understood by one of ordinary skill in the art to which this disclosure belongs.

### 5.7 Exemplary Embodiments

Embodiment 1: Use of a nucleotide sequence encoding a glutamine synthetase (GS) as a selectable marker, wherein the GS is an Alligator GS, a green anole GS, or a spotted gar GS.

Embodiment 2: The use of embodiment 1, wherein the GS is an Alligator GS derived from Alligatoridae.

Embodiment 3: The use of embodiment 2, wherein the GS has an amino acid sequence that is at least 90%, at least 92%, at least 95%, at least 98%, at least 99%, or 100% identical to SEQ ID NO:1.

Embodiment 4: The use of embodiment 2 or 3, wherein the GS has reduced activity compared to a wild-type Alligator GS.

Embodiment 5: The use of embodiment 1, wherein the GS has the amino acid sequence of SEQ ID NO:1.

Embodiment 6: The use of embodiment 1, wherein the GS is a green anole GS derived from Dactyloidae.

Embodiment 7: The use of embodiment 6, wherein the GS has an amino acid sequence that is at least 90%, at least 92%, at least 95%, at least 98%, at least 99%, or 100% identical to SEQ ID NO:2.

Embodiment 8: The use of embodiment 6 or 7, wherein the GS has reduced activity compared to a wild-type green anole GS.

Embodiment 9: The use of embodiment 1, wherein the GS has the amino acid sequence of SEQ ID NO:2.

Embodiment 10: The use of embodiment 1, wherein the GS is a spotted gar GS derived from Lepisosteidae.

Embodiment 11: The use of embodiment 10, wherein the GS has an amino acid sequence that is at least 90%, at least 92%, at least 95%, at least 98%, at least 99%, or 100% identical to SEQ ID NO:3.

Embodiment 12: The use of embodiment 10 or 11, wherein the GS has reduced activity compared to a wild-type spotted gar GS.

Embodiment 13: The use of embodiment 1, wherein the GS has the amino acid sequence of SEQ ID NO:3.

Embodiment 14: The use of embodiment 1, wherein the amino acid sequence of the GS comprises SEQ ID NO:4 with an amino acid substitution at a position selected from the group consisting of: H8, N10, G12, I13, Q15, M16, S19, E24, V33, G39, C49, C53, V54, E56, F68, S72, S80, V82, F85, E92, F98, F102, Q106, K107, P108, L113, H115, T116, K118, S125, Q127, H128, L139, D152, L160, R172, M176, K189, T191, Y194, K198, H199, I206, C209, R213, V220, K230, 1235, A236, T237, S240, T260, N265, H269, K271, A273, K276, S278, K279, R282, A287, F303, H304, K305, N308, N310, D311, D318, S320, T328, E332, A339, C341, F349, A350, I355, V356, N362, T364, Q367, F369, and Q370.

Embodiment 15: The use of embodiment 1, wherein the amino acid sequence of the GS comprises SEQ ID NO:4 with an amino acid substitution at a position selected from the group consisting of: N10, G12, S19, V33, C49, C53, V54, E56, S72, S80, V82, E92, F98, Q106, H128, D152, L160, R172, M176, T191, Y194, K198, H199, I206, R213, V220, K230, T237, S240, T260, K271, K305, D311, D318, S320, T328, A339, C341, F349, I355, Q367, and Q370.

Embodiment 16: The use of embodiment 15, wherein the amino acid sequence of the GS comprises SEQ ID NO:4 with about 3, about 5, about 10, about 15, about 20, about 25, about 30, about 35, or about 40 amino acid substitutions at positions selected from the group consisting of: N10, G12, S19, V33, C49, C53, V54, E56, S72, S80, V82, E92, F98, Q106, H128, D152, L160, R172, M176, T191, Y194, K198, H199, I206, R213, V220, K230, T237, S240, T260, K271, K305, D311, D318, S320, T328, A339, C341, F349, I355, Q367, and Q370.

Embodiment 17: Use of a nucleotide sequence encoding a GS as a selectable marker, wherein the GS comprises a catalytic domain from an Alligator GS, a green anole GS, or a spotted gar GS.

Embodiment 18: The use of embodiment 17, wherein the GS comprises a catalytic domain from an Alligator GS having an amino acid sequence that is at least 90%, at least 92%, at least 95%, at least 98%, at least 99%, or 100% identical to SEQ ID NO:1.

Embodiment 19: The use of embodiment 18, wherein the catalytic domain has an amino acid sequence that is at least 90%, at least 92%, at least 95%, at least 98%, at least 99%, or 100% identical to amino acids 110-359 of SEQ ID NO:1.

Embodiment 20: The use of embodiment 17, wherein the GS comprises a catalytic domain from a green anole GS having an amino acid sequence that is at least 90%, at least 92%, at least 95%, at least 98%, at least 99%, or 100% identical to SEQ ID NO:2.

Embodiment 21: The use of embodiment 20, wherein the catalytic domain has an amino acid sequence that is at least 90%, at least 92%, at least 95%, at least 98%, at least 99%, or 100% identical to amino acids 110-359 of SEQ ID NO:2.

Embodiment 22: The use of embodiment 17, wherein the GS comprises a catalytic domain from a spotted gar GS having an amino acid sequence that is at least 90%, at least 92%, at least 95%, at least 98%, at least 99%, or 100% identical to SEQ ID NO:3.

Embodiment 23: The use of embodiment 22, wherein the catalytic domain has an amino acid sequence that is at least 90%, at least 92%, at least 95%, at least 98%, at least 99%, or 100% identical to amino acids 113-362 of SEQ ID NO:3.

Embodiment 24: The use of any one of embodiments 1 to 23, wherein the GS-encoding nucleotide sequence is operatively linked to a mRNA destabilizing element.

Embodiment 25: The use of any one of embodiments 1 to 23, wherein the GS comprises a degron.

Embodiment 26: The use of embodiment 25, wherein the degron has an amino acid sequence selected from the group consisting of SEQ ID NOs:13-15.

Embodiment 27: The use of any one of embodiments 1 to 26, that is for identifying a genomic locus with high transcriptional activity.

Embodiment 28: The use of any one of embodiments 1 to 26, that is for identifying a host cell capable of producing a protein of interest (POI).

Embodiment 29: The use of any one of embodiments 1 to 26, that is for recombinant production of a POI.

Embodiment 30: The use of embodiment 29, that is for recombinant protein production in a mammalian cell.

Embodiment 31: The use of embodiment 30, wherein the mammalian cell is a Chinese Hamster Ovary (CHO) cell.

Embodiment 32: The use of any one of embodiments 28 to 31, wherein the POI is selected from the group consisting of an antibody, an enzyme, a soluble protein, a secreted protein, a membrane protein, and a fusion protein.

Embodiment 33: A deoxyribonucleic acid (DNA) vector suitable for recombinant protein production or genomic integration, comprising a nucleotide sequence encoding a GS (a GS-encoding sequence), wherein the GS is an Alligator GS, a green anole GS, or a spotted gar GS.

Embodiment 34: The vector of embodiment 33, wherein the GS is an Alligator GS derived from Alligatoridae.

Embodiment 35: The vector of embodiment 34, wherein the GS has an amino acid sequence that is at least 90%, at least 92%, at least 95%, at least 98%, at least 99%, or 100% identical to SEQ ID NO:1.

Embodiment 36: The vector of embodiment 34 or 35, wherein the GS has reduced activity compared to a wild-type Alligator GS.

Embodiment 37: The vector of embodiment 33, wherein the GS has the amino acid sequence of SEQ ID NO:1.

Embodiment 38: The vector of any one of embodiments 34 to 37, wherein the GS-encoding sequence is at least 80% identical to SEQ ID NO:5.

Embodiment 39: The vector of embodiment 33, wherein the GS is a green anole GS derived from Dactyloidae.

Embodiment 40: The vector of embodiment 39, wherein the GS has an amino acid sequence that is at least 90%, at least 92%, at least 95%, at least 98%, at least 99%, or 100% identical to SEQ ID NO:2.

Embodiment 41: The vector of embodiment 39 or 40, wherein the GS has reduced activity compared to a wild-type green anole GS.

Embodiment 42: The vector of embodiment 33, wherein the GS has the amino acid sequence of SEQ ID NO:2.

Embodiment 43: The vector of any one of embodiments 39 to 42, wherein the GS-encoding sequence is at least 80% identical to SEQ ID NO:6.

Embodiment 44: The vector of embodiment 33, wherein the GS is a spotted gar GS derived from Lepisosteidae.

Embodiment 45: The vector of embodiment 44, wherein the GS has an amino acid sequence that is at least 90%, at least 92%, at least 95%, at least 98%, at least 99%, or 100% identical to SEQ ID NO:3.

Embodiment 46: The vector of embodiment 44 or 45, wherein the GS has reduced activity compared to a wild-type spotted gar GS.

Embodiment 47: The vector of embodiment 33, wherein the GS has the amino acid sequence of SEQ ID NO:3.

Embodiment 48: The vector of any one of embodiments 44 to 47, wherein the GS-encoding sequence is at least 80% identical to SEQ ID NO:7.

Embodiment 49: The vector of embodiment 33, wherein the amino acid sequence of the GS comprises SEQ ID NO:4 with an amino acid substitution at a position selected from the group consisting of: H8, N10, G12, I13, Q15, M16, S19, E24, V33, G39, C49, C53, V54, E56, F68, S72, S80, V82, F85, E92, F98, F102, Q106, K107, P108, L113, H115, T116, K118, S125, Q127, H128, L139, D152, L160, R172, M176, K189, T191, Y194, K198, H199, I206, C209, R213, V220, K230, 1235, A236, T237, S240, T260, N265, H269, K271, A273, K276, S278, K279, R282, A287, F303, H304, K305, N308, N310, D311, D318, S320, T328, E332, A339, C341, F349, A350, I355, V356, N362, T364, Q367, F369, and Q370.

Embodiment 50: The vector of embodiment 33, wherein the amino acid sequence of the GS comprises SEQ ID NO:4 with an amino acid substitution at a position selected from the group consisting of: N10, G12, S19, V33, C49, C53, V54, E56, S72, S80, V82, E92, F98, Q106, H128, D152, L160, R172, M176, T191, Y194, K198, H199, I206, R213, V220, K230, T237, S240, T260, K271, K305, D311, D318, S320, T328, A339, C341, F349, I355, Q367, and Q370.

Embodiment 51: The vector of embodiment 50, wherein the amino acid sequence of the GS comprises SEQ ID NO:4 with about 3, about 5, about 10, about 15, about 20, about 25, about 30, about 35, or about 40 amino acid substitutions at positions selected from the group consisting of: N10, G12, S19, V33, C49, C53, V54, E56, S72, S80, V82, E92, F98, Q106, H128, D152, L160, R172, M176, T191, Y194, K198, H199, I206, R213, V220, K230, T237, S240, T260, K271, K305, D311, D318, S320, T328, A339, C341, F349, I355, Q367, and Q370.

Embodiment 52: A DNA vector suitable for recombinant protein production or genomic integration, comprising a nucleotide sequence encoding a GS (a GS-encoding sequence), wherein the GS comprises a catalytic domain from an Alligator GS, a green anole GS, or a spotted gar GS.

Embodiment 53: The vector of embodiment 52, wherein the GS comprises a catalytic domain from an Alligator GS having an amino acid sequence that is at least 90%, at least 92%, at least 95%, at least 98%, at least 99%, or 100% identical to SEQ ID NO:1.

Embodiment 54: The vector of embodiment 53, wherein the catalytic domain has an amino acid sequence that is at least 90%, at least 92%, at least 95%, at least 98%, at least 99%, or 100% identical to amino acids 110-359 of SEQ ID NO:1.

Embodiment 55: The vector of embodiment 52, wherein the GS comprises a catalytic domain from a green anole GS having an amino acid sequence that is at least 90%, at least 92%, at least 95%, at least 98%, at least 99%, or 100% identical to SEQ ID NO:2.

Embodiment 56: The vector of embodiment 55, wherein the catalytic domain has an amino acid sequence that is at least 90%, at least 92%, at least 95%, at least 98%, at least 99%, or 100% identical to amino acids 110-359 of SEQ ID NO:2.

Embodiment 57: The vector of embodiment 52, wherein the GS comprises a catalytic domain from a spotted gar GS having an amino acid sequence that is at least 90%, at least 92%, at least 95%, at least 98%, at least 99%, or 100% identical to SEQ ID NO:3.

Embodiment 58: The vector of embodiment 57, wherein the catalytic domain has an amino acid sequence that is at least 90%, at least 92%, at least 95%, at least 98%, at least 99%, or 100% identical to amino acids 113-362 of SEQ ID NO:3.

Embodiment 59: The vector of any one of embodiments 33 to 58, wherein the GS-encoding sequence is operatively linked to an mRNA-destabilizing element.

Embodiment 60: The vector of any one of embodiments 33 to 58, wherein the GS comprises a degron.

Embodiment 61: The vector of embodiment 60, wherein the degron has an amino acid sequence selected from the group consisting of SEQ ID NOs:13-15.

Embodiment 62: The vector of any one of embodiments 33 to 61, wherein the vector is suitable for recombinant protein production and further comprises an expression cassette.

Embodiment 63: The vector of embodiment 62, wherein the GS-encoding sequence is operatively linked to Simian vacuolating virus 40 (SV40) promoter.

Embodiment 64: The vector of embodiment 62 or 63, wherein the GS-encoding sequence is operatively linked to a poly(A) tail.

Embodiment 65: The vector of any one of embodiments 62 to 64, comprising two or more expression cassettes.

Embodiment 66: The vector of any one of embodiments 62 to 65, wherein the expression cassette comprises a nucleotide sequence encoding a POI (POI-encoding sequence).

Embodiment 67: The vector of embodiment 66, wherein the POI is an antibody, an enzyme, a soluble protein, a secreted protein, a membrane protein, or a fusion protein.

Embodiment 68: The vector of embodiment 67, wherein the POI is an antibody selected from the group consisting of an IgG1 antibody, an IgG2 antibody, an IgG3 antibody, an IgG4 antibody, an IgA antibody, an IgM antibody, a Fab, a Fab′, a F(ab′)2, a Fv, a scFv, a (scFv)2, a single domain antibody (sdAb), a single chain antibody (scAb), and a heavy chain antibody (HCAb).

Embodiment 69: The vector of embodiment 67, wherein the POI is an antibody selected from the group consisting of a monoclonal antibody, a bispecific antibody, a multi-specific antibody, a bivalent antibody, and a multivalent antibody.

Embodiment 70: The vector of any one of embodiments 66 to 69, wherein the POI consists of one or more copies of the same polypeptide.

Embodiment 71: The vector of any one of embodiments 66 to 69, wherein the POI comprises two different polypeptides.

Embodiment 72: The vector of any one of embodiments 71, wherein the POI is an antibody comprising a light chain and a heavy chain, each encoded by a separate nucleotide sequence on the vector.

Embodiment 73: The vector of any one of embodiments 33 to 60, wherein the vector is suitable for genomic integration.

Embodiment 74: Use of the vector of any one of embodiments 66 to 72 for identifying host cells capable of producing the POI.

Embodiment 75: Use of the vector of embodiment 73 for identifying a genomic locus with high transcriptional activity.

Embodiment 76: A method for identifying a host cell capable of producing a POI, comprising introducing the vector of any one of embodiments 66 to 72 into a population of host cells, culturing the population of host cells in a glutamine-free medium, wherein the host cell capable of growing in the culture medium is identified as the host cell capable of producing the POI.

Embodiment 77: A method for identifying a genomic locus with high transcriptional activity, comprising introducing the vector of embodiment 73 into a population of host cells, culturing the population of host cells in a glutamine-free medium, wherein the host cell capable of growing in the culture medium is identified as the host cell having the GS-encoding sequence inserted at a genomic locus with high transcriptional activity.

Embodiment 78: The method of embodiment 77, further comprising sequencing the genome of the identified host cell to locate the genomic locus with high transcriptional activity

Embodiment 79: The method of any one of embodiments 76 to 78, wherein the population of host cells are cultured in the presence of a GS inhibitor.

Embodiment 80: The method of embodiment 79, wherein the GS inhibitor is methionine sulfoximine (MSX).

Embodiment 81: A host cell comprising the vector of any one of embodiments 66 to 72.

Embodiment 82: The host cell of embodiment 81 having a wild-type endogenous GS.

Embodiment 83: The host cell of embodiment 81 wherein the endogenous GS of the host cell has reduced activity or is knocked out.

Embodiment 84: The host cell of any one of embodiments 81 to 83, wherein the host cell is a mammalian cell.

Embodiment 85: The host cell of embodiment 84 that is a CHO cell.

Embodiment 86: Use of the host cell of any one of embodiments 81 to 85 for in vitro production of the POI.

Embodiment 87: An in vitro method of producing a POI comprising culturing the host cell of any one of embodiments 81 to 85 under conditions and for sufficient time to produce the POI.

Embodiment 88: An in vitro method of producing a POI comprising replacing the GS-encoding sequence with a POI-encoding sequence in the host cell identified in the method of embodiment 77, and culturing the host cell under conditions and for sufficient time to produce the POI.

Embodiment 89: The method of embodiment 87 or 88, further comprising separating the POI from other components in the culture.

Embodiment 90: The method of embodiment 89, wherein the separating comprises extraction, continuous liquid-liquid extraction, pervaporation, membrane filtration, membrane separation, reverse osmosis, electrodialysis, distillation, crystallization, centrifugation, extractive filtration, ion exchange chromatography, absorption chromatography, or ultrafiltration.

Embodiment 91: An expression system for in vitro production of a POI comprising the DNA vector of any one of embodiments 33 to 73, and a host cell.

Embodiment 92: The expression system of embodiment 91, wherein the host cell is a CHO cell.

Embodiment 93: The expression system of embodiment 91 or 92, further comprising a glutamine-free culture medium.

Embodiment 94: The expression system of any one of embodiments 91 to 93, further comprising a GS inhibitor.

Embodiment 95: The expression system of any one of embodiments 91 to 94, further comprising a means for introducing the vector into the host cell.

Embodiment: The expression system of any one of embodiments 91 to 95 that is contained in a kit.

### 5.8 Experimental

The examples provided below are for purposes of illustration only, which are not intended to be limiting unless otherwise specified. Thus, the invention should in no way be construed as being limited to the following examples, but rather, should be construed to encompass any and all variations which become evident as a result of the teaching provided herein.

### 5.8.1 Example 1: Identification of GS Markers that Significantly Improved Yield and Screening Efficiency

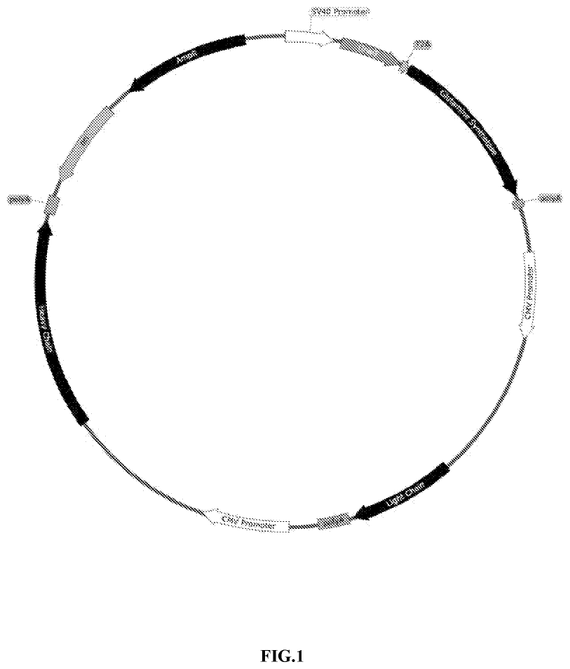

Vector design: Nearly all selectable marker currently used in GS system is Cricetulus griseus-derived GS. To investigate whether the GS from other species further improve titer or screening efficiency, a set of seven vectors was designed as described below and depicted in FIG. 1:
a sequence coding for an eel green fluorescent protein UnaG, a 2A self-cleaving peptide and a glutamine synthetase, placed under the control of the SV40 promoter;
two expression cassettes, in which the sequences coding for the heavy chain and light chain of a human anti-RANKL antibody denosumab were placed under the control of CMV promoters, respectively;
a sequence coding for a protein conferring resistance to ampicillin, for use in prokaryotic cells; and
a prokaryotic origin of replication.

The 2A self-cleaving peptide allowed relatively equal molar expression of the two products separated by it, namely, the green fluorescent protein UnaG and glutamine synthetase. After random integration, GS expression in each individual cell was evaluated by monitoring UnaG level using flow cytometry. Denosumab here served as the secreted reporter for assessing titers using selectable glutamine synthetase markers from different species. The seven vectors differed from one another by the GS-coding sequences, which were derived from Chinese hamster (Cricetulus griseus), spotted gar (Lepisosteus oculatus), Alligator (Alligator mississippiensis), and green anole (Anolis agassizii), respectively.

| | | Protein | |
|---|---|---|---|
| Vector | GS species | sequence | Nucleotide sequence |
| pZG_CLD 16 | Chinese hamster | SEQ ID NO: 4 | SEQ ID NO: 8 |
| | (Cricetulus | Uniprot# | Derived from NCBI# |
| | griseus) | P04773 | NM_001246770 |
| pZG_CLD 33 | Alligator | SEQ ID NO: 1 | SEQ ID NO: 5 |
| | (Alligator | Uniprot# | Derived from NCBI# |
| | mississippiensis) | A0A151MI87 | XM_007480917 |
| pZG_CLD_34 | Green anole | SEQ ID NO: 2 | SEQ ID NO: 6 |
| | (Anolis | Uniprot# | Derived from NCBI# |
| | carolinensis) | H9GDW2 | XM_003228747.2 |
| pZG_CLD_36 | Spotted gar | SEQ ID NO: 3 | SEQ ID NO: 7 |
| | (Lepisosteus | Uniprot# | Derived from NCBI# |
| | oculatus) | W5MAD8 | NM_006634692.2 |

Electroporation and cell screening: Expression vectors described above were first linearized by the restriction endonuclease PvuI (there was one PvuI site in the ampicillin resistant gene). Then linearized vectors were introduced into the host cell using cell electroporator (Lonza Nuleofector 2b). The host cell was derived from ATCC wild-type Chinese Hamster Ovary CHO-K1 (CCL-61) and adapted to serum-free suspension culture at Shanghai ZhenGe Biotech Co. Ltd. 24 hours post electroporation, cell pools were plated into culture flasks or 96-well plates (approximately 13,000 cells/well, 300 wells per condition) in the absence of or presence of MSX (50 μM).

### 5.8.2 Example 2: Analysis of Recombinant Protein Expression

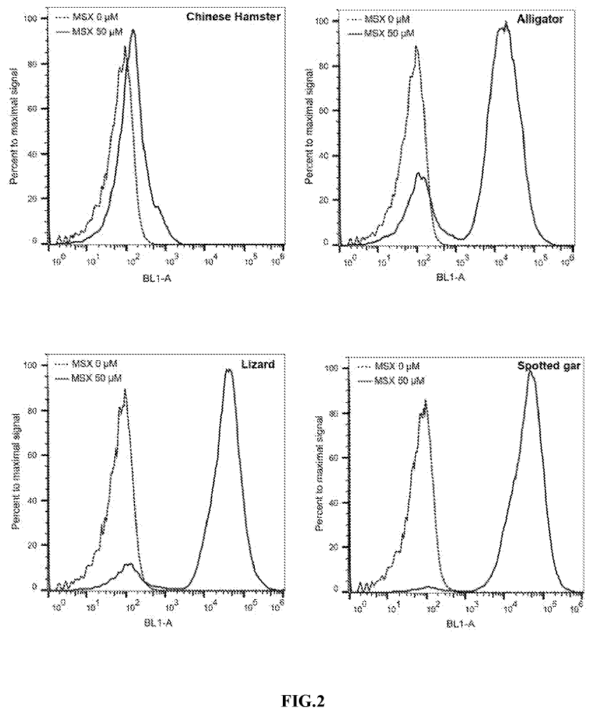

Flow cytometry analysis of GS expression: About 3 weeks post-seeding, cell pools electroporated with GS gene from indicated species were analyzed using Attune N×T flow cytometer (FIG. 2). The 2A self-cleaving peptides between the green fluorescent protein UnaG and GS resulted in the equal molar expression of both proteins, allowing the direct comparison of GS expression by monitoring UnaG fluorescent signals. As shown, when the GS from Chinese Hamster was used as the selectable marker, most tested cells (>98%) remained UnaG “negative” in the presence of the MSX, indicating low selection efficiency. Surprisingly, when using the GS from spotted gar, Alligator, or green anole as the selectable marker, significant amounts of cells showed positive UnaG expression, demonstrating that GS markers from these species greatly improved MSX selection efficiencies compared with the traditional Chinese hamster GS (FIG. 2). Furthermore, UnaG signal intensities were 100 folds or higher stronger when GS markers from these species were used as the selectable marker, indicating that the GS expression (proportional to the expression of recombinant POI) were also at least 100 folds higher.

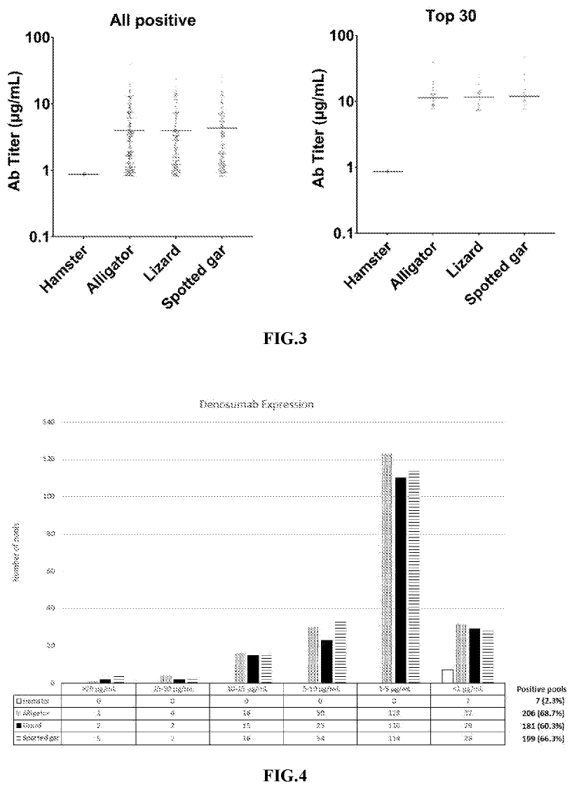

Expression analysis: Twenty-one days post-seeding, supernatants from cell pools were diluted 5 folds then analyzed using Octet Qke Label-free system and Octet ProA biosensor (Sartorius, Cat No. 18-5010) according to the manufacturer's instruction. As shown in FIG. 3, denosumab expression from all positive pools (with >0.125 μg/mL denosumab expression) or the top 30 pools using indicated GS were plotted. When the traditional Chinese hamster GS was used as the selectable marker, at Day 21, among all 300 seeded pools, only 2.3% pools (7) expressed detectable denosumab (>0.125 μg/mL), the median expression value being 0.86 μg/mL. By contrast, when the GS from Alligator, lizard (green anole) and spotted gar were used as the selectable marker, 68.7% (206), 60.3% (181) and 66.3% (199) pools expressed denosumab (>0.125 μg/mL), respectively, with the median expression values being 2.13 μg/mL, 2.14 μg/mL, and 2.14 μg/mL, respectively. Consistent with UnaG analysis above, Octet-based analysis of recombinant protein expression demonstrated that using Alligator, lizard (green anole) or spotted gar GS as the selectable marker significantly improved both screening efficiency and yield of the recombinant protein.

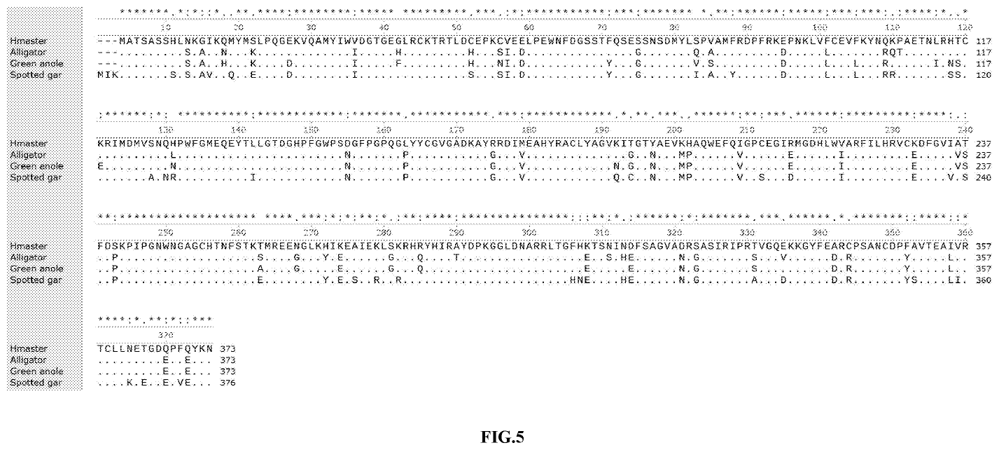

Numbers of positive pools with indicated ranges of denosumab expression were listed in FIG. 4. When using Chinese hamster GS as the selectable marker, denosumab expression from 2.3% of pools were detectable but all <1 μg/mL. when using the GS from Alligator, lizard (green anole) and spotted gar as the selectable marker, most of pools expressed denosumab >1 μg/mL and more than 5% of pools expressed more than 10 μg/mL. Thus, higher titer candidates could be acquired by screening with a smaller number of starting pools using these newly identified selectable markers.

### 6. REFERENCE TO SEQUENCE LISTING SUBMITTED ELECTRONICALLY

This application incorporates by reference a Sequence Listing with this application as an ASCII text file entitled “817A002WO02_SL.XML” created on Dec. 12, 2022 and having a size of 22,601 bytes.

What is claimed is:

1. Use of a nucleotide sequence encoding a glutamine synthetase (GS) as a selectable marker, wherein the GS is an Alligator GS, a green anole GS, or a spotted gar GS.

2. The use of claim 1, wherein the GS is an Alligator GS derived from Alligatoridae.

3. The use of claim 2, wherein the GS has an amino acid sequence that is at least 90%, at least 92%, at least 95%, at least 98%, at least 99%, or 100% identical to SEQ ID NO:1.

4. The use of claim 2 or 3, wherein the GS has reduced activity compared to a wild-type Alligator GS.

5. The use of claim 1, wherein the GS has the amino acid sequence of SEQ ID NO:1.

6. The use of claim 1, wherein the GS is a green anole GS derived from Dactyloidae.

7. The use of claim 6, wherein the GS has an amino acid sequence that is at least 90%, at least 92%, at least 95%, at least 98%, at least 99%, or 100% identical to SEQ ID NO:2.

8. The use of claim 6 or 7, wherein the GS has reduced activity compared to a wild-type green anole GS.

9. The use of claim 1, wherein the GS has the amino acid sequence of SEQ ID NO:2.

10. The use of claim 1, wherein the GS is a spotted gar GS derived from Lepisosteidae.

11. The use of claim 10, wherein the GS has an amino acid sequence that is at least 90%, at least 92%, at least 95%, at least 98%, at least 99%, or 100% identical to SEQ ID NO:3.

12. The use of claim 10 or 11, wherein the GS has reduced activity compared to a wild-type spotted gar GS.

13. The use of claim 1, wherein the GS has the amino acid sequence of SEQ ID NO:3.

14. The use of claim 1, wherein the amino acid sequence of the GS comprises SEQ ID NO:4 with an amino acid substitution at a position selected from the group consisting of: H8, N10, G12, I13, Q15, M16, S19, E24, V33, G39, C49, C53, V54, E56, F68, S72, S80, V82, F85, E92, F98, F102, Q106, K107, P108, L113, H115, T116, K118, S125, Q127, H128, L139, D152, L160, R172, M176, K189, T191, Y194, K198, H199, I206, C209, R213, V220, K230, I235, A236, T237, S240, T260, N265, H269, K271, A273, K276, S278, K279, R282, A287, F303, H304, K305, N308, N310, D311, D318, S320, T328, E332, A339, C341, F349, A350, I355, V356, N362, T364, Q367, F369, and Q370.

15. The use of claim 1, wherein the amino acid sequence of the GS comprises SEQ ID NO:4 with an amino acid substitution at a position selected from the group consisting of: N10, G12, S19, V33, C49, C53, V54, E56, S72, S80, V82, E92, F98, Q106, H128, D152, L160, R172, M176, T191, Y194, K198, H199, I206, R213, V220, K230, T237, S240, T260, K271, K305, D311, D318, S320, T328, A339, C341, F349, I355, Q367, and Q370.

16. The use of claim 15, wherein the amino acid sequence of the GS comprises SEQ ID NO:4 with about 3, about 5, about 10, about 15, about 20, about 25, about 30, about 35, or about 40 amino acid substitutions at positions selected from the group consisting of: N10, G12, S19, V33, C49, C53, V54, E56, S72, S80, V82, E92, F98, Q106, H128, D152, L160, R172, M176, T191, Y194, K198, H199, I206, R213, V220, K230, T237, S240, T260, K271, K305, D311, D318, S320, T328, A339, C341, F349, I355, Q367, and Q370.

17. Use of a nucleotide sequence encoding a GS as a selectable marker, wherein the GS comprises a catalytic domain from an Alligator GS, a green anole GS, or a spotted gar GS.

18. The use of claim 17, wherein the GS comprises a catalytic domain from an Alligator GS having an amino acid sequence that is at least 90%, at least 92%, at least 95%, at least 98%, at least 99%, or 100% identical to SEQ ID NO:1.

19. The use of claim 18, wherein the catalytic domain has an amino acid sequence that is at least 90%, at least 92%, at least 95%, at least 98%, at least 99%, or 100% identical to amino acids 110-359 of SEQ ID NO:1.

20. The use of claim 17, wherein the GS comprises a catalytic domain from a green anole GS having an amino acid sequence that is at least 90%, at least 92%, at least 95%, at least 98%, at least 99%, or 100% identical to SEQ ID NO:2.

21. The use of claim 20, wherein the catalytic domain has an amino acid sequence that is at least 90%, at least 92%, at least 95%, at least 98%, at least 99%, or 100% identical to amino acids 110-359 of SEQ ID NO:2.

22. The use of claim 17, wherein the GS comprises a catalytic domain from a spotted gar GS having an amino acid sequence that is at least 90%, at least 92%, at least 95%, at least 98%, at least 99%, or 100% identical to SEQ ID NO:3.

23. The use of claim 22, wherein the catalytic domain has an amino acid sequence that is at least 90%, at least 92%, at least 95%, at least 98%, at least 99%, or 100% identical to amino acids 113-362 of SEQ ID NO:3.

24. The use of any one of claims 1 to 23, wherein the GS-encoding nucleotide sequence is operatively linked to a mRNA destabilizing element.

25. The use of any one of claims 1 to 23, wherein the GS comprises a degron.

26. The use of claim 25, wherein the degron has an amino acid sequence selected from the group consisting of SEQ ID NOs:13-15.

27. The use of any one of claims 1 to 26, that is for identifying a genomic locus with high transcriptional activity.

28. The use of any one of claims 1 to 26, that is for identifying a host cell capable of producing a protein of interest (POI).

29. The use of any one of claims 1 to 26, that is for recombinant production of a POI.

30. The use of claim 29, that is for recombinant protein production in a mammalian cell.

31. The use of claim 30, wherein the mammalian cell is a Chinese Hamster Ovary (CHO) cell.

32. The use of any one of claims 28 to 31, wherein the POI is selected from the group consisting of an antibody, an enzyme, a soluble protein, a secreted protein, a membrane protein, and a fusion protein.

33. A deoxyribonucleic acid (DNA) vector suitable for recombinant protein production or genomic integration, comprising a nucleotide sequence encoding a GS (a GS-encoding sequence), wherein the GS is an Alligator GS, a green anole GS, or a spotted gar GS.

34. The vector of claim 33, wherein the GS is an Alligator GS derived from Alligatoridae.

35. The vector of claim 34, wherein the GS has an amino acid sequence that is at least 90%, at least 92%, at least 95%, at least 98%, at least 99%, or 100% identical to SEQ ID NO:1.

36. The vector of claim 34 or 35, wherein the GS has reduced activity compared to a wild-type Alligator GS.

37. The vector of claim 33, wherein the GS has the amino acid sequence of SEQ ID NO: 1.

38. The vector of any one of claims 34 to 37, wherein the GS-encoding sequence is at least 80% identical to SEQ ID NO:5.

39. The vector of claim 33, wherein the GS is a green anole GS derived from Dactyloidae.

40. The vector of claim 39, wherein the GS has an amino acid sequence that is at least 90%, at least 92%, at least 95%, at least 98%, at least 99%, or 100% identical to SEQ ID NO:2.

41. The vector of claim 39 or 40, wherein the GS has reduced activity compared to a wild-type green anole GS.

42. The vector of claim 33, wherein the GS has the amino acid sequence of SEQ ID NO:2.

43. The vector of any one of claims 39 to 42, wherein the GS-encoding sequence is at least 80% identical to SEQ ID NO:6.

44. The vector of claim 33, wherein the GS is a spotted gar GS derived from Lepisosteidae.

45. The vector of claim 44, wherein the GS has an amino acid sequence that is at least 90%, at least 92%, at least 95%, at least 98%, at least 99%, or 100% identical to SEQ ID NO:3.

46. The vector of claim 44 or 45, wherein the GS has reduced activity compared to a wild-type spotted gar GS.

47. The vector of claim 33, wherein the GS has the amino acid sequence of SEQ ID NO:3.

48. The vector of any one of claims 44 to 47, wherein the GS-encoding sequence is at least 80% identical to SEQ ID NO:7.

49. The vector of claim 33, wherein the amino acid sequence of the GS comprises SEQ ID NO:4 with an amino acid substitution at a position selected from the group consisting of: H8, N10, G12, I13, Q15, M16, S19, E24, V33, G39, C49, C53, V54, E56, F68, S72, S80, V82, F85, E92, F98, F102, Q106, K107, P108, L113, H115, T116, K118, S125, Q127, H128, L139, D152, L160, R172, M176, K189, T191, Y194, K198, H199, I206, C209, R213, V220, K230, I235, A236, T237, S240, T260, N265, H269, K271, A273, K276, S278, K279, R282, A287, F303, H304, K305, N308, N310, D311, D318, S320, T328, E332, A339, C341, F349, A350, I355, V356, N362, T364, Q367, F369, and Q370.

50. The vector of claim 33, wherein the amino acid sequence of the GS comprises SEQ ID NO:4 with an amino acid substitution at a position selected from the group consisting of: N10, G12, S19, V33, C49, C53, V54, E56, S72, S80, V82, E92, F98, Q106, H128, D152, L160, R172, M176, T191, Y194, K198, H199, I206, R213, V220, K230, T237, S240, T260, K271, K305, D311, D318, S320, T328, A339, C341, F349, I355, Q367, and Q370.

51. The vector of claim 50, wherein the amino acid sequence of the GS comprises SEQ ID NO:4 with about 3, about 5, about 10, about 15, about 20, about 25, about 30, about 35, or about 40 amino acid substitutions at positions selected from the group consisting of: N10, G12, S19, V33, C49, C53, V54, E56, S72, S80, V82, E92, F98, Q106, H128, D152, L160, R172, M176, T191, Y194, K198, H199, I206, R213, V220, K230, T237, S240, T260, K271, K305, D311, D318, S320, T328, A339, C341, F349, I355, Q367, and Q370.

52. A DNA vector suitable for recombinant protein production or genomic integration, comprising a nucleotide sequence encoding a GS (a GS-encoding sequence), wherein the GS comprises a catalytic domain from an Alligator GS, a green anole GS, or a spotted gar GS.

53. The vector of claim 52, wherein the GS comprises a catalytic domain from an Alligator GS having an amino acid sequence that is at least 90%, at least 92%, at least 95%, at least 98%, at least 99%, or 100% identical to SEQ ID NO:1.

54. The vector of claim 53, wherein the catalytic domain has an amino acid sequence that is at least 90%, at least 92%, at least 95%, at least 98%, at least 99%, or 100% identical to amino acids 110-359 of SEQ ID NO:1.

55. The vector of claim 52, wherein the GS comprises a catalytic domain from a green anole GS having an amino acid sequence that is at least 90%, at least 92%, at least 95%, at least 98%, at least 99%, or 100% identical to SEQ ID NO:2.

56. The vector of claim 55, wherein the catalytic domain has an amino acid sequence that is at least 90%, at least 92%, at least 95%, at least 98%, at least 99%, or 100% identical to amino acids 110-359 of SEQ ID NO:2.

57. The vector of claim 52, wherein the GS comprises a catalytic domain from a spotted gar GS having an amino acid sequence that is at least 90%, at least 92%, at least 95%, at least 98%, at least 99%, or 100% identical to SEQ ID NO:3.

58. The vector of claim 57, wherein the catalytic domain has an amino acid sequence that is at least 90%, at least 92%, at least 95%, at least 98%, at least 99%, or 100% identical to amino acids 113-362 of SEQ ID NO:3.

59. The vector of any one of claims 33 to 58, wherein the GS-encoding sequence is operatively linked to an mRNA-destabilizing element.

60. The vector of any one of claims 33 to 58, wherein the GS comprises a degron.

61. The vector of claim 60, wherein the degron has an amino acid sequence selected from the group consisting of SEQ ID NOs:13-15.

62. The vector of any one of claims 33 to 61, wherein the vector is suitable for recombinant protein production and further comprises an expression cassette.

63. The vector of claim 62, wherein the GS-encoding sequence is operatively linked to Simian vacuolating virus 40 (SV40) promoter.

64. The vector of claim 62 or 63, wherein the GS-encoding sequence is operatively linked to a poly(A) tail.

65. The vector of any one of claims 62 to 64, comprising two or more expression cassettes.

66. The vector of any one of claims 62 to 65, wherein the expression cassette comprises a nucleotide sequence encoding a POI (POI-encoding sequence).

67. The vector of claim 66, wherein the POI is an antibody, an enzyme, a soluble protein, a secreted protein, a membrane protein, or a fusion protein.

68. The vector of claim 67, wherein the POI is an antibody selected from the group consisting of an IgG1 antibody, an IgG2 antibody, an IgG3 antibody, an IgG4 antibody, an IgA antibody, an IgM antibody, a Fab, a Fab′, a F(ab′)2, a Fv, a scFv, a (scFv)2, a single domain antibody (sdAb), a single chain antibody (scAb), and a heavy chain antibody (HCAb).

69. The vector of claim 67, wherein the POI is an antibody selected from the group consisting of a monoclonal antibody, a bispecific antibody, a multi-specific antibody, a bivalent antibody, and a multivalent antibody.

70. The vector of any one of claims 66 to 69, wherein the POI consists of one or more copies of the same polypeptide.

71. The vector of any one of claims 66 to 69, wherein the POI comprises two different polypeptides.

72. The vector of any one of claim 71, wherein the POI is an antibody comprising a light chain and a heavy chain, each encoded by a separate nucleotide sequence on the vector.

73. The vector of any one of claims 33 to 60, wherein the vector is suitable for genomic integration.

74. Use of the vector of any one of claims 66 to 72 for identifying host cells capable of producing the POI.

75. Use of the vector of claim 73 for identifying a genomic locus with high transcriptional activity.

76. A method for identifying a host cell capable of producing a POI, comprising introducing the vector of any one of claims 66 to 72 into a population of host cells, culturing the population of host cells in a glutamine-free medium, wherein the host cell capable of growing in the culture medium is identified as the host cell capable of producing the POI.

77. A method for identifying a genomic locus with high transcriptional activity, comprising introducing the vector of claim 73 into a population of host cells, culturing the population of host cells in a glutamine-free medium, wherein the host cell capable of growing in the culture medium is identified as the host cell having the GS-encoding sequence inserted at a genomic locus with high transcriptional activity.

78. The method of claim 77, further comprising sequencing the genome of the identified host cell to locate the genomic locus with high transcriptional activity

79. The method of any one of claims 76 to 78, wherein the population of host cells are cultured in the presence of a GS inhibitor.

80. The method of claim 79, wherein the GS inhibitor is methionine sulfoximine (MSX).

81. A host cell comprising the vector of any one of claims 66 to 72.

82. The host cell of claim 81 having a wild-type endogenous GS.

83. The host cell of claim 81 wherein the endogenous GS of the host cell has reduced activity or is knocked out.

84. The host cell of any one of claims 81 to 83, wherein the host cell is a mammalian cell.

85. The host cell of claim 84 that is a CHO cell.

86. Use of the host cell of any one of claims 81 to 85 for in vitro production of the POI.

87. An in vitro method of producing a POI comprising culturing the host cell of any one of claims 81 to 85 under conditions and for sufficient time to produce the POI.

88. An in vitro method of producing a POI comprising replacing the GS-encoding sequence with a POI-encoding sequence in the host cell identified in the method of claim 77, and culturing the host cell under conditions and for sufficient time to produce the POI.

89. The method of claim 87 or 88, further comprising separating the POI from other components in the culture.

90. The method of claim 89, wherein the separating comprises extraction, continuous liquid-liquid extraction, pervaporation, membrane filtration, membrane separation, reverse osmosis, electrodialysis, distillation, crystallization, centrifugation, extractive filtration, ion exchange chromatography, absorption chromatography, or ultrafiltration.

91. An expression system for in vitro production of a POI comprising the DNA vector of any one of claims 33 to 73, and a host cell.

92. The expression system of claim 91, wherein the host cell is a CHO cell.

93. The expression system of claim 91 or 92, further comprising a glutamine-free culture medium.

94. The expression system of any one of claims 91 to 93, further comprising a GS inhibitor.

95. The expression system of any one of claims 91 to 94, further comprising a means for introducing the vector into the host cell.

96. The expression system of any one of claims 91 to 95 that is contained in a kit.
